(19) United States
(12) Patent Application Publication
(10) Pub. No.: US 2007/0054887 A1
(43) Pub. Date: Mar. 8, 2007

(54) Methods for treating bladder dysfunction

(75) Inventor: Enrico Colli, Milan (IT)

Correspondence Address: EDWARDS & ANGELL, LLP, P.O. BOX 55874, BOSTON, MA, 02205, US

(73) Assignee: BioXell S.p.A, Milan (IT)

(21) Appl. No.: 10/573,164

(22) PCT Filed: Sep. 24, 2004
(86) PCT No.: PCT/US04/31532

§ 371 Date: Nov. 9, 2006

(30) Foreign Application Priority Data
Sep. 24, 2003 (GB) 0322395.5
Nov. 3, 2003 (GB) 0325598.1
Mar. 1, 2004 (GB) 0404567.0
Mar. 1, 2004 (GB) 0404571.2
Jul. 29, 2004 (GB) 0416876.1

(51) Int. Cl. A61K 31/59

(52) U.S. Cl. 514/167

(57) ABSTRACT

There is provided according to the invention the use of Vitamin D compounds such as 1-alpha-fluoro-25-hydroxy-16,23e-diene-26,27-bishomo-20-epi-cholecalciferol in the prevention or treatment of bladder dysfunction.

### RELATED APPLICATIONS

This application claims priority to the following patent applications: GB0322395.5, filed 24 Sep. 2003; GB 0325598.1, filed 3 Nov. 2003; GB0404567.0, filed 1 Mar. 2004; GB 0404571.2, filed 1 Mar. 2004; and GB 0416876.1 filed 29 Jul. 2004. Each of the aforementioned patent applications is incorporated herein in its entirety by this reference.

### BACKGROUND OF THE INVENTION

Morphological bladder changes, including a progressive de-nervation and hypertrophy of the bladder wall are frequent histological findings in patients with different bladder disorders leading to overactive bladder such as bladder disorders associated with, for example, clinical benign prostatic hyperplasia (BPH) and spinal cord injury.

The increase in tension and/or strain on the bladder observed in these conditions has been shown to be associated with cellular and molecular alterations, e.g., in cytoskeletal and contractile proteins, in mitochondrial function, and in various enzyme activities of the smooth muscle cells. The hypertrophy of the bladder wall also involves alterations in its extracellular matrix and non-smooth muscle components.

These changes in the bladder are associated with the storage (irritative) symptoms, in particular frequency, urgency, urge incontinence and nocturia. These symptoms affect the social, psychological, domestic, occupational, physical and sexual lives of the patients leading to a profound negative impact on their quality of life.

At the present time, an ideal treatment of these symptoms has not been found. Each of the therapeutic options available (for example, anti-muscarinics or alpha-blockers) is associated with disadvantages relating to their mechanism of action, which is based only on the management of symptoms and not on the treatment of the etiology of the condition. In fact, the clinical utility of some of the available agents has been limited by poor efficacy and lack of universal patient acceptance due to a number of significant side effects.

As a consequence there is a need for new treatments that provide improved clinical effectiveness by targeting the underlying etiological factor, the abnormal growth and consequent dysfunction of bladder smooth muscle cells.

As described herein, it has now surprisingly been found that vitamin D analogues can treat and prevent bladder dysfunction in disorders associated with bladder hypertrophy, such as bladder overactivity and clinical BPH. Overactive bladder, also known as detrusor overactivity or detrusor instability, involves involuntary bladder spasms. A hyperactive detrusor muscle can cause overactive bladder. Although the underlying cause of overactive bladder can be neurological disease (e.g., multiple sclerosis, Parkinson's disease, stroke, spinal cord lesions), nerve damage caused by abdominal trauma, pelvic trauma, or surgery, stroke, multiple sclerosis, infection, bladder cancer, drug side effects or enlarged prostate (BPH), in many cases the cause is idiopathic, i.e. of unknown cause.

In addition, such vitamin D related compounds have an application in the treatment of irritative voiding symptoms associated with BPH. BPH is associated not only with enlargement of the gland leading to bladder outlet obstruction (BOO) and symptoms secondary to this, but also to morphological bladder changes, including a hypertrophy of the bladder wall and progressive de-nervation. These changes lead to increased functional demands and disruption of the coordination within the bladder smooth muscle cells.

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorous homeostasis.

Studies involving the metabolism of vitamin D₃ were initiated with the discovery and chemical characterization of the plasma metabolite, 25-hydroxyvitamin D_{3 [}25(OH)D₃] (Blunt, J. W. et al. (1968) Biochemistry 6:3317-3322) and the hormonally active form, 1-alpha,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1-alpha,25(OH)₂D₃ in a carefully regulated fashion (Fraser, D. R. and Kodicek, E. (1970) Nature 288:764-766; Wong, R. G. et al. (1972) J. Clin. Invest. 51:1287-1291), and the discovery of a nuclear receptor for 1-alpha,25(OH)₂D₃ (VDR) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y. and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1-alpha,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann. NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1-alpha,25(OH)₂D₃ to generate the requisite specific biological responses for this secosteroid hormone (Pike, J. W. (1991) Annu. Rev. Nutr. 11:189-216). To date, there is evidence that nuclear receptors for 1-alpha,25(OH)₂D₃ (VD₃R) exist in more than 30 tissues and cancer cell lines (Reichel, H. and Norman, A. W. (1989) Annu. Rev. Med. 40:71-78), including the normal bladder.

Vitamin D₃ and its hormonally active forms are well-known regulators of calcium and phosphorous homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1-alpha,25(OH)₂D₃ has been suggested by the combined presence of enzymes capable of oxidizing vitamin D₃ into its active forms, e.g., 25-(OH)D-1α-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40:71-78).

Given the activities of vitamin D₃ and its metabolites, much attention has focused on the development of synthetic analogues of these compounds. A large number of these analogues involve structural modifications in the A ring, B ring, C/D rings, and, primarily, the side chain (Bouillon, R. et al. (1995) Endocr. Rev. 16(2):200-257). Although a vast majority of the vitamin D₃ analogues developed to date involve structural modifications in the side chain, a few studies have reported the biological profile of A-ring diastereomers (Norman, A. W. et al. (1993) J. Biol. Chem. 268 (27):20022-20030). Furthermore, biological esterification of steroids has been studied (Hochberg, R. B. (1998) Endocr. Rev. 19(3): 331-348), and esters of vitamin D₃ are known (WO 97/11053).

Moreover, despite much effort in developing synthetic analogues, clinical applications of vitamin D and its structural analogues have been limited by the undesired side effects elicited by these compounds after administration to a subject for known indications/applications of vitamin D compounds.

The activated form of vitamin D, vitamin D₃, and some of its analogues have been described as potent regulators of cell growth and differentiation. It has previously been found that vitamin D₃, as well as an analogue (analogue V, referred to elsewhere herein as Compound B), inhibited BPH cell proliferation and counteracted the mitogenic activity of potent growth factors for BPH cells, such as keratinocyte growth factor (KGF) and insulin-like growth factor (IGFI). Moreover, the analogue induced bcl-2 protein expression, intracellular calcium mobilization, and apoptosis in both unstimulated and KGF-stimulated BPH cells.

U.S. Pat. No. 5,939,408 and EP808833 disclose a number of 1,25(OH)₂D₃ analogues including the compound 1-alpha-fluoro-25-hydroxy-16,23E-diene-26,27-bishomo-20-epi-cholecalciferol (Compound A). U.S. Pat. No. 5,939,408 and EP808833 disclose that the compounds induce differentiation and inhibition of proliferation in various skin and cancer cell lines and are useful for the treatment of hyperproliferative skin diseases such as psoriasis, neoplastic diseases such a leukemia, breast cancer and sebaceous gland diseases such as acne and seborrheic dermatitis and osteoporosis.

An apparent effect of calcitriol (1,25-dihydroxycholecalciferol) on transitional cell carcinoma of the bladder in vitro and in vivo was discussed in Konety, B. R. et al. (2001) J. Urology 165(1):253-258.

### BRIEF DESCRIPTION OF THE DRAWINGS

The present invention is further described below with reference to the following non-limiting examples and with reference to the following figures, in which:

FIG. 1 shows the immunohistochemical detection of vitamin D receptors (VDRs) in bladder tissue.

FIG. 2 shows the effect of calcitriol on bladder cell growth. “hB”=human bladder; “T”=testosterone; “C”=control.

FIG. 3 shows the effect of a vitamin D compound on testosterone-stimulated bladder cell growth. “hB”=human bladder.

FIG. 4 shows the effect of different compounds on stimulated and basal bladder cell growth. “T 10 nM”=testosterone; “F 1 nM”=finasteride; “Cyp 100 nM”=cyproterone acetate.

FIGS. 5-7 show the effect of Compound A on basal and stimulated hBC proliferation and apoptosis

FIGS. 8-11 show the effect of Compound A on desmin gene and protein expression in hBC

FIGS. 12-15 show the effect of Compound A on vitamin gene and protein expression in hBC

FIG. 16 show the effect of a vitamin D compound on bladder weight.

FIG. 17 shows the effect of a vitamin D compound on spontaneous non-voiding contraction frequency.

FIG. 18 shows the effect of a vitamin D compound on spontaneous non-voiding contraction amplitude.

FIG. 19 shows the effect of a vitamin D compound on micturition pressure.

FIG. 20 shows the effect of a vitamin D compound on residual urine.

FIG. 21 shows the effect of a vitamin D compound on the contractile response of bladder strips to EFS (Electrical Field Stimulation).

FIG. 22 shows a comparison between cystometric parameters recorded in rats treated with a vitamin D₃ analogue “Compound C” and control (vehicle treated) rats.

FIG. 23 shows the results of measuring bladder capacity in the in vivo model of cyclophosphamide (CYP) induced chronic IC in rats (control v Comp A).

FIG. 24 shows the results of measuring number of non-voiding bladder contractions in the in vivo model-cyclophosphamide (CYP) induced chronic IC in rats (control v Comp A).

### SUMMARY OF THE INVENTION

The Inventors have now surprisingly found, as demonstrated in the Examples herein, that calcitriol and other vitamin D analogues are effective in inhibiting the basal and stimulated growth of normal (i.e., non-tumor) human bladder cells.

Thus the invention provides vitamin D compounds, and new methods of treatment using such compounds, for the prevention or treatment of bladder dysfunction. More particularly, the invention provides the use of vitamin D compounds for the manufacture of a medicament for the prevention and/or treatment of bladder dysfunction, especially dysfunction related to morphological bladder changes.

The invention also provides a method for preventing and/or treating bladder dysfunction, especially dysfunction related to morphological bladder changes, by administering a vitamin D compound in an amount effective to prevent and/or to treat such dysfunction alone or in combination with further agents.

The invention still further provides a kit containing a Vitamin D compound together with instructions directing administration of the Vitamin D compound to a patient in need of prevention or treatment of bladder dysfunction thereby to prevent or treat bladder dysfunction in said patient.

### DETAILED DESCRIPTION OF THE INVENTION

I. Definitions

Before further description of the present invention, and in order that the invention may be more readily understood, certain terms are first defined and collected here for convenience.

By “bladder dysfunction” it is meant bladder conditions associated with overactivity of the detrusor muscle, for example, clinical BPH or overactive bladder. In the context of the present invention “bladder dysfunction” excludes bladder cancer.

Bladder dysfunction is usually characterised clinically by irritative symptoms (e.g., irritative storage symptoms, i.e. non voiding of the bladder). In current clinical practice, a diagnosis of overactive bladder is based upon the symptoms presented by the patient. Further urodynamic investigation may be used to confirm overactivity of the detrusor muscle.

According to the invention the vitamin D compound may be used to treat bladder dysfunction in males. Such males may concurrently suffer from BPH. Alternatively they may not suffer from BPH. According to the invention the vitamin D compound may also be used to treat bladder dysfunction in females (for example overactive bladder).

Those skilled in the art will recognise that the vitamin D compound may be used in human or veterinary medicine. It is preferred that the vitamin D compound be used in the treatment of human patients.

Without wishing to be bound by theory, the Inventors believe that a mechanism by which vitamin D analogues can be used to treat such diseases involves restricting abnormal (non-malignant) proliferation of stromal and muscular cells of the bladder, which can lead to bladder dysfunction. However, the Inventors cannot exclude additional mechanisms of action for the compounds of the invention such as via an effect on the peripheral nervous system.

The term “administration” or “administering” includes routes of introducing the vitamin D compound(s) to a subject to perform their intended function. Examples of routes of administration which can be used include injection (subcutaneous, intravenous, parenterally, intraperitoneally), oral, inhalation, rectal, vaginal, transdermal or via bladder instillation. The pharmaceutical preparations are, of course, given by forms suitable for each administration route. For example, the preparations may be administered orally in tablets or capsule form, by injection, inhalation, topically as a lotion or ointment, rectally as a suppository etc. Oral administration is preferred. The injection can be bolus or can be continuous infusion. Depending on the route of administration, the vitamin D compound can be coated with or disposed in a selected material to protect it from natural conditions which may detrimentally effect its ability to perform its intended function. The vitamin D compound can be administered alone, or in conjunction with either another agent as described above, for example with other bladder function active agents known in the art such as a smooth muscle relaxant (such as alpha blockers or anti-muscarinic drugs) or with a pharmaceutically-acceptable carrier, or both. The vitamin D compound can be administered prior to the administration of the other agent, simultaneously with the agent, or after the administration of the agent. Furthermore, the vitamin D compound can also be administered in a pro-form which is converted into its active metabolite, or more active metabolite in vivo.

The term “effective amount” includes an amount effective, at dosages and for periods of time necessary, to achieve the desired result, i.e. sufficient to treat bladder dysfunction. An effective amount of vitamin D compound may vary according to factors such as the disease state, age, gender and weight of the subject, and the ability of the vitamin D compound to elicit a desired response in the subject. Dosage regimens may be adjusted to provide the optimum therapeutic response. An effective amount is also one in which any toxic or detrimental effects (e.g., side effects) of the vitamin D compound are outweighed by the therapeutically beneficial effects.

A therapeutically effective amount of vitamin D compound (i.e., an effective dosage) may range from about 0.001 to 30 ug/kg body weight, preferably about 0.01 to 25 ug/kg body weight, more preferably about 0.1 to 20 ug/kg body weight, and even more preferably about 1 to 10 ug/kg, 2 to 9 ug/kg, 3 to 8 ug/kg, 4 to 7 ug/kg, or 5 to 6 ug/kg body weight. The skilled artisan will appreciate that certain factors may influence the dosage required to effectively treat a subject, including but not limited to the severity of the disease or disorder, previous treatments, the general health and/or age of the subject, and other diseases present. In addition, the dose administered will also depend on the particular vitamin D compound used, the effective amount of each compound can be determined by titration methods known in the art. Moreover, treatment of a subject with a therapeutically effective amount of a vitamin D compound can include a single treatment or, preferably, can include a series of treatments. In one example, a subject is treated with a vitamin D compound in the range of between about 0.1 to 20 ug/kg body weight, once per day for a duration of six months or longer, for example for life depending on management of the symptoms and the evolution of the condition. Also, as with other chronic treatments an “on-off” or intermittent treatment regime can be considered. It will also be appreciated that the effective dosage of a vitamin D compound used for treatment may increase or decrease over the course of a particular treatment.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorus atoms replacing one or more carbons of the hydrocarbon backbone. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer e.g., 1-6 carbon atoms, such as 14 carbon atoms. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiments, the term “lower alkyl” includes a straight chain alkyl having 4 or fewer carbon atoms in its backbone, e.g., C₁-C₄ alkyl.

The terms “alkoxyalkyl”, “polyaminoalkyl” and “thioalkoxyalkyl” refer to alkyl groups, as described above, which further include oxygen, nitrogen or sulfur atoms replacing one or more carbons of the hydrocarbon backbone.

The term “aryl” as used herein, refers to the radical of aryl groups, including 5- and 6-membered single-ring aromatic groups that may include from zero to four heteroatoms selected e.g., from O, N and S, for example, benzene, pyrrole, furan, thiophene, imidazole, triazole, tetrazole, pyrazole, pyridine, pyrazine, pyridazine and pyrimidine, and the like. Aryl groups also include polycyclic fused aromatic groups (preferably 9 or 10 membered) such as naphthyl, quinolyl, indolyl, and the like. Further examples include benzoxazole and benzothiazole. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “chiral” refers to molecules which have the property of non-superimposability of the mirror image partner, while the term “achiral” refers to molecules which are superimposable on their mirror image partner.

The term “isomers” or “stereoisomers” refers to compounds which have identical chemical constitution, but differ with regard to the arrangement of the atoms or groups in space.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

As used herein, the term “halogen” designates —F, —Cl, —Br or —I; the term “sulfhydryl” or “thiol” means —SH; the term “hydroxyl” means —OH.

The term “haloalkyl” is intended to include alkyl groups as defined above that are mono-, di- or polysubstituted by halogen, e.g., fluoroalkyl such as fluoromethyl and trifluoromethyl.

The term “hydroxyalkyl” is intended to include alkyl groups as defined above that are mono-, di- or polysubstituted by hydroxy, e.g., hydroxymethyl or 2-hydroxyethyl.

The term “heteroatom” as used herein means an atom of any element other than carbon or hydrogen. Preferred heteroatoms are nitrogen, oxygen, sulfur and phosphorus especially N, O and S.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The terms “isolated” or “substantially purified” are used interchangeably herein and refer to vitamin D compounds (e.g., vitamin D₃ compounds) in a non-naturally occurring state. The compounds can be substantially free of cellular material or culture medium when naturally produced, or chemical precursors or other chemicals when chemically synthesized. In certain preferred embodiments, the terms “isolated” or “substantially purified” also refer to preparations of a chiral compound which substantially lack one of the enantiomers; i.e., enantiomerically enriched or non-racemic preparations of a molecule. Similarly, the terms “isolated epimers” or “isolated diastereomers” refer to preparations of chiral compounds which are substantially free of other stereochemical forms. For instance, isolated or substantially purified vitamin D₃ compounds include synthetic or natural preparations of a vitamin D₃ enriched for the stereoisomers having a substituent attached to the chiral carbon at position 3 of the A-ring in an alpha-configuration, and thus substantially lacking other isomers having a beta-configuration. Unless otherwise specified, such terms refer to vitamin D₃ compositions in which the ratio of alpha to beta forms is greater than 1:1 by weight. For instance, an isolated preparation of an alpha-epimer means a preparation having greater than 50% by weight of the alpha-epimer relative to the beta-epimer more preferably at least 75% by weight, and even more preferably at least 85% by weight. Of course the enrichment can be much greater than 85%, providing “substantially epimer-enriched” preparations, i.e., preparations of a compound which have greater than 90% of the alpha-epimer relative to the beta-stereoisomer, and even more preferably greater than 95%. The term “substantially free of the beta stereoisomer” will be understood to have similar purity ranges.

As used herein, the term “vitamin D compound” includes any compound that is capable of treating or preventing bladder dysfunction. Generally, compounds which are ligands for the vitamin D-receptor (VDR ligands) and which are capable of treating or preventing bladder dysfunction are considered to be within the scope of the invention. Vitamin D compounds are preferably agonists of the vitamin D receptor. Thus, vitamin D compounds are intended to include secosteroids. Examples of specific vitamin D compounds suitable for use in the methods of the present invention are further described herein. A vitamin D compound includes vitamin D₂ compounds, vitamin D₃ compounds, isomers thereof, or derivatives/analogues thereof. Preferred vitamin D compounds are vitamin D₃ compounds which are ligands of (more preferably are agonists of) the vitamin D receptor. Preferably the vitamin D compound (e.g., the vitamin D₃ compound) is a more potent agonist of the vitamin D receptor than the native ligand (i.e. the vitamin D, e.g., vitamin D₃). Vitamin D₁ compounds, vitamin D₂ compounds and vitamin D₃ compounds include, respectively, vitamin D₁, D₂, D₃ and analogues thereof.

In certain embodiments, the vitamin D compound may be a steroid, such as a secosteroid, e.g., calciol, calcidiol or calcitriol.

The term “secosteroid” is art-recognized and includes compounds in which one of the cyclopentanoperhydro-phenanthrene rings of the steroid ring structure is broken. For example, 1-alpha,25(OH)₂D₃ and analogues thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1-alpha,25(OH)₂D₃ is illustrated herein having all carbon atoms numbered using standard steroid notation.

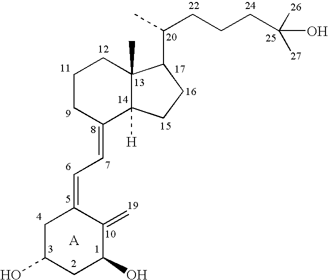

In the formulas presented herein, the various substituents on ring A are illustrated as joined to the steroid nucleus by one of these notations: a dotted line (----) indicating a substituent which is in the beta-orientation (i.e., above the plane of the ring), a wedged solid line ( [symbol] ) indicating a substituent which is in the alpha-orientation (i.e., below the plane of the molecule), or a wavy line ( [symbol] ) indicating that a substituent may be either above or below the plane of the ring. In regard to ring A, it should be understood that the stereochemical convention in the vitamin D field is opposite from the general chemical field, wherein a dotted line indicates a substituent on Ring A which is in an alpha-orientation (i.e., below the plane of the molecule), and a wedged solid line indicates a substituent on ring A which is in the beta-orientation (i.e., above the plane of the ring).

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. As shown, the A ring of the hormone 1-alpha,25(OH)₂D₃ contains two asymmetric centers at carbons 1 and 3, each one containing a hydroxyl group in well-characterized configurations, namely the 1-alpha- and 3-beta-hydroxyl groups. In other words, carbons 1 and 3 of the A ring are said to be “chiral carbons” or “chiral carbon centers.” Regardless, both configurations, cis/trans and/or Z/E are contemplated for the compounds for use in the present invention.

With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

Also, throughout the patent literature, the A ring of a vitamin D compound is often depicted in generic formulae as any one of the following structures: 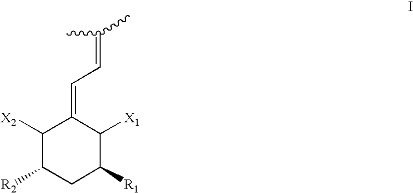
wherein X₁ and X₂ are defined as H or ═CH₂; or 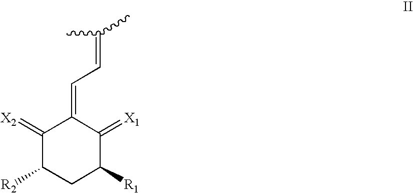
wherein X₁ and X₂ are defined as H₂ or CH₂.

Although there does not appear to be any set convention, it is clear that one of ordinary skill in the art understands either formula I or II to represent an A ring in which, for example, X₁ is ═CH₂ and X₂ is defined as H₂, as follows: 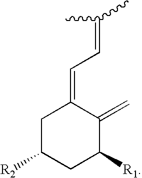
For purposes of the instant invention, formula II will be used in all generic structures.

Thus, in one aspect, the invention provides the use of a vitamin D compound in the prevention or treatment of bladder dysfunction. It provides a vitamin D compound for use in the prevention or treatment of bladder dysfunction. Also provided is a method of treating a patient with bladder dysfunction or preventing bladder dysfunction by administering an effective amount of a vitamin D compound. More particularly, there is provided a method of prevention or treatment of bladder dysfunction in a patient in need thereof by administering an effective amount of a Vitamin D compound therebv to prevent or treat bladder dysfunction in said patient. Said method typically further comprises the step of obtaining or synthesising the Vitamin D compound. The Vitamin D compound is usually formulated in a pharmaceutical composition together with a pharmaceutically acceptable diluent or carrier. Further provided is the use of a vitamin D compound in the manufacture of a medicament for the prevention or treatment of bladder dysfunction. There is also provided a kit containing a Vitamin D compound together with instructions directing administration of the Vitamin D compound to a patient in need of prevention or treatment of bladder dysfunction therebv to prevent or treat bladder dysfunction in said patient, especially wherein the Vitamin D compound is formulated in a pharmaceutical composition together with a pharmaceutically acceptable diluent or carrier.

In one embodiment, the vitamin D compound for use in accordance with the invention comprises a compound of formula I: 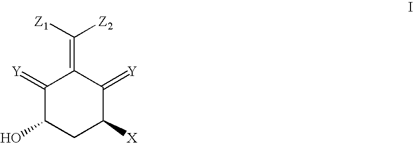
wherein
X is hydroxyl or fluoro;
Y is H₂ or CH₂;
Z₁ and Z₂ are H or a substituent represented by formula II, provided Z₁ and Z₂ are different: 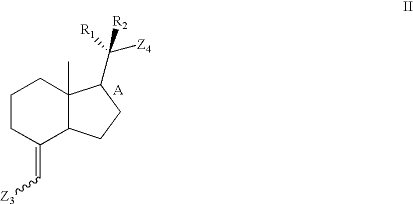
wherein
Z₃ represents the above-described formula I;
A is a single bond or a double bond;
R₁, R₂, and Z₄, are each, independently, hydrogen, alkyl, or a saturated or unsaturated carbon chain represented by formula III, provided that at least one of R₁, R₂, and Z₄ is the saturated or unsaturated carbon chain represented by formula III and provided that all of R₁, R₂, and Z₄ are not a saturated or unsaturated carbon chain represented by formula III: 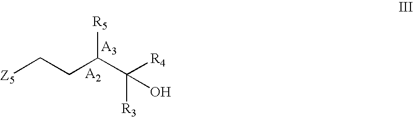
wherein
Z₅ represents the above-described formula II;
A₂ is a single bond, a double bond, or a triple bond;
A₃ is a single bond or a double bond; and
R₃, and R₄, are each, independently, hydrogen, alkyl, haloalkyl, hydroxyalkyl; and R₅ is hydrogen, H₂ or oxygen.

Thus, in the above structure (and in corresponding structures below), when A₂ represents a triple bond R₅ is absent. When A₂ represents a double bond R₅ represents hydrogen. When A₂ represents a single bond R₅ represents a carbonyl group or two hydrogen atoms.

In another embodiment, the vitamin D compound for use in accordance with the invention is a compound of formula: 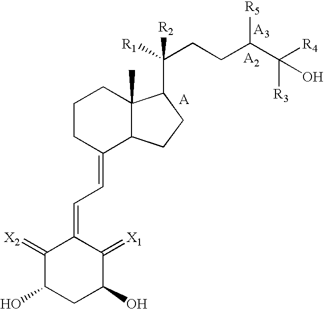
wherein:
X₁ and X₂ are H₂ or CH₂, wherein X₁ and X₂ are not CH₂ at the same time;
A is a single or double bond;
A₂ is a single, double or triple bond;
A₃ is a single or double bond;
R₁ and R₂ are hydrogen, C₁-C₄ alkyl or 4-hydroxy-4-methylpentyl, wherein R₁ and R₂ are not both hydrogen;
R₅ is hydrogen, H₂ or oxygen;
R₃ is C₁-C₄ alkyl, hydroxyalkyl or haloalkyl, e.g., fluoroalkyl, e.g., fluoromethyl or trifluoromethyl; and
R₄ is C₁-C₄ alkyl, hydroxyalkyl or haloalkyl, e.g., fluoroalkyl, e.g., fluoromethyl or trifluoromethyl.
For example, R₁ and R₂ may represent hydrogen or C₁-C₄ alkyl wherein R₁ and R₂ are not both hydrogen;

An example compound of the above structure is 1,25-dihydroxy-16-ene-23-yne cholecalciferol (elsewhere referred to herein as “Compound B”).

In yet another embodiment, the vitamin D compound for use in accordance with the invention is a “gemini” compound of the formula: 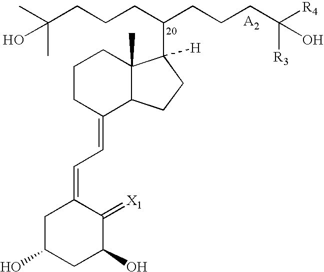
wherein:
X₁ is H₂ or CH₂;
A₂ is a single, a double or a triple bond;
R₃ is C₁-C₄ alkyl, hydroxyalkyl, or haloalkyl, e.g., fluoroalkyl, e.g., fluoromethyl or trifluoromethyl;
R₄ is C₁-C₄ alkyl, hydroxyalkyl or haloalkyl, e.g., fluoroalkyl, e.g., fluoromethyl or trifluoromethyl;
and
the configuration at C₂₀ is R or S.

An example gemini compound of the above structure is 1,25-dihydroxy-21-(3-hydroxy-3-methylbutyl)-19-nor-cholecalciferol: 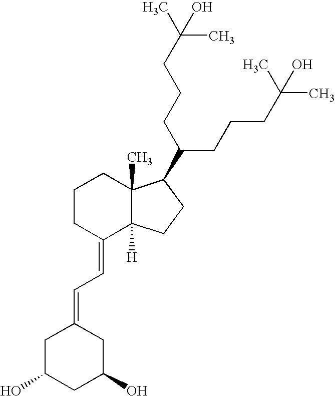
The synthesis of this compound is described in WO98/49138 which is herein incorporated in its entirety by reference.

In another embodiment, the vitamin D compound for use in accordance with the invention is a compound of the formula: 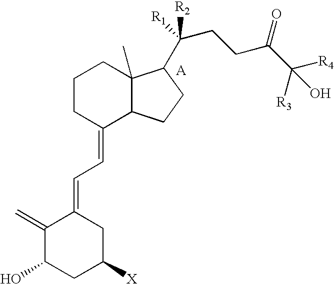
wherein:
A is a single or double bond;
R₁ and R₂ are each, independently, hydrogen or alkyl e.g., methyl;
R₃, and R₄, are each, independently, alkyl; and
X is hydroxyl or fluoro.
In a further embodiment, the vitamin D compound for use in accordance with the 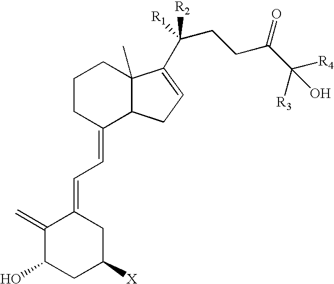
invention is a compound having the formula:
wherein:
R₁ and R₂, are each, independently, hydrogen, or alkyl, e.g., methyl;
R₃ is alkyl, e.g., methyl,
R₄ is alkyl, e.g., methyl; and
X is hydroxyl or fluoro.

In specific embodiments of the invention, the vitamin D compound for use in accordance with the invention is selected from the group consisting of:

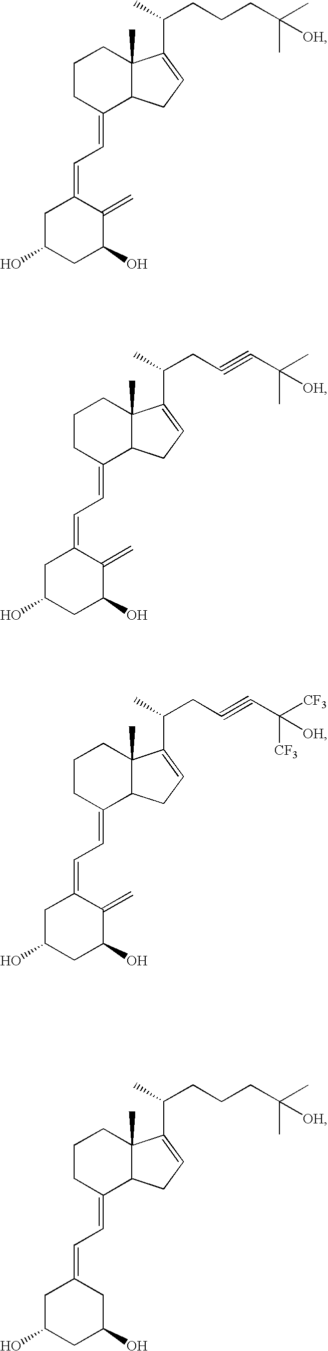

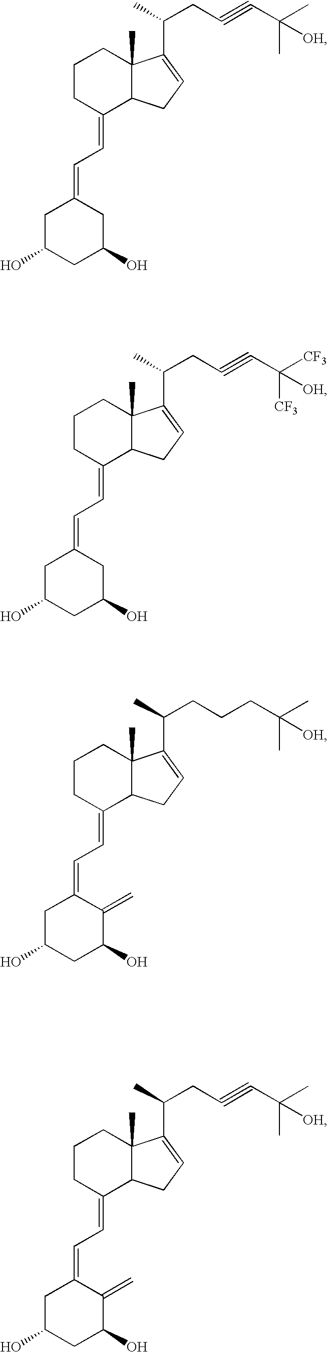

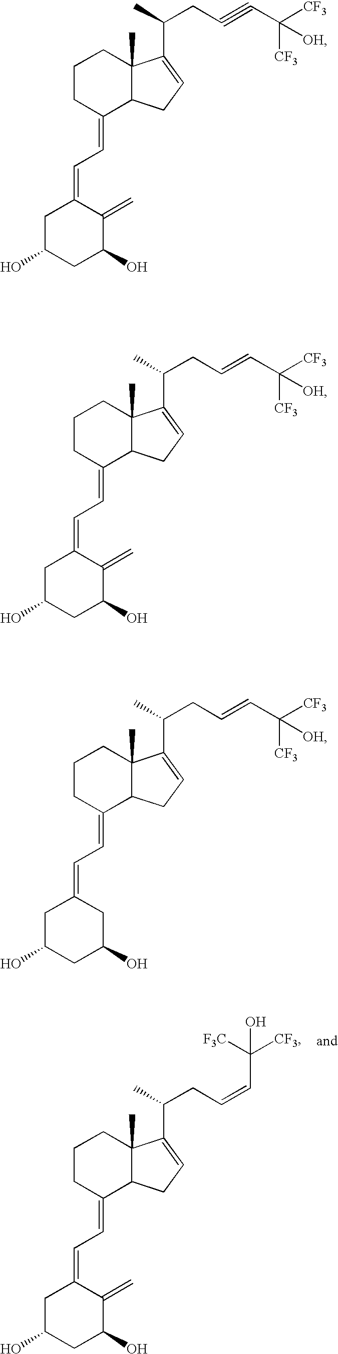

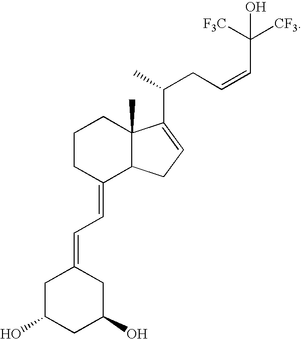

In other specific embodiments of the invention, the vitamin D compound for use in accordance with the invention is selected from the group consisting of:

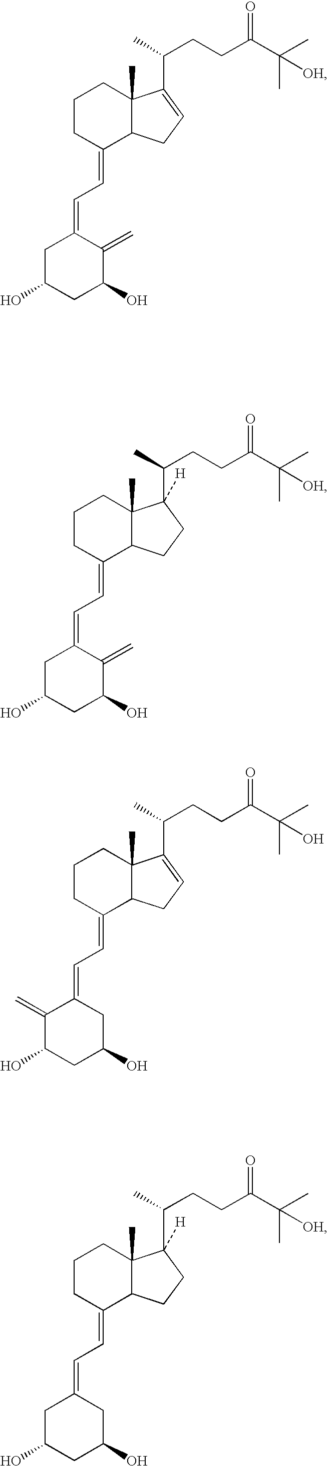

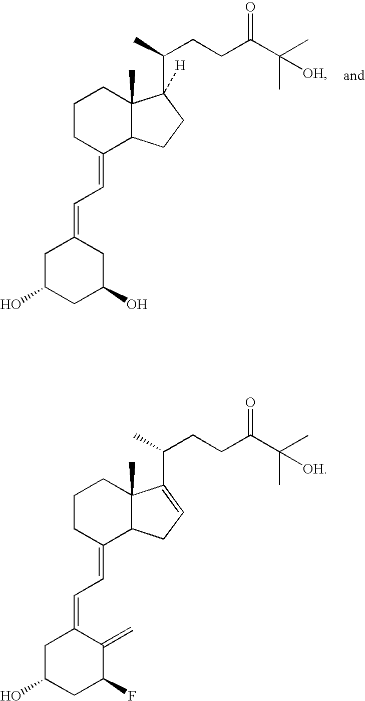

In further specific embodiments, the vitamin D compound for use in accordance with the invention is selected from the group of gemini compounds consisting of:

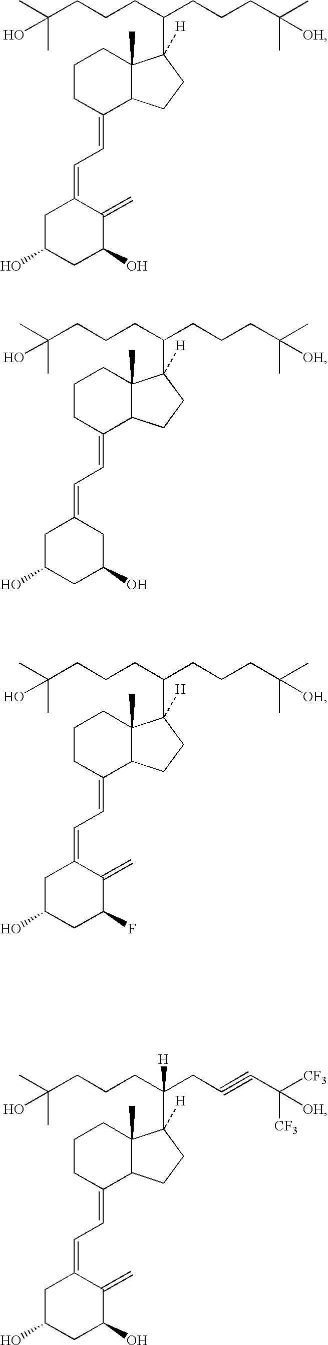

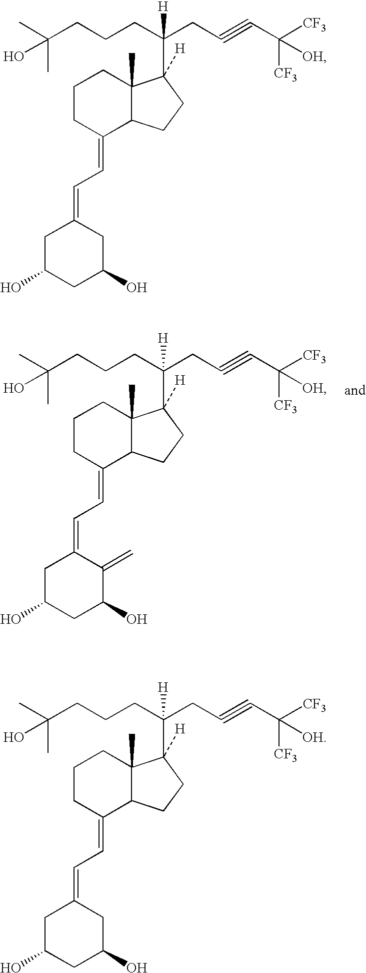

In still further specific embodiments of the invention, the vitamin D compound for use in accordance with the invention is a “Gemini” compound of the formula: 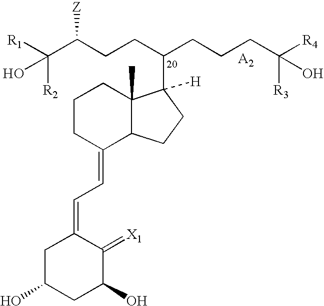
wherein:
X₁ is H₂ or CH₂;
A₂ is a single, a double or a triple bond;
R₁, R₂, R₃ and R₄ are each independently C₁-C₄ alkyl, hydroxyalkyl, or haloalkyl, e.g., fluoroalkyl, e.g., fluoromethyl or trifluoromethyl;
Z is —OH, ═O, —NH₂ or —SH;
the configuration at C₂₀ is R or S;
and pharmaceutically acceptable esters, salts, and prodrugs thereof.
Compounds of this formula may be referred to as “geminal vitamin D₃” compounds due to the presence of two alkyl chains at C20.

Z may typically represent —OH.

In a further embodiment, X₁ is CH₂. In another embodiment, A₂ is a single bond. In another, R₁, R₂, R₃, and R₄ are each independently methyl or ethyl. In a further embodiment, Z is —OH. In an example set of compounds, X₁ is CH₂; A₂ is a single bond; R₁, R₂, R₃, and R₄ are each independently methyl or ethyl; and Z is —OH. In an even further embodiment, R₁, R₂, R₃, and R₄ are each methyl.

In a further embodiment of the invention, the vitamin D compound for use in accordance with the invention is a gemini compound of the formula: 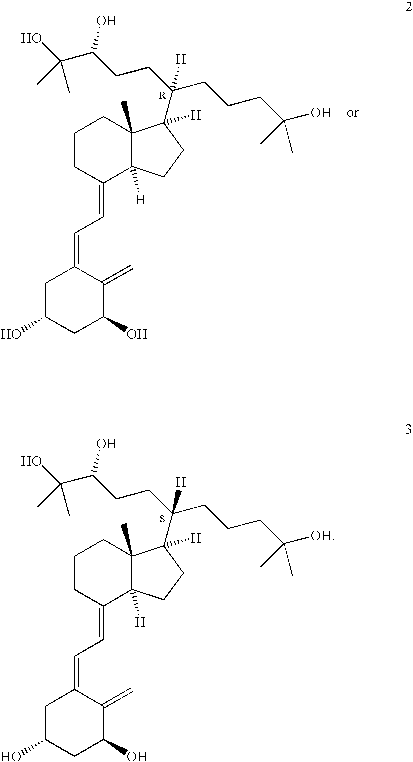
The chemical names of the compounds 2 and 3 mentioned above are:
1,25-dihydroxy-21-(2R,3-dihydroxy-3-methyl-butyl)-20R-cholecalciferol; and
1,25-dihydroxy-21-(2R,3-dihydroxy-3-methyl-butyl)-20S-cholecalciferol.

Additional embodiments of gemini compounds include the following vitamin D compounds for use in accordance with the invention.
1,25-Dihydroxy-21-(2R,3-dihydroxy-3-methyl-butyl)-20S-19-nor-cholecalciferol: 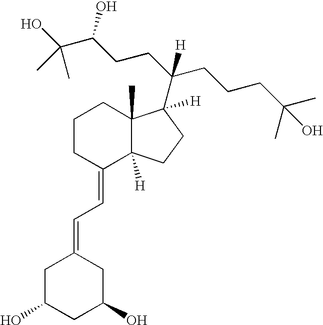
1,25-Dihydroxy-20S-21-(3-hydroxy-3-methyl-butyl)-24-keto-19-nor-cholecalciferol: 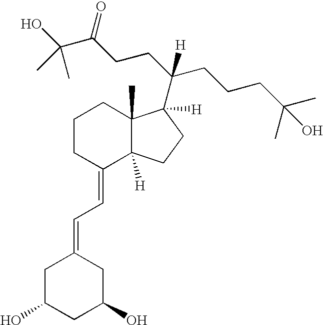
1,25-Dihydroxy-20S-21-(3-hydroxy-3-methyl-butyl)-24-keto-cholecalciferol: 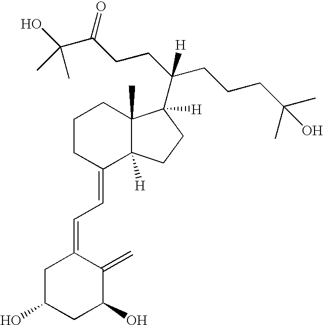
1,25-Dihydroxy-21(3-hydroxy-3-trifluoromethyl-4-trifluoro-butynyl)-26,27-hexadeutero-19-nor-20S-cholecalciferol: 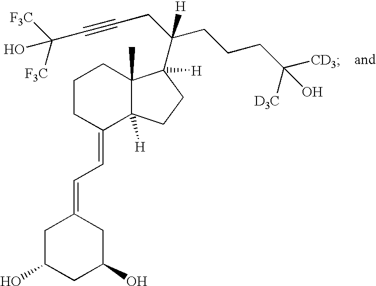
1,25-Dihydroxy-21(3-hydroxy-3-trifluoromethyl-4-trifluoro-butynyl)-26,27-hexadeutero-20S-cholecalciferol:

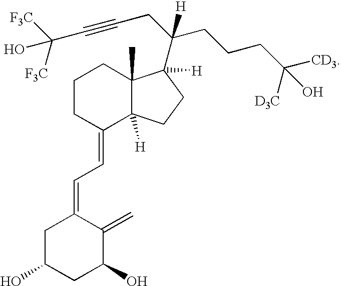

In further embodiments of the invention, the vitamin D compound for use in accordance with the invention is a compound of the formula: 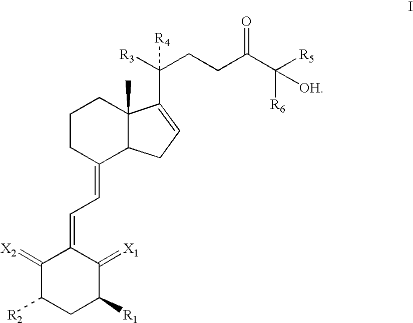
wherein:
X₁ and X₂ are each independently H₂ or CH₂, provided X₁ and X₂ are not both ═CH₂;
R₁ and R₂ are each independently hydroxyl, OC(O)C₁-C₄ alkyl, OC(O)hydroxyalkyl or OC(O)fluoroalkyl;
R₃ and R₄ are each independently hydrogen, C₁-C₄ alkyl, hydroxyalkyl or haloalkyl or
R₃ and R₄ taken together with C₂₀ form C₃-C₆ cycloalkyl; and
R₅ and R₆ are each independently C₁-C₄ alkyl, hydroxyalkyl or haloalkyl; and
pharmaceutically acceptable esters, salts, and prodrugs thereof.

R₃ and R₄ will preferably each be independently selected from hydrogen and C₁-C₄ alkyl.

In one example set of compounds R₅ and R₆ are each independently C₁-C₄ alkyl.

In another example set of compounds R₅ and R₆ are each independently haloalkyl e.g., C₁-C₄ fluoroalkyl.

When R₃ and R₄ are taken together with C₂₀ to form C₃-C₆ cycloalkyl, an example is cyclopropyl.

In one embodiment, X₁ and X₂ are each H₂. In another embodiment, R₃ is hydrogen and R₄ is C₁-C₄ alkyl. In a preferred embodiment R₄ is methyl.

In another embodiment, R₅ and R₆ are each independently methyl, ethyl, fluoromethyl or trifluoromethyl. In a preferred embodiment, R₅ and R₆ are each methyl.

In yet another embodiment, R₁ and R₂ are each independently hydroxyl or OC(O)C₁-C₄ alkyl. In a preferred embodiment, R₁ and R₂ are each OC(O)C₁-C₄ alkyl. In another preferred embodiment, R₁ and R₂ are each acetyloxy.

An example of such a compound is 1,3-O-diacetyl-1,25-dihydroxy-16-ene-24-keto-19-nor-cholecalciferol, having the following structure:

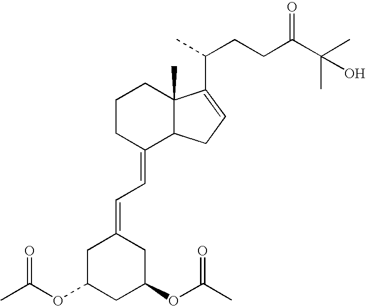

In another embodiment of the invention the vitamin D compound for use in accordance with the invention is 2-methylene-19-nor-20(S)-1-alpha-hydroxyvitamin D3: 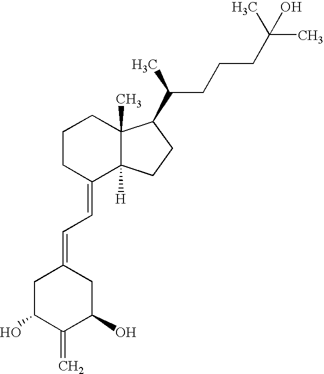
The synthesis of this compound is described in WO02/05823 and U.S. Pat. No. 5,536,713 which are herein incorporated in their entirety by reference.

In another embodiment of the invention, representing an embodiment of particular interest, the vitamin D compound for use in accordance with the invention is a compound of the formula I: 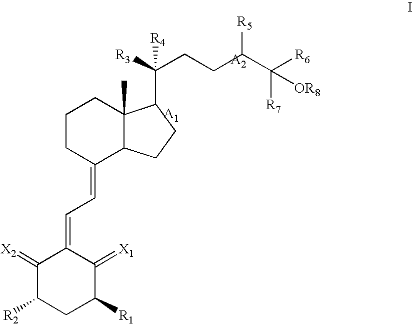
wherein:
A₁ is single or double bond;
A₂ is a single, double or triple bond;
X₁ and X₂ are each independently H₂ or CH₂, provided X₁ and X₂ are not both CH₂;
R₁ and R₂ are each independently OC(O)C₁-C₄ alkyl (including OAc), OC(O)hydroxyalkyl or OC(O)haloalkyl;
R₃, R₄ and R₅ are each independently hydrogen, C₁-C₄ alkyl, hydroxyalkyl, or haloalkyl, or R₃ and P, taken together with C₂₀ form C₃-C₆ cycloalkyl;
R₆ and R₇ are each independently C₁₋₄alkyl or haloalkyl; and
R₈ is H, —COC₁-C₄alkyl (eg Ac), —COhydroxyalkyl or —COhaloalkyl; and
pharmaceutically acceptable esters, salts, and prodrugs thereof.

When R₃ and R₄ are taken together with C₂₀ to form C₃-C₆ cycloalkyl an example is cyclopropyl.

R₈ may typically represent H or Ac

In one embodiment, A₁ is a single bond and A₂ is a single bond, E or Z double bond, or a triple bond. In another embodiment, A₁ is a double bond and A₂ is a single bond, E or Z double bond, or a triple bond. One of ordinary skill in the art will readily appreciate that when A₂ is a triple bond, R₅ is absent.

In one embodiment, X₁ and X₂ are each H. In another embodiment, X₁ is CH₂ and X₂ is H₂.

In another embodiment, R₃ is hydrogen and R₄ is C₁-C₄ alkyl. In a preferred embodiment R₄ is methyl.

In another example set of compounds R₁ and R₂ both represent OAc.

In one set of example compounds R₆ and R₇ are each independently C₁₋₄-alkyl. In another set of example compounds R₆ and R₇ are each independently haloalkyl. In another embodiment, R₆ and R₇ are each independently methyl, ethyl or fluoroalkyl. In a preferred embodiment, R₆ and R₇ are each trifluoroalkyl, e.g., trifluoromethyl.

Typically R₅ represents hydrogen.

Thus, in certain embodiments, vitamin D compounds for use in accordance with the invention are represented by I-a: 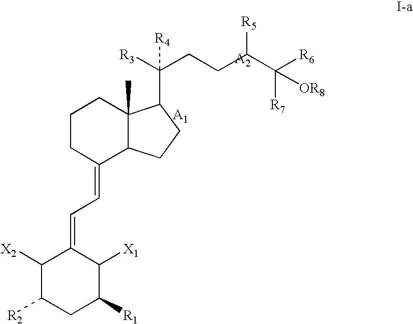
wherein:

A₁ is single or double bond;

A₂ is a single, double or triple bond;

X₁ and X₂ are each independently H or ═CH₂, provided X₁ and X₂ are not both ═CH₂;

R₁ and R₂ are each independently OC(O)C₁-C₄ alkyl, OC(O)hydroxyalkyl, or C(O)haloalkyl;

R₃, R₄ and R₅ are each independently hydrogen, C₁-C₄ alkyl, hydroxyalkyl, or haloalkyl, or R₃ and R₄ taken together with C₂₀ form C₃-C₆ cylcoalkyl;

R₆ and R₇ are each independently haloalkyl; and

R₈ is H, C(O)C₁-C₄ alkyl, C(O)hydroxyalkyl, or C(O)haloalkyl; and pharmaceutically acceptable esters, salts, and prodrugs thereof.

An example compound of the above-described formula I-a is
1,3-di-O-acetyl-1,25-dihydroxy-16,23Z-diene-26,27-hexafluoro-19-nor-cholecalciferol (“Compound C”):

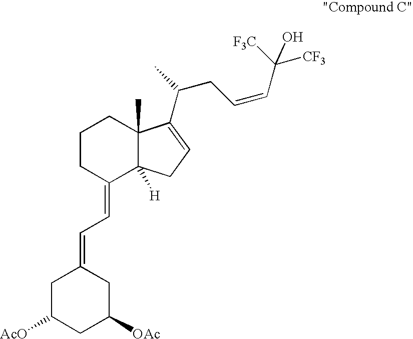

In another preferred embodiment, R₁ and R₂ are each OAc; A₁ is a double bond; A₂ is a triple bond; and R₈ is either H or Ac for example the following compound:

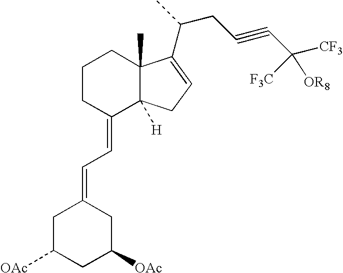

In certain embodiments of the above-represented formula I, vitamin D compounds for use in accordance with the invention are represented by the formula I-b:

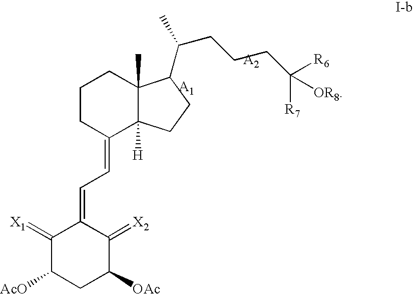

Other example compounds of the above-described formula I-b include:
1,3-di-O-acetyl-1,25-dihydroxy-23-yne-cholecalciferol;
1,3-di-O-acetyl-1,25-dihydroxy-16-ene-23-yne-cholecalciferol;
1,3-di-O-acetyl-1,25-dihydroxy-16,23E-diene-cholecalciferol;
1,3-di-O-acetyl-1,25-dihydroxy-16-ene-cholecalciferol;
1,3,25-Tri-O-acetyl-1,25-dihydroxy-16-ene-23-yne-26,27-hexafluoro-cholecalciferol:
1,3-di-O-acetyl-1,25-dihydroxy-16-ene-23-yne-26,27-hexafluoro-cholecalciferol;
1,3-Di-O-acetyl-1,25-dihydroxy-16,23E-diene-25R-26-trifluoro-cholecalciferol:
1,3-Di-O-acetyl-1,25-Dihydroxy-16-ene-23-yne-26,27-hexafluoro-19-nor-cholecalciferol:
1,3,25-Tri-O-acetyl-1,25-Dihydroxy-16-ene-23-yne-26,27-hexafluoro-19-nor-cholecalciferol:
1,3-di-O-acetyl-1,25-dihydroxy-16-ene-19-nor-cholecalciferol;
1,3-Di-O-acetyl-1,25-dihydroxy-16-ene-23-yne-19-nor-cholecalciferol:
1,3-Di-O-acetyl-1,25-dihydroxy-20-cyclopropyl-23-yne-19-nor-cholecalciferol:
1,3-Di-O-acetyl-1,25-dihydroxy-16-ene-23-yne-26,27-bishomo-19-nor-cholecalciferol;

In certain other embodiments of the above-represented formula I, the vitamin D compounds for use in accordance with the invention are represented by the formula I-c:

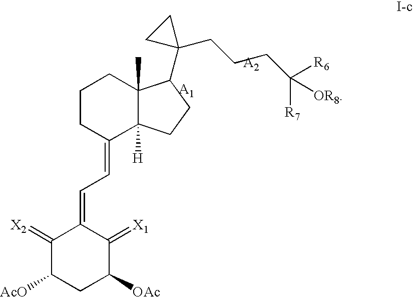

Other example compounds of the above-described formula I-b include:
1,3,25-tri-O-acetyl-1,25-dihydroxy-20-cyclopropyl-23-yne-26,27-hexafluoro-19-nor-cholecalciferol;
1,3-di-O-acetyl-1,25-dihydroxy-20-cyclopropyl-23-yne-26,27-hexafluoro-19-nor-cholecalciferol;
1,3-di-O-acetyl-1,25-dihydroxy-20-cyclopropyl-23-yne-cholecalciferol;
1,3-di-O-acetyl-1,25-dihydroxy-20-cyclopropyl-23E-ene-26,27-hexafluoro-19-nor-cholecalciferol;
1,3-di-O-acetyl-1,25-dihydroxy-20-cyclopropyl-23Z-ene-26,27-hexafluoro-19-nor-cholecalciferol;
1,3-di-O-acetyl-1,25-dihydroxy-20-cyclopropyl-cholecalciferol;
1,3-di-O-acetyl-1,25-dihydroxy-16-ene-20-cyclopropyl-19-nor-cholecalciferol; and
1,3-Dd-O-acetyl-1,25-dihydroxy-16-ene-20-cyclopropyl-cholecalciferol;

In another preferred embodiment, vitamin D compounds for use in accordance with the invention are compounds of the formula: 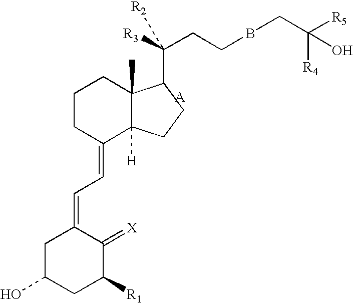
wherein
X is H₂ or CH₂;
R₁ is hydrogen, hydroxy or fluorine;
R₂ is hydrogen or methyl;
R₃ is hydrogen or methyl, when R₂ or R₃ is methyl, R₃ or R₂ must be hydrogen;
R₄ is methyl, ethyl or trifluoromethyl;
R₅ is methyl, ethyl or trifluoromethyl;
A is a single or double bond; and
B is a single, E-double, Z-double or triple bond.

In particularly preferred compounds, each of R₄ and R₅ is methyl or ethyl, for example 1-alpha-fluoro-25-hydroxy-16,23E-diene-26,27-bishomo-20-epi-cholecalciferol (Compound A in the following examples), having the formula:

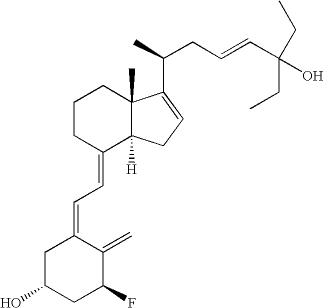

Such compounds are described in U.S. Pat. No. 5,939,408 and EP808833, the contents of which are herein incorporated by reference in their entirety. The invention also embraces use of esters and salts of Compound A. Esters include pharmaceutically acceptable labile esters that may be hydrolysed in the body to release Compound A. Salts of Compound A include adducts and complexes that may be formed with alkali and alkaline earth metal ions and metal ion salts such as sodium, potassium and calcium ions and salts thereof such as calcium chloride, calcium malonate and the like. However, although Compound A may be administered as a pharmaceutically acceptable salt or ester thereof, preferably Compound A is employed as is i.e., it is not employed as an ester or a salt thereof.

Other preferred vitamin D compounds for use in accordance with the invention included those having formula I-a: 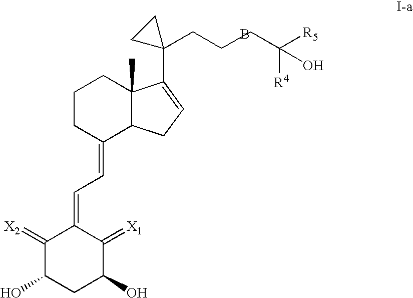
wherein:

B is single, double, or triple bond;

X₁ and X₂ are each independently H₂ or CH₂, provided X₁ and X₂ are not both CH₂; and

R₄ and R₅ are each independently alkyl or haloalkyl.

Compounds of formula I-a including the following:
1,25-Dihydroxy-16-ene-23-yne-20-cyclopyl-cholecalciferol: 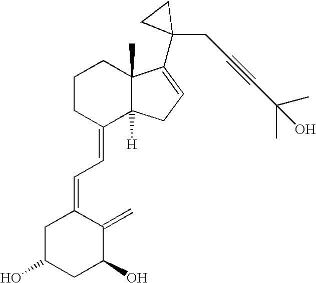
1,25-Dihydroxy-16-ene-23-yne-20-cyclopropyl-19-nor-cholecalciferol: 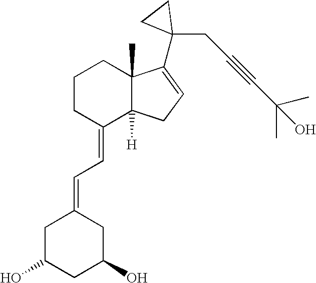
1,25-Dihydroxy-16-ene-20-cyclopropyl-23-yne-26,27-hexafluoro-19-nor-cholecalciferol: 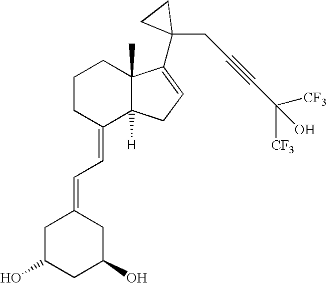
1,25-Dihydroxy-116-ene-20-cyclopropyl-23-yne-26,27-hexafluoro-cholecalciferol: 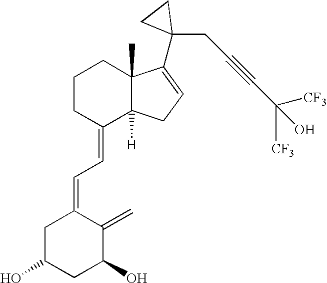
1,25-Dihydroxy-116,23E-diene-20-cyclopropyl-26,27-hexafluoro-19-nor-cholecalciferol: 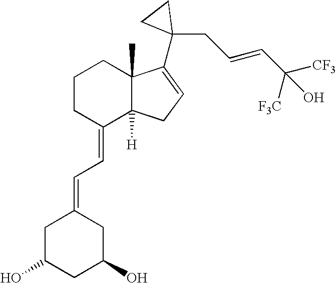
1,25-Dihydroxy-16,23E-diene-20-cyclopropyl-26,27-hexafluoro-cholecalciferol: 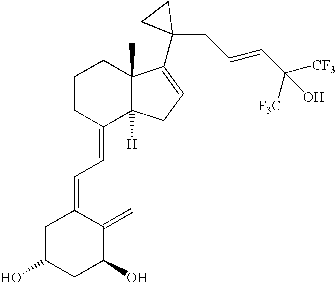
1,25-Dihydroxy-16,23Z-diene-20-cyclopropyl-26,27-hexafluoro-19-nor-cholecalciferol: 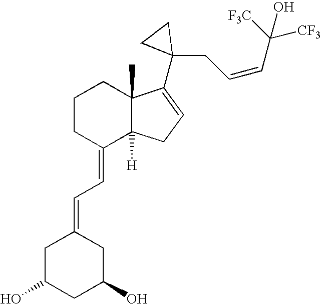
1,25-Dihydroxy-16,23Z-diene-20-cyclopropyl-26,27-hexafluoro-cholecalciferol: 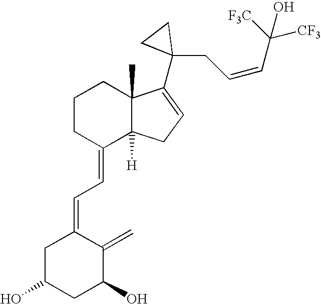
1,25-Dihydroxy-16-ene-20-cyclopropyl-19-nor-cholecalciferol: 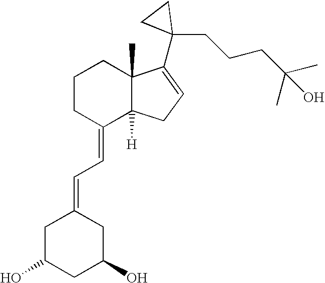
1,25-Dihydroxy-16-ene-20-cyclopropyl-cholecalciferol: 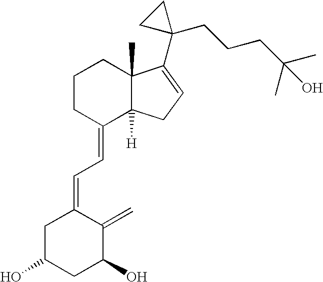
   Another vitamin D compounds of the invention is 1,25-dihydroxy-21(3-hydroxy-3-trifluoromethyl-4-trifluoro-butynyl)-26,27-hexadeutero-19-nor-20S-cholecalciferol.

The use of compounds having the structures given above is extended to pharmaceutically acceptable esters, salts, and prodrugs thereof. Examples are given in the previous paragraph.

A vitamin D compound of particular interest is calcitriol.

Other example compounds of use in the invention which are vitamin D receptor agonists include paricalcitol (ZEMPLAR™) (see U.S. Pat. No. 5,587,497), tacalcitol (BONALFA™) (see U.S. Pat. No. 4,022,891), doxercalciferol (HECTOROL™) (see Lam et al. (1974) Science 186, 1038), maxacalcitol (OXAROL™) (see U.S. Pat. No. 4,891,364), calcipotriol (DAIVONEX™) (see U.S. Pat. No. 4,866,048), and falecalcitriol (FULSTAN™).

Other compounds include ecalcidene, calcithiazol and tisocalcitate.

Other compounds include atocalcitol, lexacalcitol and seocalcitol.

Another compound of possible interest is secalciferol (“OSTEO D”).

Other non-limiting examples of vitamin D compounds that may be of use in accordance with the invention include those described in published international applications: WO 01/40177, WO0010548, WO0061776, WO0064869, WO0064870, WO0066548, WO0104089, WO0116099, WO0130751, WO0140177, WO0151464, WO0156982, WO0162723, WO0174765, WO0174766, WO0179166, WO0190061, WO0192221, WO0196293, WO02066424, WO0212182, WO0214268, WO03004036, WO03027065, WO03055854, WO03088977, WO04037781, WO04067504, WO8000339, WO8500819, WO8505622, WO8602078, WO8604333, WO8700834, WO8910351, WO9009991, WO9009992, WO9010620, WO9100271, WO9100855, WO9109841, WO9112239, WO9112240, WO9115475, WO9203414, WO9309093, WO9319044, WO9401398, WO9407851, WO9407852, WO9408958, WO9410139, WO9414766, WO9502577, WO9503273, WO9512575, WO9527697, WO9616035, WO9616036, WO9622973, WO9711053, WO9720811, WO9737972, WO9746522, WO9818759, WO9824762, WO9828266, WO9841500, WO9841501, WO9849138, WO9851663, WO9851664, WO9851678, WO9903829, WO9912894, WO9915499, WO9918070, WO9943645, WO9952863, those described in: U.S. Pat. No. 3,856,780, U.S. Pat. No. 3,994,878, U.S. Pat. No. 4,021,423, U.S. Pat. No. 4,026,882, U.S. Pat. No. 4,028,349, U.S. Pat. No. 4,225,525, U.S. Pat. No. 4,613,594, U.S. Pat. No. 4,804,502, U.S. Pat. No. 4,898,855, U.S. Pat. No. 5,039,671, U.S. Pat. No. 5,087,619, U.S. Pat. No. 5,145,846, U.S. Pat. No. 5,247,123, U.S. Pat. No. 5,342,833, U.S. Pat. No. 5,428,029, U.S. Pat. No. 5,451,574, U.S. Pat. No. 5,612,328, U.S. Pat. No. 5,747,479, U.S. Pat. No. 5,804,574, U.S. Pat. No. 5,811,414, U.S. Pat. No. 5,856,317, U.S. Pat. No. 5,872,113, U.S. Pat. No. 5,888,994, U.S. Pat. No. 5,939,408, U.S. Pat. No. 5,962,707, U.S. Pat. No. 5,981,780, U.S. Pat. No. 6,017,908, U.S. Pat. No. 6,030,962, U.S. Pat. No. 6,040,461, U.S. Pat. No. 6,100,294, U.S. Pat. No. 6,121,312, U.S. Pat. No. 6,329,538, U.S. Pat. No. 6,331,642, U.S. Pat. No. 6,392,071, U.S. Pat. No. 6,452,028, U.S. Pat. No. 6,479,538, U.S. Pat. No. 6,492,353, U.S. Pat. No. 6,537,981, U.S. Pat. No. 6,544,969, U.S. Pat. No. 6,559,138, U.S. Pat. No. 6,667,298, U.S. Pat. No. 6,683,219, U.S. Pat. No. 6,696,431, U.S. Pat. No. 6,774,251, and those described in published US Patent Applications: US2001007907, US2003083319, US2003125309, US2003130241, US2003171605, US2004167105.

It will be noted that the structures of some of the compounds of the invention include asymmetric carbon atoms. Accordingly, it is to be understood that the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

The preferred stereochemistry of compounds is as represented absolutely by the structures disclosed herein.

Naturally occurring or synthetic isomers can be separated in several ways known in the art. Methods for separating a racemic mixture of two enantiomers include chromatography using a chiral stationary phase (see, e.g., “Chiral Liquid Chromatography,” W.J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

The invention also provides a pharmaceutical composition, comprising an effective amount of a vitamin D compound as described herein and a pharmaceutically acceptable carrier. In a further embodiment, the effective amount is effective to treat bladder dysfunction, as described previously.

In an embodiment, the vitamin D compound is administered to the subject using a pharmaceutically-acceptable formulation, e.g., a pharmaceutically-acceptable formulation that provides sustained delivery of the vitamin D compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those vitamin D compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a vitamin D compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect. Generally, out of one hundred percent, this amount will range from about 0.1 to about 99.5 percent e.g. from about 1 percent to about 99 percent of active ingredient or else from about 0.5 percent to about 90 percent, preferably from about 5 percent to about 70 percent, most preferably from about 10 percent to about 30 percent by weight.

Methods of preparing these compositions include the step of bringing into association a vitamin D compound(s) with the carrier and, optionally, one or more accessory ingredients. In general, the formulations are prepared by uniformly and intimately bringing into association a vitamin D compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that releases the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the vitamin D compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active vitamin D compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more vitamin D compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a vitamin D compound(s) include powders, sprays, ointments, pastes, creams, lotions, gels, solutions, patches and inhalants. The active vitamin D compound(s) may be mixed under sterile conditions with a pharmaceutically-acceptable carrier, and with any preservatives, buffers, or propellants which may be required.

The ointments, pastes, creams and gels may contain, in addition to vitamin D compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D compound(s), excipients such as lactose, talc, silicic acid, aluminium hydroxide, calcium silicates and polyamide powder, or mixtures of these substances. Sprays can additionally contain customary propellants, such as chlorofluorohydrocarbons or hydrofluoroalkanes such as HFA134a or HFA227 and volatile unsubstituted hydrocarbons, such as butane and propane.

The vitamin D compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Pharmaceutical compositions of the invention suitable for parenteral administration comprise one or more vitamin D compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of vitamin D compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Regardless of the route of administration selected, the vitamin D compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 300 ug per day. An exemplary dose range of Compound A is from 0.1 to 300 ug per day, for example 50-150 ug per day e.g., 75 or 150 ug per day. A unit dose formulation preferably contains 50-150 ug e.g., 75 or 150 ug and is preferably administered once per day.

A preferred dose of the vitamin D compound for the present invention is the maximum that a patient can tolerate and not develop hypercalcemia. Preferably, the vitamin D compound of the present invention is administered at a concentration of about 0.001 ug to about 100 ug per kilogram of body weight, about 0.001 to about 10 ug/kg or about 0.001 ug to about 100 ug/kg of body weight. Ranges intermediate to the above-recited values are also intended to be part of the invention.

The invention also includes a packaged formulation including a pharmaceutical composition comprising a vitamin D compound and a pharmaceutically acceptable carrier packaged with instructions for use in the prevention and/or treatment of bladder dysfunction.

Composition of use according to the invention may include the vitamin D compound in combination with another substance suitable for treatment of prevention of bladder dysfunction e.g., an anti-muscarinic agent and/or an alpha blocker.

II. Synthesis of Compounds

A number of the compounds for use in the present invention can be prepared by incubation of vitamin D₃ analogues in cells, for example, incubation of vitamin D₃ analogues in either UMR 106 cells or Ros 17/2.8 cells results in production of vitamin D₃ compounds for use in the invention. For example, incubation of 1,25-dihydroxy-16-ene-5,6-trans-calcitriol in UMR 106 cells results in production of 1,25-dihydroxy-16-ene-24-oxo-5,6-trans-calcitriol.

In addition to the methods described herein, compounds of the present invention can be prepared using a variety of synthetic methods. For example, one skilled in the art would be able to use methods for synthesizing existing vitamin D₃ compounds to prepare compounds of use in the invention (see e.g., Bouillon, R. et al. (1995) Endocr. Rev. 16(2):200-257; Ikekawa, N. (1987) Med. Res. Rev. 7:333-366; DeLuca, H. F. and Ostrem, V. K. (1988) Prog. Clin. Biol. Res. 259:41-55; Ikekawa, N. and Ishizuka, S. (1992) CRC Press 8:293-316; Calverley, M. J. and Jones, G. (1992) Academic Press 193-270; Pardo, R. and Santelli, M. (1985) Bull. Soc. Chim. Fr:98-114; Bythgoe, B. (1980) Chem. Soc. Rev. 449-475; Quinkert, G. (1985) Synform 3:41-122; Quinkert, G. (1986) Synform 4:131-256; Quinkert, G. (1987) Synform 5:1-85; Mathieu, C. et al. (1994) Diabetologia 37:552-558; Dai, H. and Posner, G. H. (1994) Synthesis 1383-1398; and DeLuca, et al., WO 97/11053).

Exemplary methods of synthesis include the photochemical ring opening of a 1-hydroxylated side chain-modified derivative of 7-dehydrocholesterol which initially produces a previtamin that is easily thermolyzed to vitamin D₃ in a well known fashion (Barton, D. H. R. et al. (1973) J. Am. Chem. Soc. 95:2748-2749; Barton, D. H. R. (1974) JCS Chem. Comm. 203-204); phosphine oxide coupling method developed by (Lythgoe, et al. (1978) JCS Perkin Trans. 1:590-595) which comprises coupling a phosphine oxide to a Grundmann's ketone derivative to directly produce a 1-alpha,25(OH)₂D₃ skeleton as described in Baggiolini, E. (1986) J. Org. Chem. 51:3098-3108; DeSchrijver, J. and DeClercq, P. J. (1993) Tetrahed Lett 34:4369-4372; Posner, G. H and Kinter, C. M. (1990) J. Org. Chem. 55:3967-3969; semihydrogenation of dienynes to a previtamin structure that undergoes rearrangement to the corresponding vitamin D₃ analogue as described by Harrison, R. G. et al. (1974) JCS Perkin Trans. 1:2654-2657; Castedo, L. et al. (1988) Tetrahed Lett 29:1203-1206; Mascarenas, J. S. (1991) Tetrahedron 47:3485-3498; Barrack, S. A. et al. (1988) J. Org. Chem. 53:1790-1796) and Okamura, W. H. et al. (1989) J. Org Chem. 54:4072-4083; the vinylallene approach involving intermediates that are subsequently arranged using heat or a combination of metal catalyzed isomerization followed by sensitized photoisomerization (Okamura, W. H. et al. (1989) J. Org. Chem. 54:4072-4083; Van Alstyne, E. M. et al. (1994) J. Am. Chem. Soc. 116:6207-6210); the method described by Trost, B. M. et al. J. Am. Chem. Soc. 114:9836-9845; Nagasawa, K. et al. (1991) Tetrahed Lett 32:4937-4940 involves an acyclic A-ring precursor which is intramolecular cross-coupled to the bromoenyne leading directly to the formation of 1,25(OH)₂D₃ skeleton; a tosylated derivative which is isomerized to the i-steroid that can be modified at carbon-1 and then subsequently back-isomerized under sovolytic conditions to form 1-alpha,25(OH)₂D₂ or analogues thereof (Sheves, M. and Mazur, Y. (1974) J. Am. Chem. Soc. 97:6249-6250; Paaren, H. E. et al. (1980) J. Org. Chem. 45:3253-3258; Kabat, M. et al. (1991) Tetrahed Lett 32:2343-2346; Wilson, S. R. et al. (1991) Tetrahed Lett 32:2339-2342); the direct modification of vitamin D derivatives to 1-oxygenated 5,6-trans vitamin D as described in (Andrews, D. R. et al. (1986) J. Org. Chem. 51:1635-1637); the Diels-Alders cycloadduct method of previtamin D₃ can be used to cyclorevert to 1-alpha,25(OH)₂D₂ through the intermediary of a previtamin form via thermal isomerization (Vanmaele, L. et al. (1985) Tetrahedron 41:141-144); and, a final method entails the direct modification of 1-alpha,25(OH)₂D₂ or an analogue through use of suitable protecting groups such as transition metal derivatives or by other chemical transformations (Okarmura, W. H. et al. (1992) J. Cell Biochem. 49:10-18). Additional methods for synthesizing vitamin D₂ compounds are described in, for example, Japanese Patent Disclosures Nos. 62750/73, 26858176, 26859/76, and 71456177; U.S. Pat. Nos. 3,639,596; 3,715,374; 3,847,955 and 3,739,001.

Examples of the compounds of use in this invention having a saturated side chain can be prepared according to the general process illustrated and described in U.S. Pat. No. 4,927,815. Examples of compounds of the invention having an unsaturated side chain can be prepared according to the general process illustrated and described in U.S. Pat. No. 4,847,012. Examples of compounds of the invention wherein R groups at position C20 together represent a cycloalkyl group can be prepared according to the general process illustrated and described in U.S. Pat. No. 4,851,401.

Another synthetic strategy for the preparation of side-chain-modified analogues of 1-alpha,25-dihydroxyergocalciferol is disclosed in Kutner et al., The Journal of Organic Chemistry, 1988, 53:3450-3457. In addition, the preparation of 24-homo and 26-homo vitamin D analogues are disclosed in U.S. Pat. No. 4,717,721.

The enantioselective synthesis of chiral molecules is now state of the art. Through combinations of enantioselective synthesis and purification techniques, many chiral molecules can be synthesized as an enantiomerically enriched preparation. For example, methods have been reported for the enantioselective synthesis of A-ring diastereomers of 1-alpha,25(OH)₂D₃ as described in Muralidharan et al. (1993) J. Organic Chem. 58(7): 1895-1899 and Norman et al. (1993) J. Biol. Chem. 268(27): 20022-30. Other methods for the enantiomeric synthesis of various compounds known in the art include, inter alia, epoxides (see, e.g., Johnson, R. A.; Sharpless, K. B. in Catalytic Asymmetric Synthesis; Ojima, I., Ed.: VCH: New York, 1993; Chapter 4.1. Jacobsen, E. N. ibid. Chapter 4.2), diols (e.g., by the method of Sharpless, J. Org. Chem. (1992) 57:2768), and alcohols (e.g., by reduction of ketones, E. J. Corey et al., J. Am. Chem. Soc. (1987) 109:5551). Other reactions useful for generating optically enriched products include hydrogenation of olefins (e.g., M. Kitamura et al., J. Org. Chem. (1988) 53:708); Diels-Alder reactions (e.g., K. Narasaka et al., J. Am. Chem. Soc. (1989) 111:5340); aldol reactions and alkylation of enolates (see, e.g., D. A. Evans et al., J. Am. Chem. Soc. (1981) 103:2127; D. A. Evans et al., J. Am. Chem. Soc. (1982) 104:1737); carbonyl additions (e.g., R. Noyori, Angew. Chem. Int. Ed. Eng. (1991) 30:49); and ring-opening of meso-epoxides (e.g., Martinez, L. E.; Leighton J. L., Carsten, D. H.; Jacobsen, E. N. J. Am. Chem. Soc. (1995) 117:5897-5898). The use of enzymes to produce optically enriched products is also well known in the art (e.g., M. P. Scheider, ed. “Enzymes as Catalysts in Organic Synthesis”, D. Reidel, Dordrecht (1986).

Chiral synthesis can result in products of high stereoisomer purity. However, in some cases, the stereoisomer purity of the product is not sufficiently high. The skilled artisan will appreciate that the separation methods described herein can be used to further enhance the stereoisomer purity of the vitamin D₃-epimer obtained by chiral synthesis.

III. Examples of Chemical Synthesis of Certain Preferred Compounds

### EXPERIMENTAL

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 μm mesh silica gel. Preparative HPLC was performed on a 5×50 cm column and 15-30 μm mesh silica gel at a flow rate of 100 ml/min.

### Example 1

### Synthesis of 1,3-Di-O-acetyl-1,25-dihydroxy-16,23Z-diene-26,27-hexafluoro-19-nor-cholecalciferol(1)

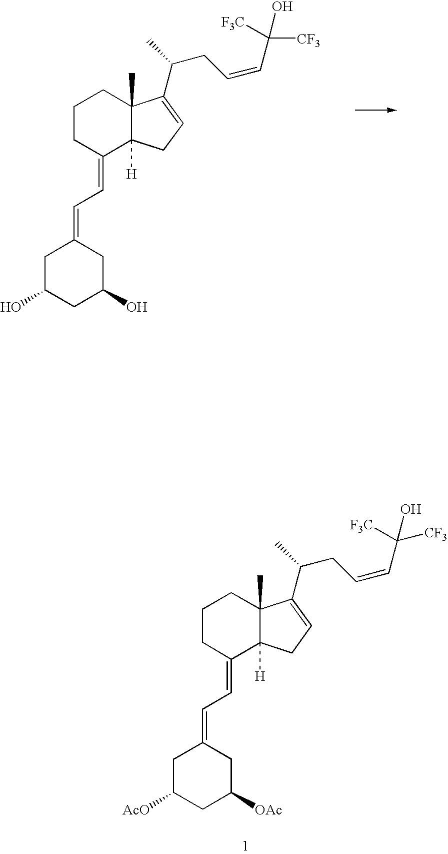 The starting material 1,25-dihydroxy-16,23Z-diene-26,27-hexafluoro-19-nor-cholecalciferol can be prepared as described in U.S. Pat. No. 5,428,029 to Doran et al. 3 mg of 1,25-dihydroxy-16,23Z-diene-26,27-hexafluoro-19-nor-cholecalciferol was dissolved in 0.8 ml of pyridine, cooled to ice-bath temperature and 0.2 ml of acetic anhydride was added and maintained at that temperature for 16 h. Then the reaction mixture was diluted with 1 ml of water, stirred for 10 min in the ice bath and distributed between 5 ml of water and 20 ml of ethyl acetate. The organic layer was washed with 3×5 ml of water, once with 5 ml of saturated sodium hydrogen carbonate, once with 3 ml of brine then dried (sodium sulfate) and evaporated. The oily residue was taken up in 1:6 ethyl acetate-hexane and flash-chromatographed using a stepwise gradient of 1:6, 1:4 and 1:2 ethyl acetate-hexane. The column chromatography was monitored by TLC (1:4 ethyl acetate-hexane, spot visualization with phosphomolybdic acid spray), the appropriate fractions were pooled, evaporated, the residue taken up in methyl formate, filtered, then evaporated again to give 23.8 mg of the title compound (1) as a colorless syrup; 400 MHz ¹H NMR δ 0.66 (3H, s), 0.90 (1H, m), 1.06 (3H, d, J=7.2 Hz), 1.51 (1H, m), 1.72-1.82 (3H, m), 1.9-2.1 (3H, m), 1.99 (3H, s) 2.04 (3H, s), 2.2-2.3 (3 m), 2.44-2.64 (6H, m), 2.78 (1H, m), 3.01 (1H, s), 5.10 (2H, m), 5.38 (1H, m), 5.43 (1H, d, J=12 Hz), 5.85 (1H, d, J=11.5 Hz), 5.97 (1H, dt, J=12 and 7.3 Hz), 6.25 (1H, d, J=11.5 Hz).

### Example 2

### Synthesis of 1,3-Di-O-acetyl-1,25-Dihydroxy-16-ene-23-yne-26,27-hexafluoro-19-nor-cholecalciferol (2) and 1,3,25-Tri-O-acetyl-1,25-Dihydroxy-16-ene-23-yne-26,27-hexafluoro-19-nor-cholecalciferol (3)

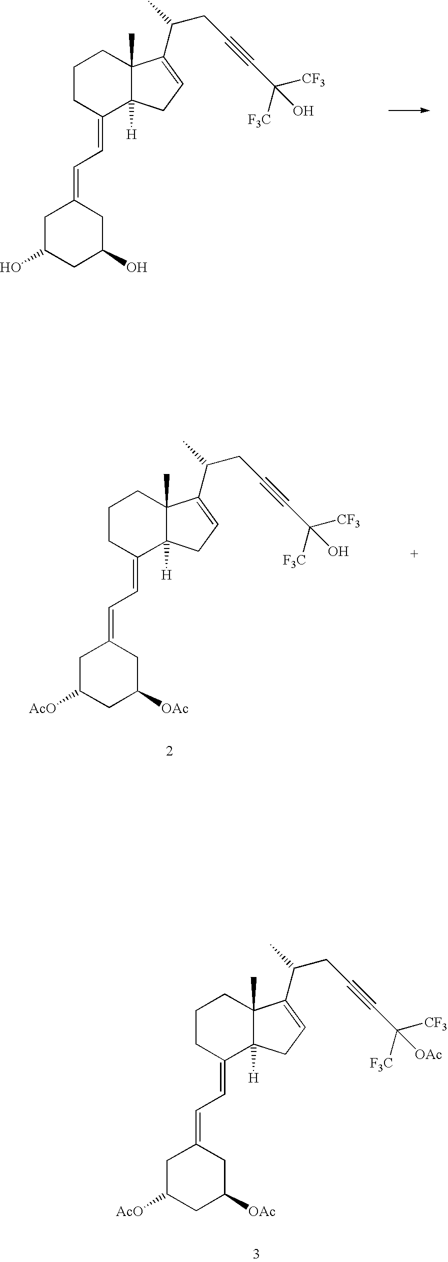 The starting material 1,25-dihydroxy-16-ene-23-yne-26,27-hexafluoro-19-nor-cholecalciferol can be prepared as described in U.S. Pat. Nos. 5,451,574 and 5,612,328 to Baggiolini et al. 314 mg (0.619 mmole) of 1,25-dihydroxy-16-ene-23-yne-26,27-hexafluoro-19-nor-cholecalciferol was dissolved in 1.5 ml of pyridine, cooled to ice-bath temperature, and 0.4 ml of acetic anhydride was added. The reaction mixture was kept at room temperature for 7 hours and then for 23 hours in a refrigerator. It was then diluted with 10 ml water and extracted with 30 ml of ethyl acetate. The organic extract was washed with water and brine, dried over sodium sulfate and evaporated. The residue was FLASH chromatographed on a 10×140 mm column with 1:6 and 1:4 ethyl acetate-hexane as the mobile phase to give 126 mg of 1,3-Di-O-acetyl-1,25-Dihydroxy-16-ene-23-yne-26,27-hexafluoro-19-nor-cholecalciferol (2), and 248 mg of 1,3,25-Tri-O-acetyl-1,25-Dihydroxy-16-ene-23-yne-26,27-hexafluoro-19-nor-cholecalciferol (3).

### Example 3

### Synthesis of 1,3-Di-O-acetyl-1,25-dihydroxy-16-ene-23-yne-cholecalciferol (4)

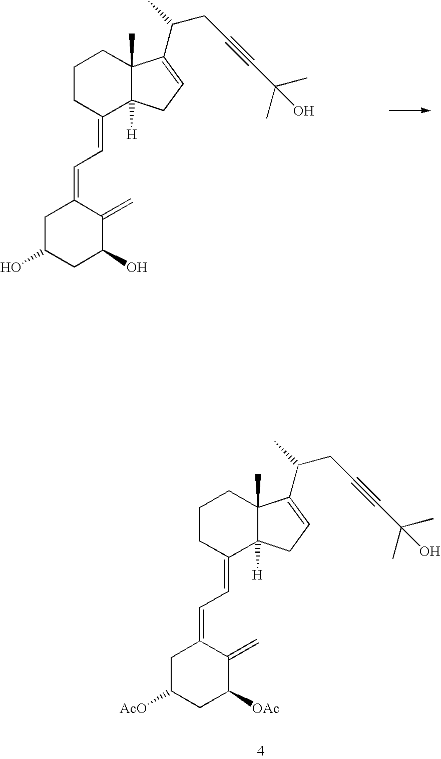

A 10-mL round-bottom flask was charged with 40 mg of 1,25-dihydroxy-16-ene-23-yne-cholecalciferol. This material was dissolved in 1 mL of pyridine. This solution was cooled in an ice bath then 0.3 mL of acetic anhydride was added. The solution was stirred for 30 min, then refrigerated overnight, diluted with water and transferred to a separatory funnel with the aid of 10 mL of water and 40 mL of ethyl acetate. The organic layer was washed with 4×20 mL of water, 10 mL of brine passed through a plug of sodium sulfate and evaporated. The light brown, oily residue was taken up in 1:9 ethyl acetate-hexane then flash chromatographed on a 10×130 mm column using 1:9 ethyl acetate-hexane as mobile phase for fractions 1-5, 1:6 for fractions 6-13 and 1:4 ethyl acetate-hexane for fractions 14-20 (18 mL fractions). Fractions 14-19 contained the main band with Rf0.15 (TLC 1:4). Those fractions were pooled and evaporated to a colorless oil, 0.044 g. The material was taken up in methyl formate, filtered and evaporated to give a colorless, sticky foam, 0.0414 g of the title compound (4).

### Example 4

### Synthesis of 1,3-Di-O-acetyl-1,25-dihydroxy-16,23E-diene-cholecalciferol (5)

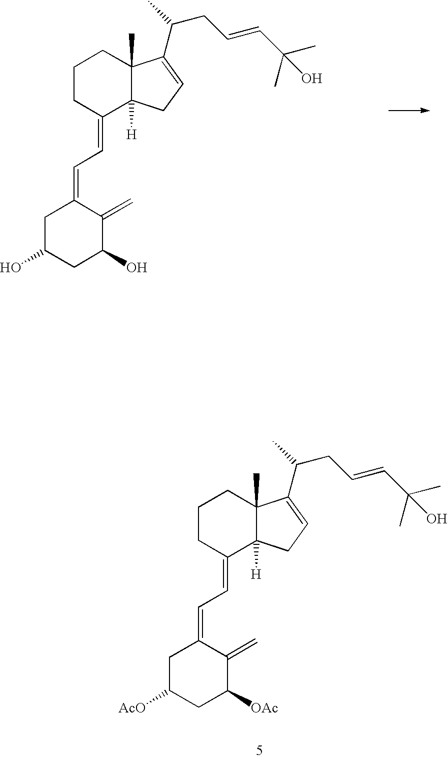

0.0468 g of 1,25-Dihydroxy-16,23E-diene-cholecalciferol was dissolved in 1.5 mL of pyridine. This solution was cooled in an ice bath then refrigerated overnight, diluted with 10 mL of water while still immersed in the ice bath, stirred for 10 min and transferred to a separatory funnel with the aid of 10 mL of water and 40 mL of ethyl acetate. The organic layer was washed with 4×20 mL of water, 10 mL of brine passed through a plug of sodium sulfate and evaporated. The light brown, oily residue was taken up in 1:9 ethyl acetate-hexane then flash chromatographed on a 10×130 mm column using 1:9 ethyl acetate-hexane as mobile phase for fractions 1-3 (20 mL fractions), 1:6 for fractions 6-8 and 1:4 ethyl acetate-hexane for fractions 9-17 (18 mL each). Fractions 11-14 contained the main band with Rf 0.09 (TLC 1:4). Those fractions were pooled and evaporated to a colorless oil, 0.0153 g. This material was taken up in methyl formate, filtered and evaporated, to give 0.014 g of the title compound (5).

### Example 5

### Synthesis of 1,3-Di-O-acetyl-1,25-dihydroxy-16-ene-cholecalciferol (6)

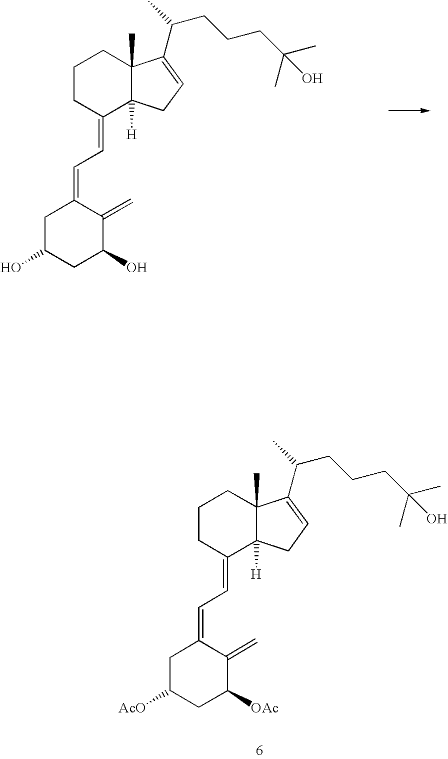

0.0774 g of 1,25-Dihydroxy-16-ene-cholecalciferol was dissolved in 1.5 mL of pyridine. This solution was cooled in an ice bath then 0.3 mL of acetic anhydride was added. The solution was stirred, refrigerated overnight then diluted with 1 mL of water, stirred for 1 h in the ice bath and diluted with 30 mL of ethyl acetate and 15 mL of water. The organic layer was washed with 4×15 mL of water, once with 5 mL of brine then dried (sodium sulfate) and evaporated. The light brown, oily residue was taken up in 1:9 ethyl acetate-hexane then flash chromatographed on a 10×130 mm column using 1:9 ethyl acetate-hexane as mobile phase for fraction 1 (20 mL fractions), 1:6 for fractions 2-7 and 1:4 ethyl acetate-hexane for fractions 8-13. Fractions 9-11 contained the main band with Rf 0.09 (TLC 1:4 ethyl acetate-hexane). Those fractions were pooled and evaporated to a colorless oil, 0.0354 g. This material was taken up in methyl formate, filtered and the solution evaporated, 0.027 g colorless film, the title compound (6).

### Example 6

### Synthesis of 1,3,25-Tri-O-acetyl-1,25-dihydroxy-16-ene-23-yne-26,27-hexafluoro-cholecalciferol (7) and 1,3-Di-O-acetyl-1,25-dihydroxy-16-ene-23-yne-26,27-hexafluoro-cholecalciferol (8)

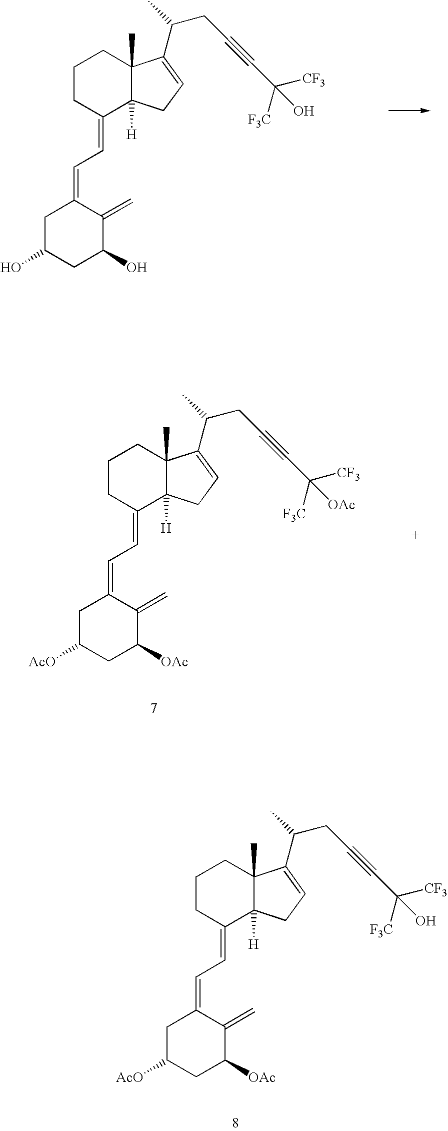

0.0291 g of 1,25-dihydroxy-16-ene-23-yne-26,27-hexafluoro-cholecalciferol was dissolved in 1.5 mL of pyridine. This solution was cooled in an ice bath then 0.25 mL of acetic anhydride was added. The solution was stirred for 20 min and kept in a freezer overnight. The cold solution was diluted with 15 mL of water, stirred for 10 min, and diluted with 30 mL of ethyl acetate. The organic layer was washed with 4×15 mL of water, once with 5 mL of brine then dried (sodium sulfate) and evaporated. The light brown, oily residue was taken up in 1:6 ethyl acetate-hexane then flash chromatographed on a 10×110 mm column using 1:6 ethyl acetate-hexane as mobile phase. Fractions 2-3 gave 72.3461-72.3285=0.0176 g. Evaporation of fractions 6-7 gave 0.0055 g. The residue of fractions 2-3 was taken up in methyl formate, filtered and evaporated to give 0.0107 g of the title triacetate (7). The residue of fractions 6-7 was taken up in methyl formate, filtered and evaporated to give 0.0049 g of diacetate (8).

### Example 7

### Synthesis of 1,3-Di-O-acetyl-1,25-dihydroxy-16,23E-diene-25R,26-trifluoro cholecalciferol (9)

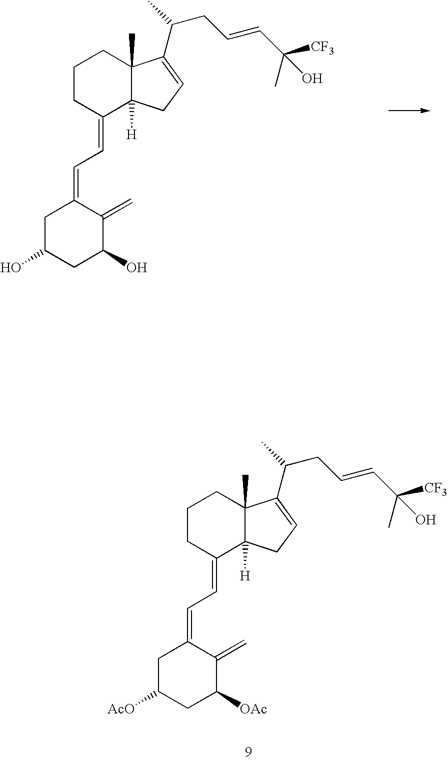

1.5 mL of 1,25-dihydroxy-16,23E-diene-25R,26-trifluoro-cholecalciferol was dissolved in 1.5 mL of pyridine, cooled to ice-bath temperature and 0.4 mL of acetic anhydride was added. The mixture was then refrigerated. After two days the mixture was diluted with 1 mL of water, stirred for 10 min in the ice bath then distributed between 10 mL of water and 30 mL of ethyl acetate. The organic layer was washed with 4×15 mL of water, once with 5 mL of brine then dried (sodium sulfate) and evaporated. The light brown, oily residue was taken up in 1:6 ethyl acetate-hexane then flash chromatographed on a 10×130 mm column using 1:6 ethyl acetate-hexane as mobile phase. Fractions 4-6 (TLC, 1:4) contained the main band (see TLC) These fractions were evaporated and gave 0.0726 g. This residue was taken up in methyl formate, filtered and evaporated, to give 0.0649 g of colorless foam, the title compound (9).

### Example 8

### Synthesis of 1,3-Di-O-acetyl-1,25-dihydroxy-16-ene-19-nor-cholecalciferol (10)

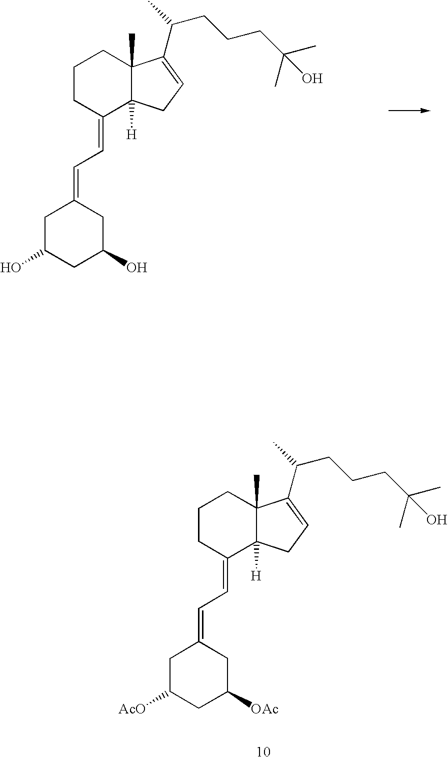

0.0535 g of 1,25-Dihydroxy-16-ene-19-nor-cholecalciferol was dissolved in 1.5 mL of pyridine, cooled to ice-bath temperature and 0.3 mL of acetic anhydride was added and the mixture was refrigerated overnight. The solution was diluted with 1 mL of water, stirred for 10 min in the ice bath then distributed between 10 mL of water and 30 mL of ethyl acetate. The organic layer was washed with 4×15 mL of water, once with 5 mL of brine then dried (sodium sulfate) and evaporated. The nearly colorless, oily residue was taken up in 1:6 ethyl acetate-hexane as mobile phase for fractions 1-6 then 1:4 ethyl acetate-hexane was used. Fractions 9-19 (TLC, 1:4 ethyl acetate-hexane, Rf 0.09, see below) were pooled, evaporated, to give 0.0306 g, which was taken up in methyl formate, filtered, then evaporated. It gave 0.0376 of the title compound (10).

### Example 9

### Synthesis of 1,3-Di-O-Acetyl-1,25-dihydroxy-16-ene-23-yne-19-nor-cholecalciferol (11)

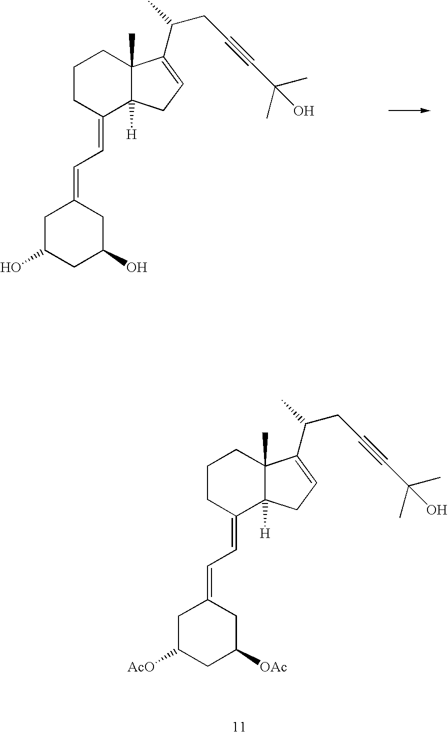

50 mg of 1,25-dihydroxy-16-ene-23-yne-19-nor-cholecalciferol was dissolved in 0.8 mL of pyridine, cooled to ice-bath temperature and 0.2 mL of acetic anhydride was added. The mixture was refrigerated for 3 days then diluted with 1 mL of water, stirred for 10 min in the ice bath and distributed between 5 mL of water and 20 mL of ethyl acetate. The organic layer was washed with 4×5 mL of water, once with 3 mL of brine then dried (sodium sulfate) and evaporated. The nearly colorless, oily residue was taken up in 1:6 ethyl acetate-hexane then flash chromatographed on a 15×120 mm column using 1:6 ethyl acetate-hexane as mobile phase for fractions 1-6, 1:4 for fractions 9-12, 1:3 for fractions 13-15 and 1:2 ethyl acetate-hexane for the remaining fractions. Fractions 11-16 (TLC, 1:4 ethyl acetate-hexane, Rf 0.09, see below) were pooled, evaporated 76.1487-76.1260=0.0227 g, taken up in methyl formate, filtered, then evaporated. It gave 0.0186 g of the title compound (11).

### Example 10

### Synthesis of 1,3-Di-O-acetyl-1,25-dihydroxy-16-ene-23-yne-26,27-bishomo-19-nor-cholecalciferol (12)

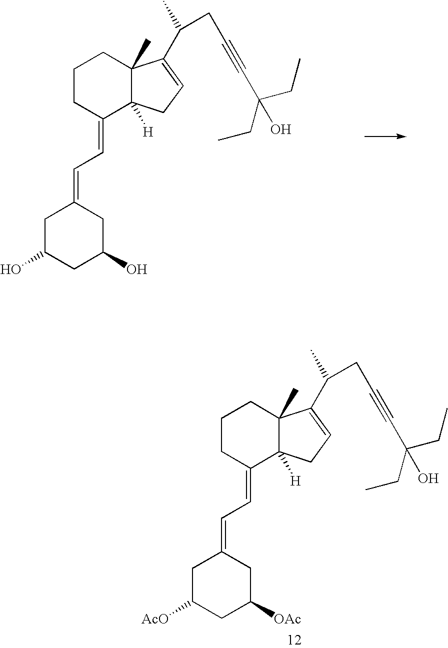

0.0726 g of 1,25-dihydroxy-16-ene-23-yne-26,27-bishomo-19-nor-cholecalciferol was dissolved in 0.8 mL of pyridine, cooled to ice-bath temperature and 0.2 mL of acetic anhydride was added. The solution was stirred in the ice-bath then refrigerated overnight. The solution was then diluted with 1 mL of water, stirred for 10 min in the ice bath and distributed between 10 mL of water and 25 mL of ethyl acetate. The organic layer was washed with 3×10 mL of water, once with 5 mL of saturated sodium hydrogen carbonate, once with 3 mL of brine then dried and evaporated, 33.5512−33.4654=0.0858 g of a tan oily residue that was flash-chromatographed on a 15×120 mm column using 1:6 as mobile phase. Fractions 7-11 (20 mL each) were pooled (TLC 1:4 ethyl acetate-hexane, Rf 0.14) and evaporated, 67.2834-67.2654=0.018 g. This residue was taken up in methyl formate, filtered and evaporated. It gave 0.0211 g of the title compound (12).

### Example 11

### Synthesis of 1,3-Di-O-acetyl-1,25-dihydroxy-20-cyclopropyl-23-yne-19-nor-cholecalciferol (13)

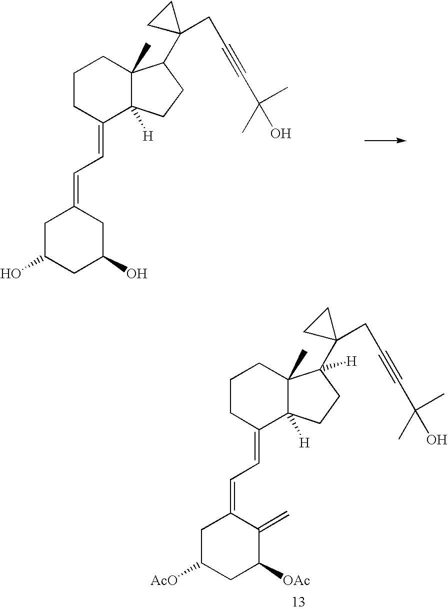

0.282 g of 1,25-Dihydroxy-20-cyclopropyl-23-yne-19-nor-cholecalciferol was dissolved in 0.8 mL of pyridine, cooled to ice-bath temperature and 0.2 mL of acetic anhydride was added and the mixture was refrigerated overnight, then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash chromatographed on a 15×110 mm column using 1:6 ethyl acetate-hexane as mobile phase for fractions 14, 1:4 for fractions 5-12, 1:3 for fractions 13-15 ethyl acetate-hexane for the remaining fractions. Fractions 7-12 (TLC, 1:4 ethyl acetate-hexane, Rf 0.13) were pooled, evaporated, the residue taken up in methyl formate, filtered, then evaporated to give 0.023 g of the title compound (13).

### Example 12

### Synthesis of 1,3,25-Tri-O-acetyl-1,25-dihydroxy-20-cyclopropyl-23-yne-26,27-hexafluoro-19-nor-cholecalciferol (14) and 1,3-Di-O-acetyl-1,25-dihydroxy-20-cyclopropyl-23-yne-26,27-hexafluoro-19-nor-cholecalciferol (15)

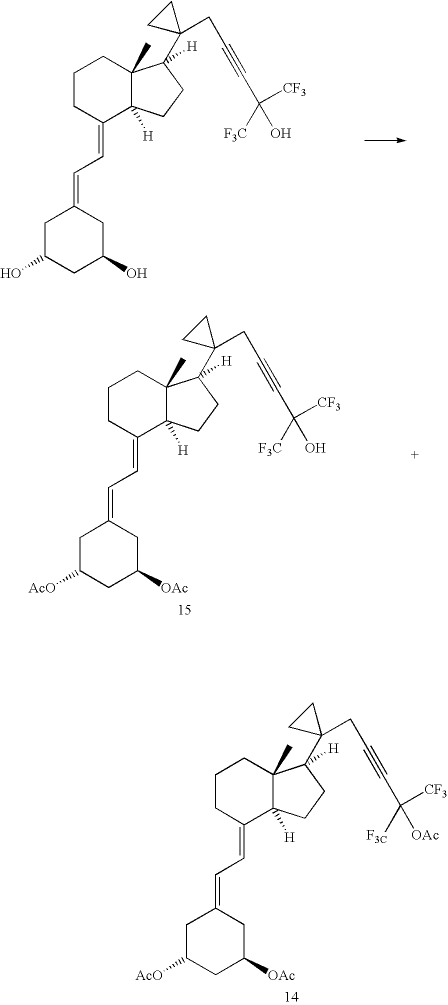

0.1503 g of 1,25-dihydroxy-20-cyclopropyl-23-yne-26,27-hexafluoro-19-nor-cholecalciferol was dissolved in 0.8 mL of pyridine, cooled to ice-bath temperature and 0.2 mL of acetic anhydride was added. The mixture was refrigerated overnight then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash chromatographed on a 15×150 mm column using 1:6 ethyl acetate-hexane as mobile phase for fractions 1-5, 1:4 for the remaining fractions. Fractions 3-4 and 6-7 were pooled, evaporated, then taken up in methyl formate, filtered, and evaporated to give 0.0476 g of the title triacetate (14) and 0.04670 g of the title diacetate (15).

### Example 13

### Synthesis of 1,3-Di-O-acetyl-1,25-dihydroxy-20-cyclopropyl-23-yne-cholecalciferol (16)

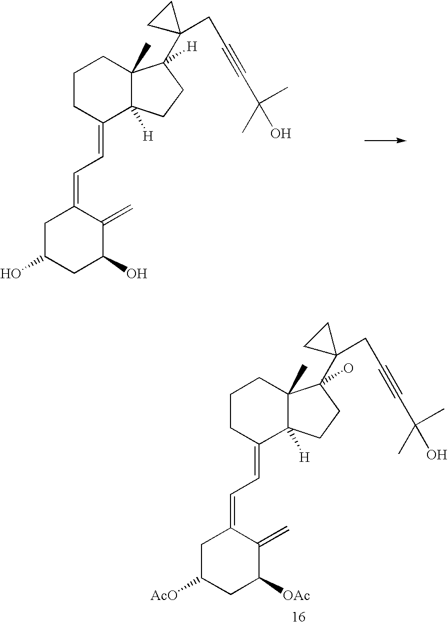

0.0369 g of 1,25-dihydroxy-20-cyclopropyl-23-yne-cholecalciferol was dissolved in 0.8 mL of pyridine, cooled to ice-bath temperature and 0.2 mL of acetic anhydride was added and the mixture was refrigerated overnight, then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash-chromatographed on a 13×110 mm column using 1:6 ethyl acetate-hexane as mobile phase for fractions 1-7, 1:4 ethyl acetate-hexane for the remaining fractions. Fractions 9-11 (TLC, 1:4 ethyl acetate-hexane) were pooled, evaporated, taken up in methyl formate, filtered, then evaporated, to give 0.0099 g of the title compound (16).

### Example 14

### Synthesis of 1,3-Di-O-acetyl-1,25-dihydroxy-20-cyclopropyl-23E-ene-26,27-hexafluoro-19-nor-cholecalciferol (17)

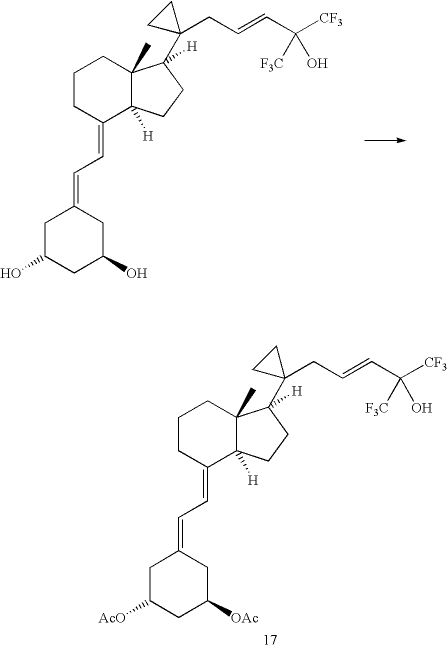

0.0328 g of 1,25-dihydroxy-20-cyclopropyl-23E-ene-26,27-hexafluoro-19-nor-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 5 mL of water and 20 mL of ethyl acetate. (Extraction of the aqueous layer gave no phosphomolybdic acid-detectable material). The organic layer was washed with 3×5 mL of water, once with 5 mL of saturated sodium hydrogen carbonate, once with 3 mL of brine then dried (sodium sulfate) and evaporated, the residue shows Rf 0.25 as the only spot. The oily residue was taken up in 1:6 ethyl acetate-hexane then flash-chromato-graphed on a 13.5×110 mm column using 1:6 ethyl acetate-hexane as mobile phase for fractions 1-10. Fractions 4-9 were pooled and evaporated, the residue taken up in methyl formate, filtered, then evaporated to give 0.0316 g of the title compound (17).

### Example 15

### Synthesis of 1,3-Di-O-acetyl-1,25-dihydroxy-20-cyclopropyl-23Z-ene-26,27-hexafluoro-19-nor-cholecalciferol (18)

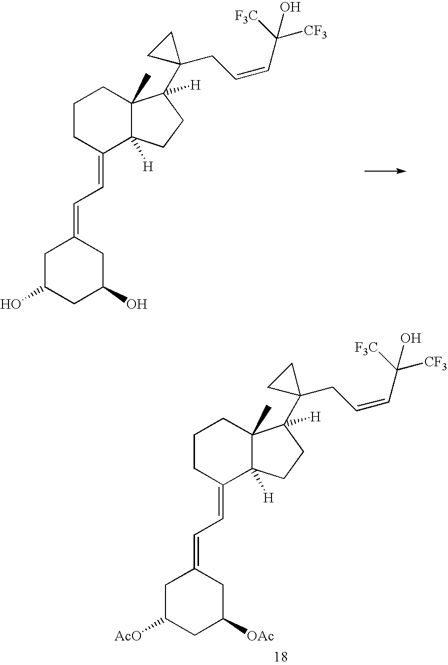

0.0429 g of 1,25-dihydroxy-20-cyclopropyl-23Z-ene-26,27-hexafluoro-19-nor-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 7 mL of water and 25 mL of ethyl acetate. The organic layer was washed with 3×5 mL of water, once with 5 mL of saturated sodium hydrogen carbonate, once with 3 mL of brine then dried (sodium sulfate, TLC (1:4 ethyl acetate-hexane shows mostly one spot) and evaporated, flash-chromatographed on a 15×120 mm column using 1:6 as mobile phase. Fractions 3-6 (20 mL each) were pooled and evaporated. The residue was taken up in methyl formate, filtered and evaporated, to give 0.0411 g of the title compound (18).

### Example 16

### Synthesis of 1,3-Di-O-acetyl-1,25-dihydroxy-20-cyclopropyl-cholecalciferol (19)

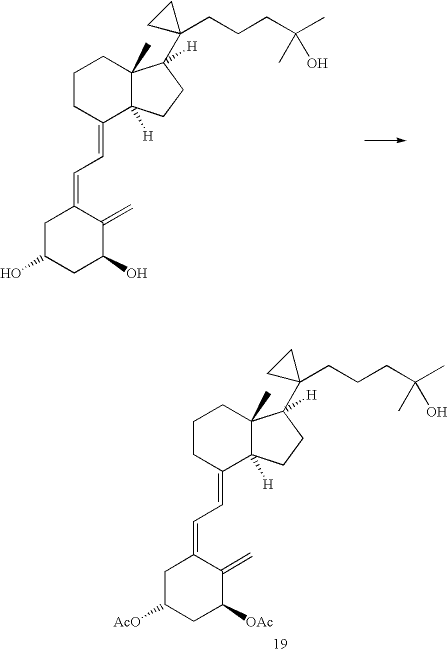

0.0797 g of 1,25-dihydroxy-20-cyclopropyl-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 10 mL of water and 25 mL of ethyl acetate. The organic layer was washed with 3×10 mL of water, once with 5 mL of saturated sodium hydrogen carbonate, once with 3 mL of brine then dried and evaporated, to give 0.1061 g of a tan oily residue that was flash-chromatographed on a 15×120 mm column using 1:6 as mobile phase. Fractions 9-16 (20 mL each) were pooled (TLC 1:4 ethyl acetate-hexane, Rf 0.13) and evaporated. This residue was taken up in methyl formate, filtered and evaporated to give 0.0581 g of the title compound (19).

### Example 17

### Synthesis of 1,3-Di-O-acetyl-1α,25-dihydroxy-16-ene-20-cyclopropyl-19-nor-cholecalciferol (20)

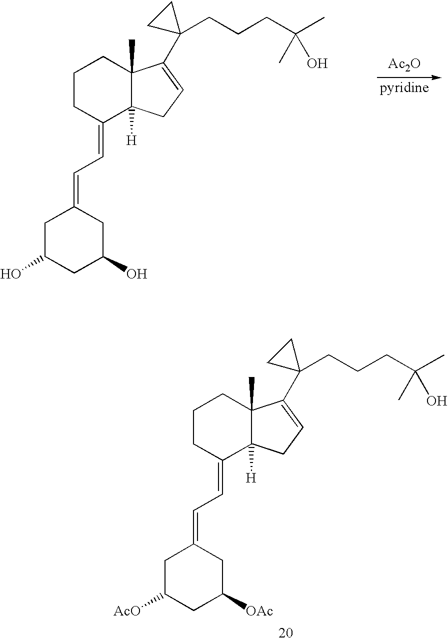

To the solution of 1α,25-Dihydroxy-16-ene-20-cyclopropyl-19-nor-cholecalciferol (94 mg, 0.23 mmol) in pyridine (3 mL) at 0° C., acetic anhydride (0.5 mL, 5.3 mmol) was added. The mixture was stirred for 1 h, refrigerated for 15 h. and then was stirred for additional 8 h. Water (10 mL) was added and after stirring for 15 min. the reaction mixture was extracted with AcOEt:Hexane 1:1 (25 mL), washed with water (4×25 mL) and brine (20 mL), dried over Na₂SO₄. The residue (120 mg) after evaporation of the solvent was purified by FC (15 g, 30% AcOEt in hexane) to give the titled compound (20) (91 mg, 0.18 mmol, 80%).
[α]³⁰_{D}=+14.4 c 0.34, EtOH

UV λmax (EtOH): 242 nm (ε34349.250 nm (ε 40458), 260 nm (ε 27545);

¹H NMR (CDCl₃): 6.25 (1H, d, J=11.1 Hz), 5.83 (1H, d, J=11.3 Hz), 5.35 (1H, m), 5.09 (2H, m), 2.82-1.98 (7H, m), 2.03 (3H, s), 1.98 (3H, s), 2.00-1.12 (15H, m), 1.18 (6H, s), 0.77 (3H, s), 0.80-0.36 (4H, m);

¹³C NMR (CDCl₃): 170.73(0), 170.65(0), 157.27(0), 142.55(0), 130.01(0), 125.06(1), 123.84(1), 115.71(1), 71.32(0), 70.24(1), 69.99(1), 59.68(1), 50.40(0), 44.08(2), 41.40(2), 38.37(2), 35.96(2), 35.80(2), 32.93(2), 29.48(3), 29.31(2), 28.71(2), 23.71(2), 22.50(2), 21.56(3), 21.51(0), 21.44(3), 18.01(3), 12.93(2), 10.53(2);

| | | | |
|---|---|---|---|
| MS HRES | Calculated for C₃₁H₄₆O₅ | M + Na | 521.3237 |
| | Observed | M + Na | 521.3233 |

### Example 18

### Synthesis of 1,3-Di-O-acetyl-1α,25-hydroxy-16-ene-20-cyclopropyl-cholecalciferol (21)

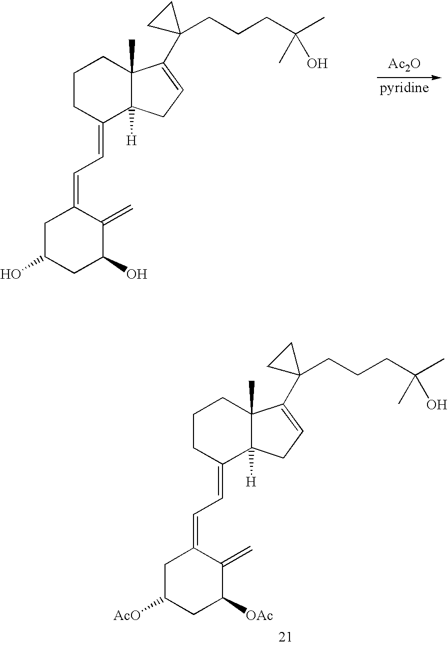

To the solution of 1α,25-Dihydroxy-16-ene-20-cyclopropyl-cholecalciferol (100 mg, 0.23 mmol) in pyridine (3 mL) at 0° C., acetic anhydride (0.5 mL, 5.3 mmol) was added. The mixture was stirred for 2 h and then refrigerated for additional 15 h. Water (10 mL) was added and after stirring for 15 min. the reaction mixture was extracted with AcOEt: Hexane 1:1 (25 mL), washed with water (4×25 mL), brine (20 mL) and dried over Na₂SO₄. The residue (150 mg) after evaporation of the solvent was purified by FC (15 g, 30% AcOEt in hexane) to give the titled compound (21) (92 mg, 0.18 mmol, 78%)
[α]³⁰_{D}=−14.9 c 0.37, EtOH

UV λmax (EtOH): 208 nm (ε 15949), 265 nm (ε 15745);

¹H NMR (CDCl₃): 6.34 (1H, d, J=11.3 Hz), 5.99 (1H, d, J=11.3 Hz), 5.47 (1H, m), 5.33 (1H, m), 5.31 (1H, s), 5.18 (1H, m), 5.04 (1H, s), 2.78 (1H, m), 2.64 (1H, m), 2.40-1.10 (18H, m), 2.05 (3H, s), 2.01 (3H, s), 1.18 (6H, s), 0.76 (3H, s), 0.66-0.24 (4H, m); ¹³C NMR (CDCl₃): 170.76(0), 170.22(0), 157.18(0), 143.02(0), 142.40(0), 131.94(0), 125.31(1), 125.10(1), 117.40(1), 115.22(2), 72.97(1), 71.32(0), 69.65(1), 59.71(1), 50.57(0), 44.07(2), 41.73(2), 38.36(2), 37.10(2), 35.80(2), 29.45(3), 29.35(2), 29.25(3), 28.92(2), 23.80(2), 22.48(2), 21.55(3), 21.50(3), 21.35(0), 17.90(3), 12.92(2), 10.54(2);

| | | | |
|---|---|---|---|
| MS HRES | Calculated for C₃₂H₄₆O₅ | M + Na | 533.3237 |
| | Observed | M + Na | 533.3236 |

### Example 19

### Synthesis of 1,3-Di-O-acetyl-1,25-dihydroxy-23-yne-cholecalciferol (22)

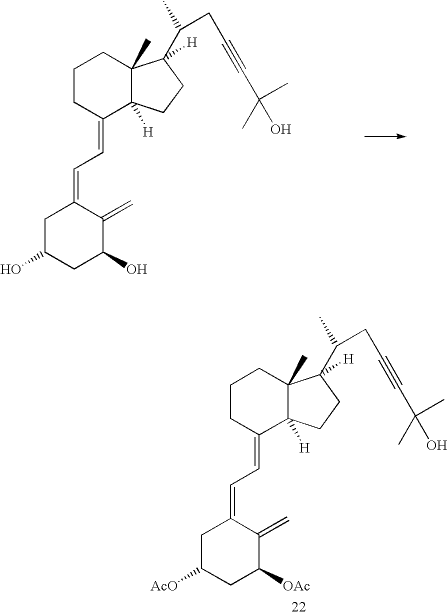

0.2007 g of (0.486 mmol) was dissolved in 2 mL of pyridine. This solution was cooled in an ice bath and 0.6 mL of acetic anhydride was added. The solution was kept in an ice bath for 45 h then diluted with 10 mL of water, stirred for 10 min and equilibrated with mL of water and 40 mL of ethyl acetate. The organic layer was washed with 4×20 mL of water, 10 mL of brine, dried (sodium sulfate) and evaporated. The brown, oily residue was flash chromatographed using 1:19, 1:9, and 1:4 ethyl acetate-hexane as stepwise gradient. The main band with Rf 0.16 (TLC 1:4 acetate-hexane) was evaporated to give 1,3-di-O-acetyl-1,25-dihydroxy-23-yne-cholecalciferol (22) a colorless foam, 0.0939 g.

### Example 20

### Synthesis of (3aR,4S,7aR)-7a-Methyl-1-[1-(4-hydroxy-4-methyl-pent-2-ynyl)-cyclopropyl]-3a,4,5,6,7,7a-hexahydro-3H-inden-4-ol

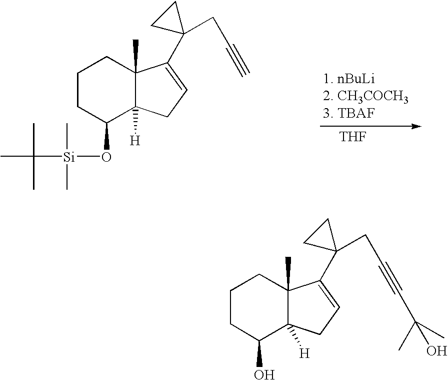

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])-cyclopropyl}-ethynyl (1.0 g, 2.90 mmol) in tetrahydrofurane (15 mL) at −78° C. was added n-BuLi (2.72 mL, 4.35 mmol, 1.6M in hexane). After stirring at −78° C. for 1 h., acetone (2.5 mL, 34.6 mmol) was added and the stirring was continued for 2.5 h. NH₄Cl_{aq} was added (15 mL) and the mixture was stirred for 15 min at room temperature then extracted with AcOEt (2×50 mL). The combined extracts were washed with brine (50 mL) and dried over Na₂SO₄. The residue after evaporation of the solvent (2.4 g) was purified by FC (50 g, 10% AcOEt in hexane) to give (3aR,4S,7aR)-5-(1-[4-(tert-Butyl-dimethyl-silanyloxy)-7a-methyl-3a,4,5,6,7,7a-hexahydro-3H-inden-1-yl]-cyclopropyl)-2-methyl-pent-3-yn-2-ol (1.05 g, 2.61 mmol) which was treated with tetrabutylammonium fluoride (6 mL, 6 mmol, 1.0M in THF) and stirred at 65-75° C. for 48 h. The mixture was diluted with AcOEt (25 mL) and washed with water (5×25 mL), brine (25 mL). The combined aqueous washes were extracted with AcOEt (25 mL) and the combined organic extracts were dried over Na₂SO₄. The residue after evaporation of the solvent (1.1 g) was purified by FC (50 g, 20% AcOEt in hexane) to give the titled compound (0.75 g, 2.59 mmol, 90%). [α]³⁰_{D}=+2.7 c 0.75, CHCl₃. ¹H NMR (CDCl₃): 5.50 (1H, m), 4.18 (1H, m), 2.40 (2H, s), 2.35-1.16 (11H, m), 1.48 (6H, s), 1.20 (3H, s), 0.76-0.50 (4H, m); ¹³C NMR (CDCl₃): 156.39, 125.26, 86.39, 80.19, 69.21, 65.16, 55.14, 46.94, 35.79, 33.60, 31.67, 29.91, 27.22, 19.32, 19.19, 17.73, 10.94, 10.37;

MS HREI Calculated for C₂₂H₂₈O₂ M+ 288.2089 Observed M+ 288.2091.

### Example 21

### Synthesis of (3aR,4S,7aR)-7a-Methyl-1-[1-(4-hydroxy-4-methyl-pent-2Z-enyl)-cyclopropyl]-3a,4,5,6,7,7a-hexahydro-3H-inden-4-ol

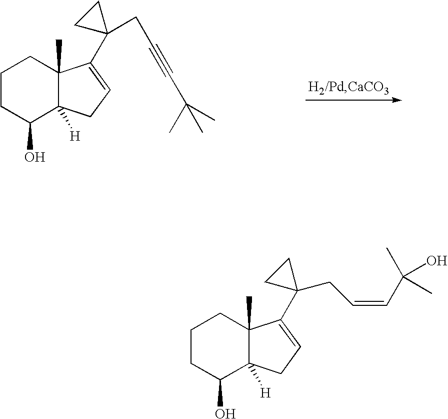

The mixture of (3aR,4S,7aR)-7a-Methyl-1-[1-(−4-hydroxy-4-methyl-pent-2-ynyl)-cyclopropyl]-3a,4,5,6,7,7a-hexahydro-3H-inden-4-ol (0.72 g, 2.50 mmol), ethyl acetate (10 mL), hexane (24 mL), absolute ethanol (0.9 mL), quinoline (47 □L) and Lindlar catalyst (156 mg, 5% Pd on CaCO₃) was hydrogenated at room temperature for 2 h. The reaction mixture was filtered through a celite pad and the pad was washed with AcOEt. The filtrates and the washes were combined and washed with 1M HCl, NaHCO₃ and brine. After drying over Na₂SO₄ the solvent was evaporated and the residue (0.79 g) was purified by FC (45 g, 20% AcOEt in hexane) to give the titled compound (640 mg, 2.2 mmol, 88%).

### Example 22

### Synthesis of (3aR,4S,7aR)-7a-Methyl-1-[1-(4-hydroxy-4-methyl-pentyl)-cyclopropyl]-3a,4,5,6,7,7a-hexahydro-3H-inden-4-ol

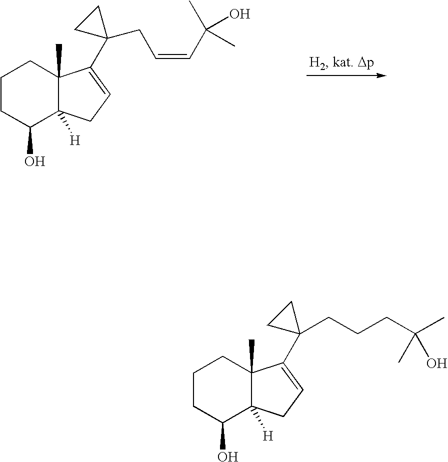

The mixture of (3aR,4S,7aR)-7a-Methyl-1-[1-(4-hydroxy-4-methyl-pent-2Z-enyl)-cyclopropyl]-3a,4,5,6,7,7a-hexahydro-3H-inden-4-ol (100 mg, 0.34 mmol), 1,4-bis(diphenyl-phosphino)butane 1,5 cyclooctadiene rhodium tetrafluoroborate (25 mg, 0.034 mmol), dichloromethane (5 mL) and one drop of mercury was hydrogenated using Paar apparatus at room temperature and 50 p.s.i. pressure for 3 h. The reaction mixture was filtered through Celite pad, which was then washed with ethyl acetate. The combine filtrates and washes were evaporated to dryness (110 mg) and purified by FC (10 g, 20% AcOEt in hexane) to give the titled compound (75 mg, 0.26 mmol, 75%). [α]³⁰_{D}=−8.5 c 0.65, CHCl₃. 1H NMR (CDCl₃): 5.37 (1H, m), 4.14 (1H, m), 2.37-1.16 (17H, m), 1.19 (6H, s), 1.18 (31H, s), 0.66-0.24 (4H, m);

MS HREI Calculated for C₁₉H₃₂O₂ M+H 292.2402. Observed M+H 292.2404.

### Example 23

### Synthesis of (3aR,7aR)-7a-Methyl-1-[1-(4-methyl-4-trimethylsilanyloxy-pentyl)-cyclopropyl]-3a,4,5,6,7,7a-hexahydro-3H-inden-4-one

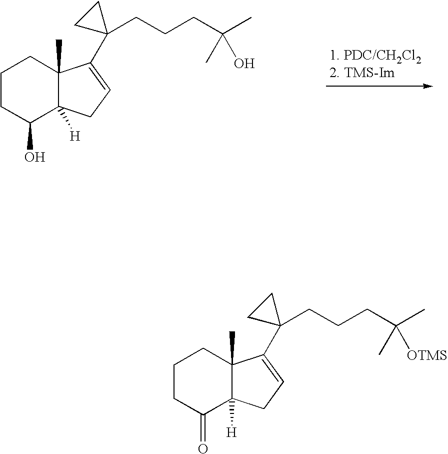

To a stirred suspension of (3aR,4S,7aR)-7a-Methyl-1-[1-(4-hydroxy-4-methyl-pentenyl)-cyclopropyl]-3a,4,5,6,7,7a-hexahydro-3H-inden-4-ol (440 mg, 1.50 mmol) and Celite (2.0 g) in dichloromethane (10 mL) at room temperature wad added pyridinium dichromate (1.13 g, 3.0 mmol). The resulting mixture was stirred for 5 h filtered through silica gel (10 g), and then silica gel pad was washed with 20% AcOEt in hexane. The combined filtrate and washes were evaporated, to give a crude (3aR,7aR)-7a-Methyl-1-[1-(4-hydroxy-4-methyl-pentenyl)-cyclopropyl]-3a,4,5,6,7,7a-hexahydro-3H-inden-4-one (426 mg, 1.47 mmol, 98%). To a stirred solution of (3aR,7aR)-7a-Methyl-1-[1-(4-hydroxy-4-methyl-pentenyl)-cyclopropyl]-3a,4,5,6,7,7a-hexahydro-3H-inden-4-one (424 mg, 1.47 mmol) in dichloromethane (10 mL) at room temperature was added trimethylsilyl-imidazole (0.44 mL, 3.0 mmol). The resulting mixture was stirred for 1.0 h filtered through silica gel (10 g) and the silica gel pad was washed with 10% AcOEt in hexane. Combined filtered and washes were evaporated to give the titled compound (460 mg, 1.27 mmol, 86%). [α]²⁹_{D}=−9.9 c 0.55, CHCl₃. ¹H NMR (CDCl₃): 5.33 (1H, dd, J=3.2, 1.5 Hz), 2.81 (1H, dd, J=10.7, 6.2 Hz), 2.44 (1H, ddd, J=15.6, 10.7, 1.5 Hz), 2.30-1.15 (13H, m) overlapping 2.03 (ddd, J=15.8, 6.4, 3.2 Hz), 1.18 (6H, s), 0.92 (3H, s), 0.66-0.28 (4H, m), 0.08 (9H, s); ¹³C NMR (CDCl₃): 211.08 (0), 155.32(0), 124.77(1), 73.98(0), 64.32(1), 53.91(0), 44.70(2), 40.45(2), 38.12(2), 34.70(2), 29.86(3), 29.80(3), 26.80(2), 24.07(2), 22.28(2), 21.24(0), 18.35(3), 12.60(2), 10.64(2), 2.63 (3);

MS HRES Calculated for C₂₂H₃₈O₂Si M+ 362.2641. Observed M+ 362.2648.

### Example 24

### Synthesis of (3aR,7aR)-7a-Methyl-1-[1-(4-methyl-4-trimethylsilanyloxy-pent-2-ynyl)-cyclopropyl]-3a,4,5,6,7,7a-hexahydro-3H-inden-4-one

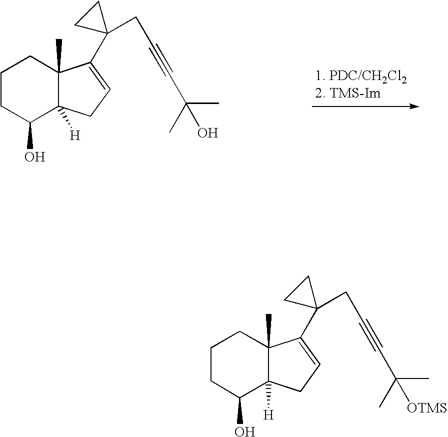

To a stirred suspension of (3aR,4S,7aR)-7a-Methyl-1-[1-(4-hydroxy-4-methyl-pent-2-ynyll)-cyclopropyl]-3a,4,5,6,7,7a-hexahydro-3H-inden-4-ol (381 mg, 1.32 mmol) and Celite (2.0 g) in dichloromethane (10 mL) at room temperature wad added pyridinium dichromate (1.0 g, 2.65 mmol). The resulting mixture was stirred for 1.5 h filtered through silica gel (10 g), and then silica gel pad was washed with 20% AcOEt in hexane. The combined filtrate and washes were evaporated, to give a crude (3aR,7aR)-7a-Methyl-1-[1-(4-hydroxy-4-methyl-pent-2-ynyll)-cyclopropyl]-3a,4,5,6,7,7a-hexahydro-3H-inden-4-one (360 mg, 1.26 mmol, 95%). To a stirred solution of (3aR,7aR)-7a-Methyl-1-[1-(4-hydroxy-4-methyl-pent-2-ynyll)-cyclopropyl]-3a,4,5,6,7,7a-hexahydro-3H-inden-4-one (360 mg, 1.26 mmol) in dichloromethane (10 mL) at room temperature was added trimethylsilyl-imidazole (0.25 mL, 1.7 mmol). The resulting mixture was stirred for 0.5 h filtered through silica gel (10 g) and the silica gel pad was washed with 5% AcOEt in hexane. Combined filtered and washes were evaporated to give the titled compound (382 mg, 1.07 mmol, 81%).

### Example 25

### Synthesis of 1α,25-Dihydroxy-16-ene-20-cyclopropyl-23,24-yne-cholecalciferol (23)

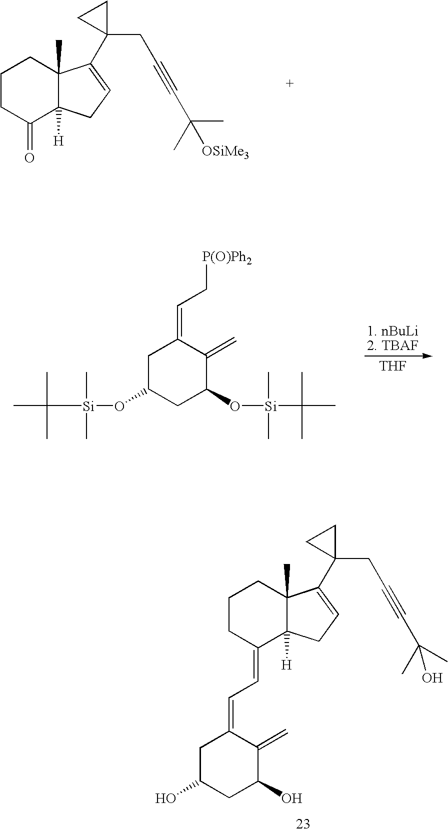

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (513 mg, 0.88 mmol) in tetrahydrofurane (6 mL) at −78° C. was added n-BuLi (0.55 mL, 0.88 mmol). The resulting mixture was stirred for 15 min and solution of (3aR,7aR)-7a-Methyl-1-[1-(4-methyl-4-trimethylsilanyloxy-pent-2-ynyll)-cyclopropyl]-3a,4,5,6,7,7a-hexahydro-3H-inden-4-one (179 mg, 0.50 mmol, in tetrahydrofurane (2 mL) was added dropwise. The reaction mixture was stirred at −72° C. for 3.5 h diluted with hexane (25 mL) washed brine (30 mL) and dried over Na₂SO₄. The residue (716 mg) after evaporation of the solvent was purified by FC (15 g, 5% AcOEt in hexane) to give 1α,3β-Di(tert-Butyl-dimethyl-silanyloxy)-25-trimethylsilanyloxy-16-ene-20-cyclopropyl-23,24-yne-cholecalciferol (324 mg, 045 mmol). To the 1α,3β-Di(tert-Butyl-dimethyl-silanyloxy)-25-trimethylsilanyloxy-16-ene-20-cyclopropyl-23,24-yne-cholecalciferol (322 mg, 0.45 mmol) tetrabutylammonium fluoride (4 mL, 4 mmol, 1M solution in THF) was added, at room temperature. The mixture was stirred for 18 h. diluted with AcOEt (25 mL) and washed with water (5×20 mL), brine (20 mL) and dried over Na₂SO₄. The residue (280 mg) after evaporation of the solvent was purified by FC (10 g, 50% AcOEt in hexane and AcOEt) to give the titled compound (23) (172 mg, 0.41 mmol, 82%). [α]31=+32.4 c 0.50, MeOH. UV λmax (EtOH): 261 nm (ε 11930); ¹H NMR (CDCl₃): 6.36 (1H, d, J=11.3 Hz), 6.09 (1H, d, J=11.3 Hz), 5.45(1H, m), 5.33 (1H, m), 5.01 (1H, s), 4.45 (1H, m), 4.22 (1H, m), 2.80 (1H, m), 2.60 (1H, m), 2.50-1.10 (16H, m), 1.45 (6H, s), 0.81 (3H, s), 0.72-0.50 (4H, m); MS HRES Calculated for C₂₉H₃₈O₃ M+ 422.2821. Observed M+ 422.2854.

### Example 26

### Synthesis of 1α,25-Dihydroxy-16-ene-20-cyclopropyl-23,24-yne-19-nor-cholecalciferol (24)

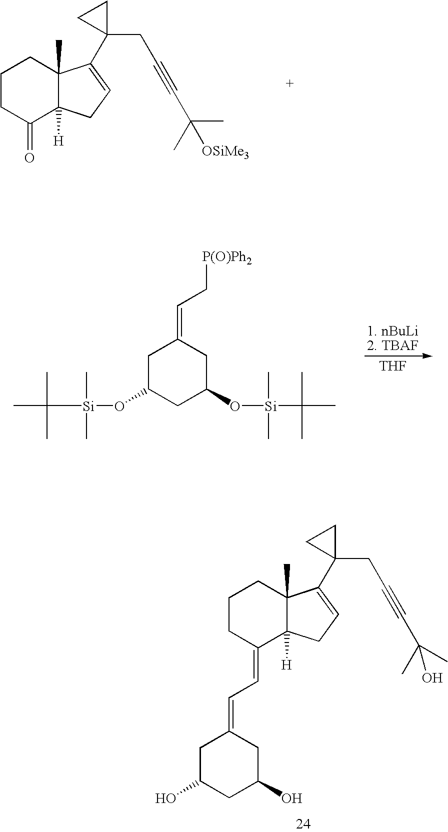

To a stirred solution of a (1R,3R)-1,3-bis-((tert-butyldimethyl)silanyloxy)-5-[2-(diphenylphosphinoyl)ethylidene]-cyclohexane (674 mg, 1.18 mmol) in tetrahydrofurane (8 mL) at −78° C. was added n-BuLi (0.74 mL, 1.18 mmol). The resulting mixture was stirred for 15 min and solution of (3aR,7aR)-7a-Methyl-1-[1-(4-methyl-4-trimethylsilanyloxy-pent-2-ynyl)-cyclopropyl]-3a,4,5,6,7,7a-hexahydro-3H-inden-4-one (235 mg, 0.66 mmol, in tetrahydrofurane (3 mL) was added dropwise. The reaction mixture was stirred at −72° C. for 3.5 h diluted with hexane (25 mL) washed brine (30 mL) and dried over Na₂SO₄. The residue (850 mg) after evaporation of the solvent was purified by FC (15 g, 5% AcOEt in hexane) to give 1α,3β-Di(tert-Butyl-dimethyl-silanyloxy)-25-trimethylsilanyloxy-16-ene-20-cyclopropyl-23,24-yne-19-nor-cholecalciferol (330 mg, 0.46 mmol). To the 1α,3β-Di(tert-Butyl-dimethyl-silanyloxy)-25-trimethylsilanyloxy-16-ene-20-cyclopropyl-23,24-yne-19-nor-cholecalciferol (328 mg, 0.46 mmol) tetrabutylammonium fluoride (5 mL, 5 mmol, 1M solution in THF) was added, at room temperature. The mixture was stirred for 62 h. diluted with AcOEt (25 mL) and washed with water (5×20 mL), brine (20 mL) and dried over Na₂SO₄. The residue (410 mg) after evaporation of the solvent was purified by FC (10 g, 50% AcOEt in hexane and AcOEt) to give the titled compound (24) (183 mg, 0.45 mmol, 68%). [α]²⁹_{D}=+72.1 c 0.58, MeOH. UV % max (EtOH): 242 nm (ε29286), 251 nm (ε 34518), 260 nm (ε 23875); ¹H NMR (CDCl₃): 6.30 (1H, d, J=11.3 Hz), 5.94 (1H, d, J=11.3 Hz), 5.48 (1H, m), 4.14 (1H, m), 4.07 (1H, m), 2.78 (2H, m), 2.52-1.10 (18H, m), 1.49(6H, s), 0.81 (3H, s), 0.72-0.50 (4H, m); MS HRES Calculated for C₂₇H₃₈O₃ M+ 410.2821. Observed M+ 410.2823.

### Example 27

### Synthesis of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol

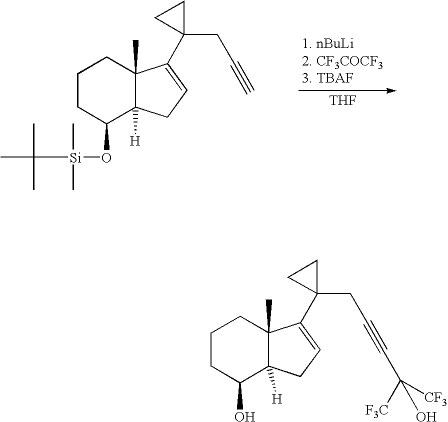

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])-cyclopropyl}-ethynyl (1.95 g, 5.66 mmol) in tetrahydrofurane (35 mL) at −78° C. was added n-BuLi (4.3 mL, 6.88 mmol, 1.6M in hexane). After stirring at −78° C. for 1 h., hexafluoroacetone (six drops from the cooling finger) was added and the stirring was continued for 1 h. NH₄Cl_{aq} was added (10 mL) and the mixture was allowed to warm to room temperature. The reaction mixture was diluted with brine (100 mL) and extracted with hexane (2×125 mL). The combined extracts were dried over Na₂SO₄. The residue after evaporation of the solvent (8.2 g) was purified by FC (150 g, 10% AcOEt in hexane) to give (3aR,4S,7aR)-5-{1-[4-(tert-Butyl-dimethyl-silanyloxy)-7a-methyl-3a,4,5,6,7,7a-hexahydro-3H-inden-1-yl]-cyclopropyl}-1,1,1-trifluoro-2-trifluoromethyl-pent-3-yn-2-ol (2.73 g, 5.35 mmol) which was treated with tetrabutylammonium fluoride (20 mL, 20 mmol, 1.0M in THF) and stirred at 65-75° C. for 30 h. The mixture was diluted with AcOEt (150 mL) and washed with water (5×150 mL), brine (150 mL). The combined aqueous washes were extracted with AcOEt (150 mL) and the combined organic extracts were dried over Na₂SO₄. The residue after evaporation of the solvent (3.2 g) was purified by FC (150 g, 20% AcOEt in hexane) to give the titled compound (2.05 g, 5.17 mmol, 97%). [α]²⁸_{D}=+6.0 c 0.47, CHCl₃. ¹H NMR (CDCl₃): 5.50 (1H, br. s), 4.16 (1H, br. s), 3.91 (1H, s), 2.48 (1H, part A of the AB quartet, J=17.5 Hz), 2.43 (1H, part B of the AB quartet, J=17.5 Hz), 2.27 (1H, m), 2.00-1.40 (9H, m), 1.18 (3H, s), 0.8-0.5 (4H, m); ¹³C NMR (CDCl₃): 155.26(0), 126.68(1), 121.32(0, q, J=284 Hz), 90.24(0), 71.44(0, sep. J=34 Hz), 70.54 (0), 69.57(1), 55.17(1), 47.17(0), 36.05(2), 33.63(2), 30.10(2), 27.94(2), 19.50(3), 19.27(0), 17.90(2), 11.56(2), 11.21(2); MS HREI Calculated for C₁₉H₂₂O₂F₆ M+ 396.1524. Observed M+ 396.1513.

### Example 28

### Synthesis of (3aR,7aR-7a-Methyl-1-[1-(5,5,5-trifuoro-4-trifluoromethyl-4-hydroxy-pen-2-ynyl)-cyclopropyl]-3a,4,5,6,7,7a-hexahydro-3H-inden-4-one

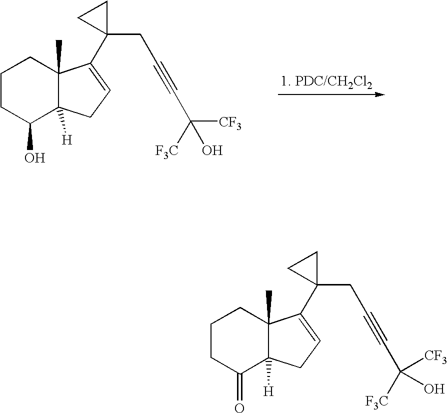

To a stirred suspension of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol (504 mg, 1.27 mmol) and Celite (1.5 g) in dichloromethane (12 mL) at room temperature wad added pyridinium dichromate (0.98 g, 2.6 mmol). The resulting mixture was stirred for 2.5 h filtered through silica gel (5 g), and then silica gel pad was washed with 20% AcOEt in hexane. The combined filtrate and washes were evaporated, to give a titled compound (424 mg, 1.08 mmol, 85%). [α]²⁸_{D}=+3.1 c 0.55, CHCl₃. ¹H NMR (CDCl₃): 5.46 (1H, br. s), 3.537 (1H, s), 2.81 (1H, dd, J=10.7, 6.5 Hz), 2.49-1.76 (10H, m), 0.90 (3H, s), 0.77-0.53 (4H, m); MS HREI Calculated for C₁₉H₂₀O₂F₆ M+H 395.1440. Observed M+H 395.1443.

### Example 29

### Synthesis of 1α,25-Dihydroxy-16-ene-20-cyclopropyl-23,24-yne-26,27-hexafluoro 19-nor-cholecalciferol (25)

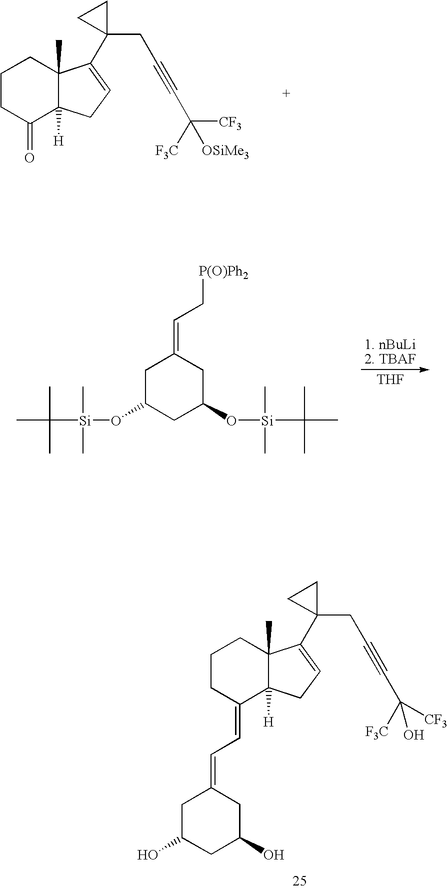

To a stirred solution of a (1R,3R)-1,3-bis-((tert-butyldimethyl)silanyloxy)-5-[2-(diphenylphosphinoyl)ethylidene]-cyclohexane (900 mg, 1.58 mmol) in tetrahydrofurane (8 mL) at −78° C. was added n-BuLi (1.0 mL, 1.6 mmol). The resulting mixture was stirred for 15 min and solution of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-3H-inden-4-one (200 mg, 0.51 mmol, in tetrahydrofurane (3 mL) was added dropwise. The reaction mixture was stirred at −72° C. for 3.5 h diluted with hexane (25 mL) washed brine (30 mL) and dried over Na₂SO₄. The residue (850 mg) after evaporation of the solvent was purified by FC (20 g, 10% AcOEt in hexane) to give 1α,3β-Di(tert-Butyl-dimethyl-silanyloxy)-25-hydroxy-16-ene-20-cyclopropyl-23,24-yne-26,27-hexafluoro-19-nor-cholecalciferol (327 mg, 0.44 mmol, 86%). To the 1α,3β-Di(tert-Butyl-dimethyl-silanyloxy)-25-hydroxy-16-ene-20-cyclopropyl-23,24-yne-26,27-hexafluoro-19-nor-cholecalciferol (327 mg, 0.44 mmol). Tetrabutylammonium fluoride (4 mL, 4 mmol, 1M solution in THF) was added, at room temperature. The mixture was stirred for 24 h. diluted with AcOEt (25 mL) and washed with water (5×20 mL), brine (20 mL) and dried over Na₂SO₄. The residue (250 mg) after evaporation of the solvent was purified by FC (10 g, 50% AcOEt in hexane and AcOEt) to give the titled compound (25) (183 mg, 0.45 mmol, 68%). [α]³⁰_{D}=+73.3 c 0.51, EtOH. UV λmax (EtOH): 243 nm (629384251 nm (E 34973), 260 nm (E 23924); ¹H NMR (CDCl₃): 6.29 (1H, d, J=11.1 Hz), 5.93 (1H, d, J=11.1 Hz), 5.50 (1H, m), 4.12 (1H, m), 4.05 (1H, m), 2.76 (2H, m), 2.55-1.52 (18H, m), 0.80 (3H, s), 0.80-0.49 (4H, m); ¹³C NMR (CDCl₃): 155.24(0), 141.78(0), 131.28(0), 126.23(1), 123.65(1), 121.09(0, q, J=285 Hz), 115.67(1), 89.63(0), 70.42(0), 67.48(1), 67.29(1), 59.19(1), 49.87(0), 44.49(2), 41.98(2), 37.14(2), 35.76(2), 29.22(2), 28.47(2), 27.57(2), 23.46(2), 19.32(0), 17.97(3), 11.89(2), 10.18(2); MS HRES Calculated for C₂₇H₃₂O₃F₆ M+H 519.2329. Observed M+H 519.2325.

### Example 30

### Synthesis of 1α,25-Dihydroxy-16-ene-20-cyclopropyl-23,24-yne-26,27 hexafluoro cholecalciferol (26)

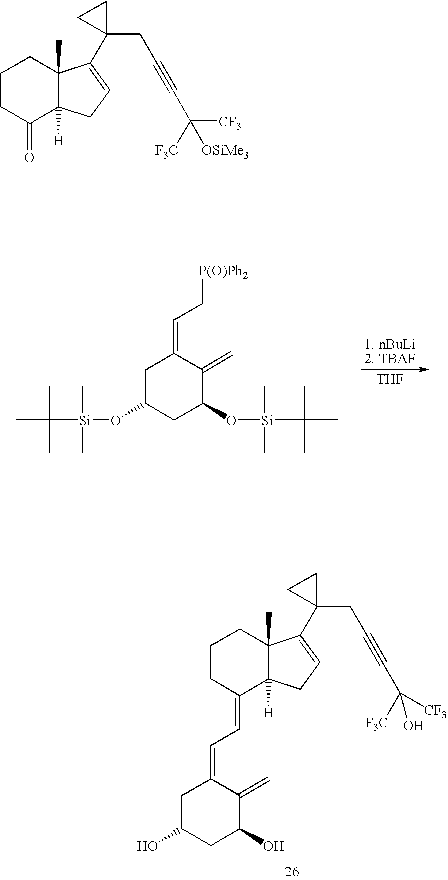

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (921 mg, 1.58 mmol) in tetrahydrofurane (8 mL) at −78° C. was added n-BuLi (1.0 mL, 1.6 mmol). The resulting mixture was stirred for 15 min and solution of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-3H-inden-4-one (197 mg, 0.50 mmol, in tetrahydrofurane (2 mL) was added dropwise. The reaction mixture was stirred at −72° C. for 3.5 h diluted with hexane (25 mL) washed brine (30 mL) and dried over Na₂SO₄. The residue (876 mg) after evaporation of the solvent was purified by FC (20 g, 105% AcOEt in hexane) to give 1α,3β-Di(tert-Butyl-dimethyl-silanyloxy)-25-hydroxy-16-ene-20-cyclopropyl-23,24-yne-26,27-hexafluoro-cholecalciferol (356 mg, 0.47 mmol). To the 1α,3β-Di(tert-Butyl-dimethyl-silanyloxy)-25-hydroxy-16-ene-20-cyclopropyl-23,24-yne-26,27-hexafluoro-cholecalciferol (356 mg, 0.47 mmol) tetrabutylammonium fluoride (5 mL, 5 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (270 mg) after evaporation of the solvent was purified by FC (20 g, 50% AcOEt in hexane and AcOEt) to give the titled compound (26) (216 mg, 0.41 mmol, 87%). [α]³⁰_{D}=+40.0 c 0.53, EtOH. UV λmax (EtOH): 262 nm (c 12919); ¹H NMR (CDCl₃): 6.38 (1H, d, J=11.5 Hz), 6.10 (1H, d, J=11.1 Hz), 5.49 (1H, m), 5.35 (1H, s), 5.02 (1H, s), 4.45 (1H, m), 4.25 (1H, m), 3.57 (1H, s), 2.83-1.45 (18H, m), 0.82 (3H, s), 0.80-0.51 (4H, m); MS HRES Calculated for C₂₈H₃₂O₃F₆ M+H 531.2329. Observed M+H 531.2337.

### Example 31

### Synthesis of (3aR,4S,7aR)-7a-Methyl-1-[1-(5,5,5-trifluoro-4-hydroxy-4-trifluoromethyl-pent-2 E-enyl)-cyclopropyl]-3a,4,5,6,7,7a-hexahydro-3H-inden-4-ol

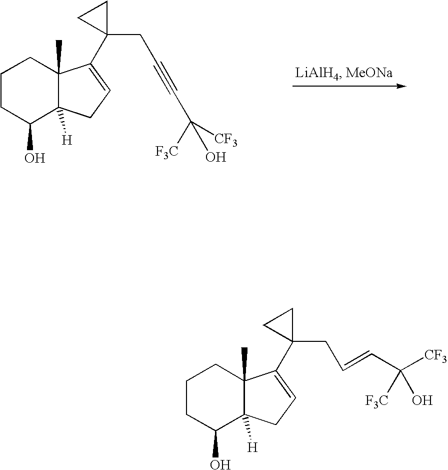

To a lithium aluminum hydride (4.5 mL, 4.5 mmol, 1.0M in THF) at 5° C. was added first solid sodium methoxide (245 mg, 4.6 mmol) and then dropwise solution of (3aR, 4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol (360 mg, 0.91 mmol) in tetrahydrofurane (5 mL). After addition was completed the mixture was stirred under reflux for 2.5 h. Tehn it was cooled in the ice-bath and quenched with water (2.0 mL) and sodium hydroxide (2.0 mL, 2.0 M water solution); diluted with ether (50 mL) stirred for 30 min, MgSO₄ (5 g) was than added and stirring was continued for 30 min. The residue after evaporation of the filtrates (0.42 g) was purified by FC (20 g, 20% AcOEt in hexane) to give the titled compound (315 mg, 0.79 mmol, 87%). [α]²⁸_{D}=+2.0 c 0.41, CHCl₃. ¹H NMR (CDCl₃): 6.24 (1H, dt, J=15.7, 6.7 Hz), 5.60 (1H, d, J=15.7 Hz), 5.38 (1H, br. s), 4.13 (1H, br. s), 3.27 (1H, s), 2.32-1.34 (12H, m), 1.15 (3H, s), 0.80-0.45 (4H, m); ¹³C NMR (CDCl₃): 155.89(0), 138.10(1), 126.21(1), 122.50(0, q, J=287 Hz), 119.15 (1), 76.09(0, sep. J=31 Hz), 69.57(1), 55.33(1), 47.30(0), 40.31(2), 36.05(2), 33.71(2), 30.10(2), 20.36(0), 19.46(3), 17.94(2), 11.96(2), 11.46(2); MS REI Calculated for C₁₉H₂₄O₂F₆ M+ 398.1680. Observed M+ 398.1675.

### Example 32

### Synthesis of (3aR,7aR-7a-Methyl-1-[1-(5,5,5-trifluoro-4-trifluoromethyl-4-trimethylsilanyloxy-pen-2E-enyl)-cyclopropyl]-3a,4,5,6,7,7a-hexahydro-3H-inden-4-one

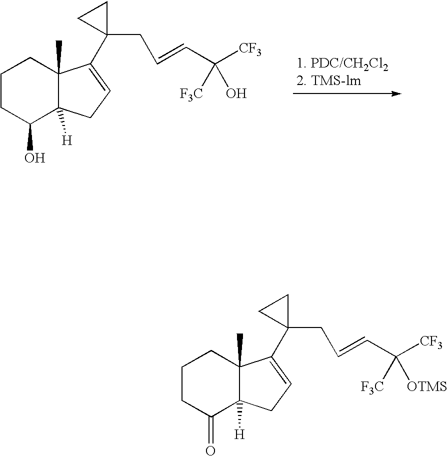

To a stirred suspension of (3aR,4S,7aR)-7a-Methyl-1-[1-(5,5,5-trifluoro-4-hydroxy-4-trifluoromethyl-pent-2E-enyl)-cyclopropyl-3a,4,5,6,7,7a-hexahydro-3H-inden-4-ol (600 mg, 1.51 mmol) and Celite (2.0 g) in dichloromethane (10 mL) at room temperature wad added pyridinium dichromate (1.13 g, 3.0 mmol). The resulting mixture was stirred for 3.5 h filtered through silica gel (10 g), and then silica gel pad was washed with 25% AcOEt in hexane; The combined filtrate and washes were evaporated, to give a crude (3aR,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-one (550 mg, 1.39 mmol, 92%). To a stirred solution of (3aR,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-one (550 mg, 1.39 mmol) in dichloromethane (15 mL) at room temperature was added trimethylsilyl-imidazole (1.76 mL, 12.0 mmol). The resulting mixture was stirred for 1.0 h filtered through silica gel (10 g) and the silica gel pad was washed with 10% AcOEt in hexane. Combined filtered and washes were evaporated to give the titled compound (623 mg, 1.33 mmol, 88%). [α]²⁸_{D}=−1.6 c 0.51, CHCl₃. ¹H NMR (CDCl₃): 6.14 (1H, dt, J=15.5, 6.7 Hz), 5.55 (1H, d, J=15.5 Hz), 5.35 (1H, m), 2.80 (1H, dd, J=10.7, 6.4 Hz), 2.47-1.74 (10H, m), 0.90 (3H, s), 0.76-0.40 (4H, m), 0.2 (9H, s); ¹³C NMR (CDCl₃): 210.99 (0), 154.28(0), 137.41(1), 126.26(1), 122.59(0, q, J=289 Hz), 120.89 (1), 64.31(1), 53.96(0), 40.60(2), 40.13(2), 35.00(2), 27.03(2), 24.21(2), 20.57(0), 18.53(3), 12.41(2), 10.79(2), 1.65 (3); MS HRES Calculated for C₂₂H₃₀O₂F₆Si M+H 469.1992. Observed M+H 469.1995.

### Example 33

### Synthesis of 1α,25-Dihydroxy-16-ene-20-cyclopropyl-23,24-E-ene-26,27-hexafluoro 19-nor-cholecalciferol (27)

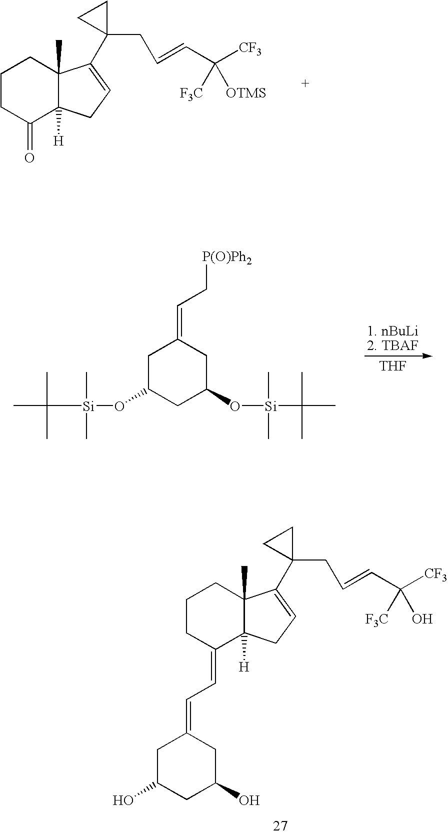

To a stirred solution of a (1R,3R)-1,3-bis-((tert-butyldimethyl)silanyloxy)-5-[2-(diphenylphosphinoyl)ethylidene]-cyclohexane (514 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[ ]-(5,5,5-trifluoro-4-trifluoromethyl-4-trimethylsilanyloxy-pent-2E-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (750 mg) after evaporation of the solvent was purified by FC (15 g, 5% AcOEt in hexane) to give a mixture of 1α,3β-Di(tert-Butyl-dimethyl-silanyloxy)-25-trimethylsilanyloxy-16-ene-20-cyclopropyl-23,24-E-ene-26,27-hexafluoro-19-nor-cholecalciferol and 1α,3β-Di(tert-Butyl-dimethyl-silanyloxy)₂₅-hydroxy-16-ene-20-cyclopropyl-23,24-E-ene-26,27-hexafluoro-19-nor-cholecalciferol (250 mg). To the mixture of 1α,3β-Di(tert-Butyl-dimethyl-silanyloxy)-25-trimethylsilanyloxy-16-ene-20-cyclopropyl-23,24-E-ene-26,27-hexafluoro-19-nor-cholecalciferol and 1α,3β-Di(tert-Butyl-dimethyl-silanyloxy)-25-hydroxy-16-ene-20-cyclopropyl-23,24-E-ene-26,27-hexafluoro-19-nor-cholecalciferol (250 mg) tetrabutylammonium fluoride (4 mL, 4 mmol, 1M solution in THF) was added, at room temperature. The mixture was stirred for 24 h. diluted with AcOEt (25 mL) and washed with water (5×20 mL), brine (20 mL) and dried over Na₂SO₄. The residue (270 mg) after evaporation of the solvent was purified by FC (10 g, 50% AcOEt in hexane and AcOEt) to give the titled compound (27) (157 mg, 0.30 mmol, 70%). [α]³_{D}=+63.3 c 0.45, EtOH. UV % max (EtOH): 243 nm (ε30821251 nm (ε 36064), 260 nm (F 24678); ¹H NMR (CDCl₃): 6.29 (1H, d, J=11.3 Hz), 6.24 (1H, dt, J=15.9, 6.4 Hz), 5.92 (1H, d, J=11.1 Hz), 5.61 (1H, d, J=15.7 Hz), 5.38 (1H, m), 4.13 (1H, m), 4.05 (1H, m), 2.88 (1H, s), 2.82-1.34 (19H, m), 0.770 (3H, s), 0.80-0.36 (4H, m); MS HRES Calculated for C₂₇H₃₄O₃F₆ M+H 521.2485. Observed M+H 521.2489.

### Example 34

### Synthesis of 1α,25-Dihydroxy-16-ene-20-cyclopropyl-23,24-E-ene-26,27-hexafluoro-cholecalciferol (28)

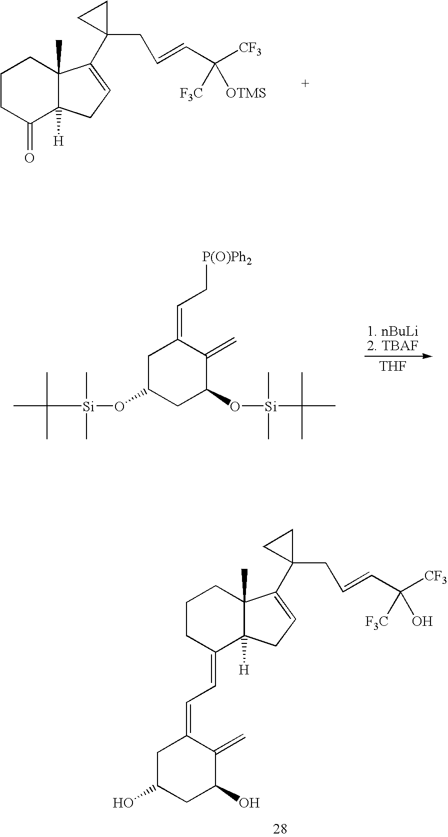

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (525 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2E-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 2.5 h diluted with hexane (35 mL) washed brine (30 mL) and dried over Na₂SO₄. The residue (760 mg) after evaporation of the solvent was purified by FC (15 g, 10% AcOEt in hexane) to give a mixture of 1α,3β-Di(tert-Butyl-dimethyl-silanyloxy)₂₅-trimethylsilanyloxy-16-ene-20-cyclopropyl-23,24-E-ene-26,27-hexafluoro-cholecalciferol and 1α,3β-Di(tert-Butyl-dimethyl-silanyloxy)-25-hydroxy-16-ene-20-cyclopropyl-23,24-E-ene-26,27-hexafluoro-cholecalciferol (274 mg). To the mixture of 1α,3β-Di(tert-Butyl-dimethyl-silanyloxy)-25-trimethylsilanyloxy-16-ene-20-cyclopropyl-23,24-E-ene-26,27-hexafluoro-cholecalciferol and 1α,3β-Di(tert-Butyl-dimethyl-silanyloxy)-25-hydroxy-16-ene-20-cyclopropyl-23,24-E-ene-26,27-hexafluoro-cholecalciferol (274 mg) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (280 mg) after evaporation of the solvent was purified by FC (15 g, 50% AcOEt in hexane and AcOEt) to give the titled compound (28) (167 mg, 0.31 mmol, 73%). [α]³_{D}=+18.3 c 0.41, EtOH. UV λmax (EtOH): 207 nm (c 17778), 264 nm (ε 15767); ¹H NMR (CDCl₃): 6.36 (1H, d, J=11.1 Hz), 6.24 (1H, dt, J=15.7, 6.7 Hz), 6.07 (1H, d, J=11.3 Hz), 5.60 (1H, d, J=15.5 Hz), 5.35 (1H, m), 5.33 (1H, s), 5.00 (1H, s), 4.44 (1H, m), 4.23 (1H, m), 3.14 (1H, s), 2.80 (1H, m), 2.60 (1H, m), 2.40-1.40 (15H, m), 0.77 (3H, s), 0.80-0.36 (4H, m); MS HRES Calculated for C₂₈H₃₄O₃F₆ M+H 533.2485. Observed M+H 533.2483.

### Example 35

### Synthesis of (3aR,4S,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-ol

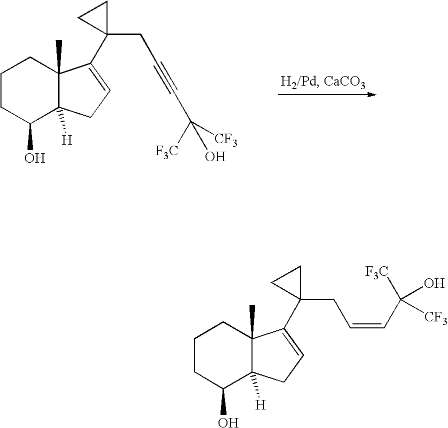

The mixture of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol (300 mg, 0.76 mmol), ethyl acetate (5 mL), hexane (12 mL), absolute ethanol (0.5 mL) quinoline (30 μL) and Lindlar catalyst (75 mg, 5% Pd on CaCO₃) was hydrogenated at room temperature for 2 h. The reaction mixture was filtered through a celite pad and the pad was washed with AcOEt. The solvent was evaporated to give the titled compound (257 mg, 0.65 mmol, 87%). [α]²⁸_{D}=+1.8 c 0.61, CHCl₃. ¹H NMR (CDCl₃): 6.08 (1H, dt, 3=12.3, 6.7 Hz), 5.47 (1H, m), 5.39 (1H, d, J=12.1 Hz), 4.15 (1H, br. s), 3.28 (1H, s), 2.52-1.34 (12H, m), 1.16 (3H, s), 0.78-0.36 (4H, m); ¹³C NMR (CDCl₃): 156.66(0), 141.77(1), 126.51(1), 122.79(0, q, J=285 Hz), 115.77 (1), 69.59(1), 55.41(1), 47.28(0), 36.44(2), 35.90 (2), 33.75(2), 30.22(2), 20.89(0), 19.41(3), 17.94(2), 12.05(2), 11.11(2); MS HRES Calculated for C₁₉H₂₄O₂F₆ M+H 399.1753. Observed M+H 399.1757.

### Example 36

### Synthesis of (3aR,7aR-7a-Methyl-1-[1-(5,5,5-trifluoro-4-trifluoromethyl-4-trimethylsilanyloxy-pen-2Z-enyl)-cyclopropyl]-3a,4,5,6,7,7a-hexahydro-3H-inden-4-one

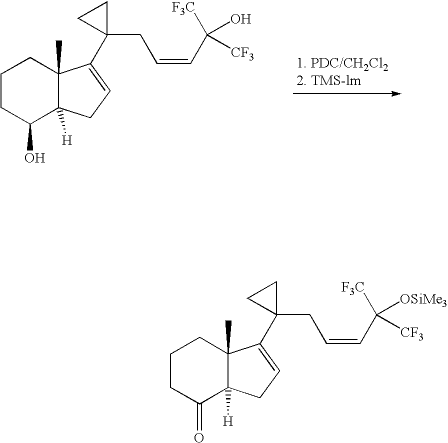

To a stirred suspension of (3aR,4S,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-ol (617 mg, 1.55 mmol) and Celite (2.0 g) in dichloromethane (10 mL) at room temperature wad added pyridinium dichromate (1.17 g, 3.1 mmol). The resulting mixture was stirred for 2.5 h filtered through silica gel (5 g), and then silica gel pad was washed with 20% AcOEt in hexane. The combined filtrate and washes were evaporated, to give a crude (3aR,7aR)-7a-Methyl-1-[1-(5,5,5-trifluoro-4-hydroxy-4-trifluoromethyl-pentenyl)-cyclopropyl]-3a,4,5,6,7,7a-hexahydro-3H-inden-4-one (600 mg, 1.51 mmol, 98%). To a stirred solution of (3aR,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-one (600 mg, 1.51 mmol) in dichloromethane (15 mL) at room temperature was added trimethylsilyl-imidazole (1.76 mL, 12.0 mmol). The resulting mixture was stirred for 1.0 h filtered through silica gel (10 g) and the silica gel pad was washed with 10% AcOEt in hexane. Combined filtered and washes were evaporated to give the titled compound (640 mg, 1.37 mmol, 88%). [α]²⁸_{D}=−0.2 c 0.55, CHCl₃. ¹H NMR (CDCl₃): 5.97 (1H, dt, J=12.2, 6.2 Hz), 5.40 (1H, m), 5.38 (1H, d, J=12.2 Hz), 2.82 (1H, dd, J=10.7, 6.6 Hz), 2.60-1.74 (10H, m), 0.89 (3H, s), 0.75-0.36 (4H, m), 0.21 (9H, s); ¹³C NMR (CDCl₃): 210.56(0), 154.30(0), 139.28(1), 125.81(1), 122.52(0, q, J=289 Hz), 118.17 (1), 64.11(1), 53.69(0), 40.43(2), 35.51(2), 34.85(2), 26.94(2), 24.07(2), 20.89(0), 18.39(3), 12.26(2), 10.61(2), 1.43 (3); MS HRES Calculated for C₂₂H₃₀O₂F₆Si M+H 469.1992. Observed M+H 469.1992.

### Example 37

### Synthesis of 1α,25-Dihydroxy-16-ene-20-cyclopropyl-23,24-Z-ene-26,27-hexafluoro-19-nor-cholecalciferol (29)

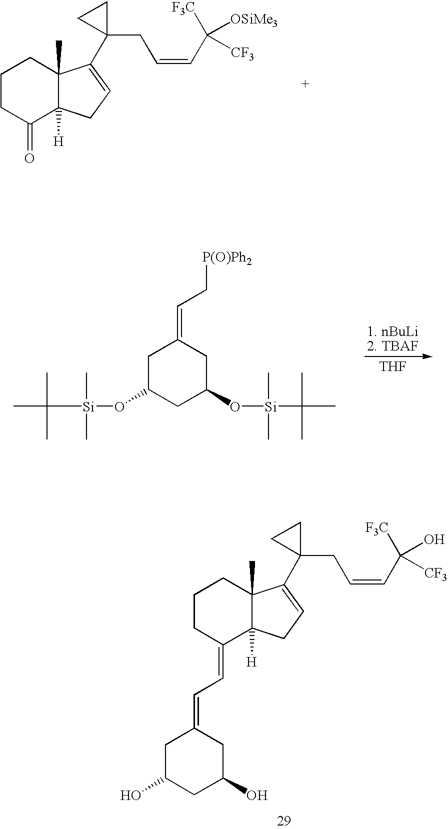

To a stirred solution of a (1R,3R)-1,3-bis-((tert-butyldimethyl)silanyloxy)-5-[2-(diphenylphosphinoyl)ethylidene]-cyclohexane (514 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2Z-enyl)-cyclopropyl]-3a,4,5,6,7,7a-hexahydro-3H-inden-4-one (194 mg, 0.41 mmol, in tetrahydrofurane (2 mL) was added dropwise. The reaction mixture was stirred at −72° C. for 3.0 h diluted with hexane (35 mL) washed brine (30 mL) and dried over Na₂SO₄. The residue (750 mg) after evaporation of the solvent was purified by FC (15 g, 10% AcOEt in hexane) to give a mixture of 1α,3β-Di(tert-Butyl-dimethyl-silanyloxy)-25-trimethylsilanyloxy-16-ene-20-cyclopropyl-23,24-Z-ene-26,27-hexafluoro-19-nor-cholecalciferol and 1α,3β-Di(tert-Butyl-dimethyl-silanyloxy)-25-hydroxy-16-ene-20-cyclopropyl-23,24-Z-ene-26,27-hexafluoro-19-nor-cholecalciferol (230 mg). To the mixture of 1α,3β-Di(tert-Butyl-dimethyl-silanyloxy)-25-trimethylsilanyloxy-16-ene-20-cyclopropyl-23,24-Z-ene-26,27-hexafluoro-19-nor-cholecalciferol and 1α,3β-Di(tert-Butyl-dimethyl-silanyloxy)-25-hydroxy-16-ene-20-cyclopropyl-23,24-Z-ene-26,27-hexafluoro-19-nor-cholecalciferol (230 mg) tetrabutylammonium fluoride (4 mL, 4 mmol, 1M solution in THF) was added, at room temperature. The mixture was stirred for 40 h. diluted with AcOEt (25 mL) and washed with water (5×20 mL), brine (20 mL) and dried over Na₂SO₄. The residue (260 mg) after evaporation of the solvent was purified by FC (10 g, 50% AcOEt in hexane and AcOEt) to give the titled compound (29) (1327 mg, 0.25 mmol, 62%). [α]²⁸_{D}=+53.6 c 0.33, EtOH. UV λmax (EtOH): 243 nm (ε26982251 nm (ε 32081), 260 nm (ε 21689); ¹H NMR (CDCl₃): 6.29 (1H, d, J=10.7 Hz), 6.08 (1H, dt, J=12.5, 6.7 Hz), 5.93 (1H, d, J=11.1 Hz), 5.46 (1H, m), 5.40 (1H, d, J=12.7 Hz)), 4.12 (1H, m), 4.05 (1H, m), 3.14 (1H, s), 2.80-1.40 (19H, m), 0.77 (3H, s), 0.80-0.36 (4H, m); MS HRES Calculated for C₂₇H₃₄O₃F₆ M+H 521.2485. Observed M+H 521.2487.

### Example 38

### Synthesis of 1α,25-Dihydroxy-16-ene-20-cyclopropyl-23,24-Z-ene-26,27-hexafluoro-cholecalciferol (30)

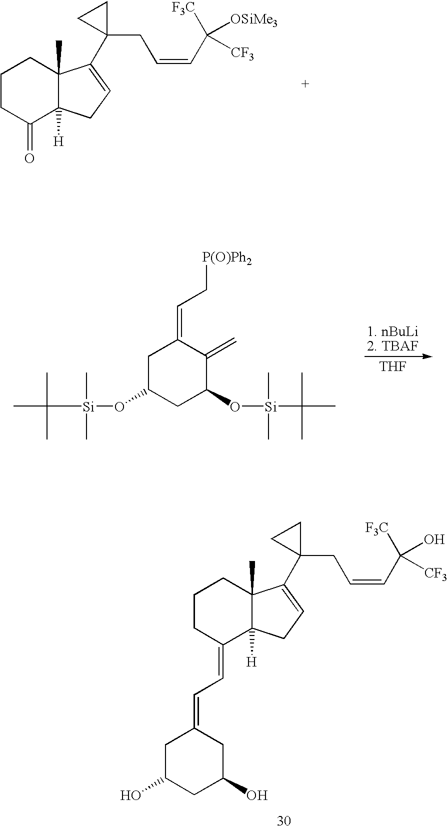

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (525 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2Z-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 2.5 h diluted with hexane (35 mL) washed brine (30 mL) and dried over Na₂SO₄. The residue (680 mg) after evaporation of the solvent was purified by FC (15 g, 10% AcOEt in hexane) to give a mixture of 1α,3β-Di(tert-Butyl-dimethyl-silanyloxy)-25-trimethylsilanyloxy-16-ene-20-cyclopropyl-23,24-Z-ene-26,27-hexafluoro-cholecalciferol and 1α,3β-Di(tert-Butyl-dimethyl-silanyloxy)-25-hydroxy-16-ene-20-cyclopropyl-23,24-Z-ene-26,27-hexafluoro-cholecalciferol (310 mg). To the mixture of 1α,3β-Di(tert-Butyl-dimethyl-silanyloxy)-25-trimethylsilanyloxy-16-ene-20-cyclopropyl-23,24-Z-ene-26,27-hexafluoro-cholecalciferol and 1α,3β-Di(tert-Butyl-dimethyl-silanyloxy)-25-hydroxy-16-ene-20-cyclopropyl-23,24-Z-ene-26,27-hexafluoro-cholecalciferol (310 mg) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (370 mg) after evaporation of the solvent was purified by FC (10 g, 50% AcOEt in hexane and AcOEt) to give the titled compound (30) (195 mg, 0.37 mmol, 85%). [α]³⁰_{D}=+9.4 c 0.49, EtOH. UV λmax (EtOH): 262 nm (ε 11846); ¹H NMR (CDCl₃): 6.36 (1H, d, J=11.1 Hz), 6.08 (2H, m), 5.44 (1H, m), 5.40 (1H, d, J=12.3 Hz), 5.32 (1H, s), 5.00 (1H, s), 4.43 (1H, m), 4.23 (1H, m), 3.08 (1H, s), 2.80 (1H, m), 2.60 (1H, m), 2.55-1.40 (15H, m), 0.77 (3H, s), 0.80-0.34 (4H, m); MS HRES Calculated for C₂₈H₃₄O₃F₆ M+H 533.2485. Observed M+H 533.2502.

### Example 39

### Synthesis of 1α,25-Dihydroxy-16-ene-20-cyclopropyl-19-nor-cholecalciferol (31)

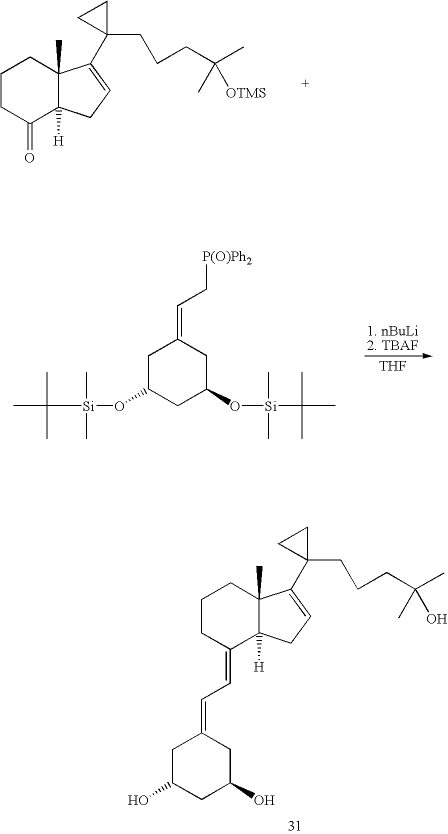

To a stirred solution of a (1R,3R)-1,3-bis-((tert-butyldimethyl)silanyloxy)-5-[2-(diphenylphosphinoyl)ethylidene]-cyclohexane (697 mg, 1.22 mmol) in tetrahydrofurane (9 mL) at −78° C. was added n-BuLi (0.77 mL, 1.23 mmol). The resulting mixture was stirred for 15 min and solution of (3aR,7aR)-7a-Methyl-1-[1-(4-methyl-4-trimethylsilanyloxy-pentyl)-cyclopropyl]-3a,4,5,6,7,7a-hexahydro-3H-inden-4-one (220 mg, 0.61 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (900 mg) after evaporation of the solvent was purified by FC (15 g, 10% AcOEt in hexane) to give 1α,3β-Di(tert-Butyl-dimethyl-silanyloxy)-25-trimethylsilanyloxy-16-ene-20-cyclopropyl-19-nor-cholecalciferol (421 mg, 0.59 mmol). To the 1α,3β-Di(tert-Butyl-dimethyl-silanyloxy)-25-trimethylsilanyloxy-16-ene-20-cyclopropyl-26,27-hexadeutero-19-nor-cholecalciferol (421 mg, 0.59 mmol) tetrabutylammonium fluoride (4 mL, 4 mmol, 1M solution in THF) was added, at room temperature. The mixture was stirred for 40 h. diluted with AcOEt (25 mL) and washed with water (5×20 mL), brine (20 mL) and dried over Na₂SO₄. The residue (450 mg) after evaporation of the solvent was purified by FC (15 g, 50% AcOEt in hexane and AcOEt) to give the titled compound (31) (225 mg, 0.54 mmol, 89%). [α]²⁹_{D}=+69.5 c 0.37, EtOH. UV % max (EtOH): 243 nm (ε27946.251 nm (ε 33039), 261 nm (ε 22701); ¹H NMR (CDCl₃): 6.30 (1H, d, J=11.3 Hz), 5.93 (1H, d, J=11.3 Hz), 5.36 (1H, m), 4.12 (1H, m), 4.04 (1H, m), 2.75 (2H, m), 2.52-1.04 (22H, m), 1.18 (6H, s), 0.79 (3H, s), 0.65-0.26 (4H, m); ¹³C NMR (CDCl₃): 157.16(0), 142.33(0), 131.25(0), 124.73(1), 123.76(1), 115.50(1), 71.10(0), 67.39(1), 67.19(1), 59.47(1), 50.12(0), 44.60(2), 43.84(2), 42.15(2), 38.12(2), 37.18(2), 35.57(2), 29.26(3), 29.11(2), 29.08(3), 28.48(2), 23.46(2), 22.26(2), 21.27(0), 17.94(3), 12.70(2), 10.27(2); MS HRES Calculated for C₂₇H₄₂O₃ M+H 415.3207. Observed M+H 415.3207.

### Example 40

### Synthesis of 1α,25-Dihydroxy-16-ene-20-cyclopropyl-cholecalciferol (32)

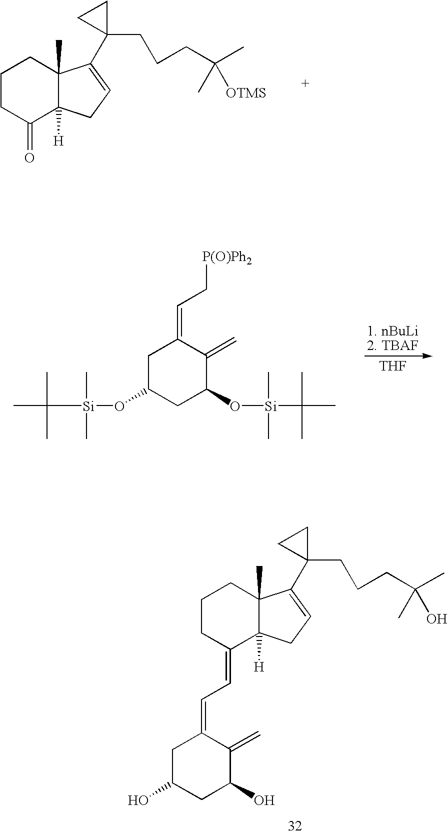

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (675 mg, 1.16 mmol) in tetrahydrofurane (8 mL) at −78° C. was added n-BuLi (0.73 mL, 1.17 mmol). The resulting mixture was stirred for 15 min and solution of (3aR,7aR)-7a-Methyl-1-[1-(4-methyl-4-trimethylsilanyloxy-pentyl)-cyclopropyl]-3a,4,5,6,7,7a-hexahydro-3H-inden-4-one (210 mg, 0.58 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (850 mg) after evaporation of the solvent was purified by FC (15 g, 10% AcOEt in hexane) to give 1α,3β-Di(tert-Butyl-dimethyl-silanyloxy)-25-trimethylsilanyloxy-16-ene-20-cyclopropyl-cholecalciferol (382 mg, 0.53 mmol). To the 1α,3β-Di(tert-Butyl-dimethyl-silanyloxy)-25-trimethylsilanyloxy-16-ene-20-cyclopropyl-cholecalciferol (382 mg, 0.53 mmol) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (380 mg) after evaporation of the solvent was purified by FC (15 g, 50% AcOEt in hexane and AcOEt) to give the titled compound (32) (204 mg, 0.48 mmol, 83%). [α]²⁹_{D}=+16.1 c 0.36, EtOH. UV λmax (EtOH): 208 nm (ε 17024), 264 nm (ε 16028); ¹H NMR (CDCl₃): 6.37 (1H, d, J=11.3 Hz), 6.09 (1H, d, J=11.1 Hz), 5.33 (2H, m), 5.01 (1H, s), 4.44 (1H, m), 4.23 (1H, m), 2.80 (1H, m), 2.60 (1H, m), 2.38-1.08 (20H, m), 1.19 (6H, s), 0.79 (3H, s), 0.66-0.24 (4H, m); ¹³C NMR (CDCl₃): 157.07(0), 147.62(0), 142.49(0), 133.00(0), 124.90(1), 124.73(1), 117.19(1), 111.64(2), 71.10(1), 70.70(0), 66.88(1), 59.53(1), 50.28(0), 45.19(2), 43.85(2), 42.86(2), 38.13(2), 35.59(2), 29.27(2), 29.14(3), 28.65(2), 23.57(2), 22.62(2), 21.29(0), 17.84(3), 12.74(2), 10.30(2); MS HRES Calculated for C₂₉H₄₂O₃ M+Na 449.3026. Observed M+Na 449.3023.

### Example 41

### Synthesis of 1,25-Dihydroxy-21-(2R,3-dihydroxy-3-methyl-butyl)-20R-Cholecalciferol (33)

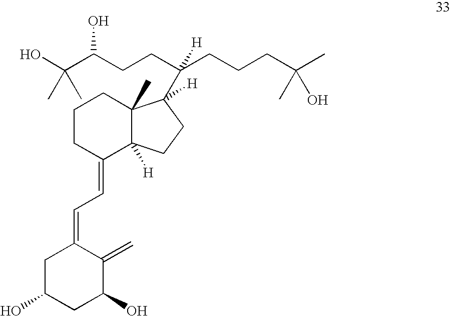

### [1R,3aR,4S,7aR]-2(R)-[4-(1,1-dimethylethyl)dimethyl-silanyloxy)-7a-methyl-octahydro-inden-1-yl]-6-methyl-heptane-1,6-diol (34) and [1R,3aR,4S,7aR]-2(S)-[(1,1-dimethylethyl)dimethyl-silanyloxy)₇a-methyl-octahydro-inden-1-yl]-6-methyl-heptan-1,6-diol (35)

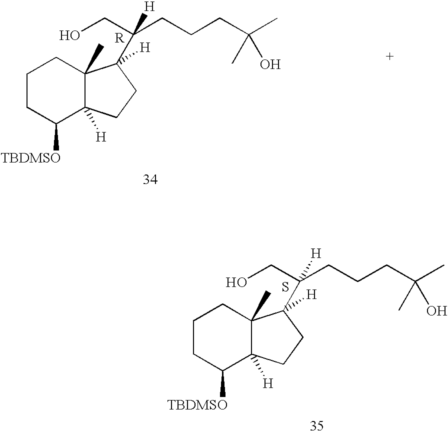

A solution of the alkenol in tetrahydrofuran (9 mL) was cooled in an ice bath and a 1 M solution of borane-THF in tetrahydrofuran (17 mL) was added dropwise in an originally effervescent reaction. The solution was stirred overnight at room temperature, re-cooled in an ice bath water (17 mL) was added dropwise followed by sodium percarbonate (7.10 g, 68 mmol). The mixture was immersed into a 50° C. bath and stirred for 70 min to generate a solution. The two-phase system was allowed to cool then equilibrated with 1:1 ethyl acetate-hexane (170 mL). The organic layer was washed with water (2×25 mL) then brine (20 mL), dried and evaporated to leave a colorless oil (2.76 g). This material was passed through a short flash column using 1:1 ethyl acetate-hexane and silica gel G. The effluent, obtained after exhaustive elution, was evaporated, taken up in ethyl acetate, filtered and chromatographed on the 2×18″ 15-20μ silica YMC HPLC column using 2:1 ethyl acetate-hexane as mobile phase and running at 100 mL/min. Isomer 34 emerged at an effluent maximum of 2.9 L, colorless oil, 1.3114 g, [α]_{D}+45.20 (methanol, c 0.58; ¹H NMR δ −0.002 (3H, s), 0.011 (3H, s), 0.89 (9H, s), 0.93 (3H, s), 1.17 (1H, m), 1.22 (6H, s), 1.25-1.6 (16H, m), 1.68 (1H, m), 1.80 (2H, m), 1.89 (1H, m), 3.66 (1H, dd, J=4.8 and 11 Hz), 3.72 (1H, dd, J=3.3 and 11 Hz), 4.00 (1H, m); LR-ES(−) m/z 412 (M), 411 (M−H); HR-ES(+): calcd for (M+Na) 435.3265, found: 435.3269.

Isomer 35 at was eluted at an effluent maximum of 4.9 L, colorless oil, 0.8562 g that crystallized upon prolonged standing: mp 102-3°, [α]_{D}+25.2° (methanol, c 0.49); ¹H NMR δ −0.005 (3H, s), 0.009 (3H, s), 0.89 (9H, s), 0.93 (3H, s), 1.16 (1H, m), 1.22 (6H, s), 1.3-1.5, (14H, m), 1.57 (2H, m), 1.67 (1H, m), 1.80 (2H, m), 1.91 (1H, m), 3.54 (1H, dd, J=4.8 and 11 Hz), 3.72 (1H, dd, J=2.9 and 11 Hz), 4.00 (1H, m); LR-ES(−) m/z 412 (M), 411 (M−H). Anal. Calcd for C₂₄H₄₈O₃Si: C, 69.84, H, 11.72; found: C, 69.91; H, 11.76.

### [1R,3aR,4S,7aR]-(R-[4-(tert-Butyl-dimethyl-silanyloxy)-7a-methyl-octahydro-inden-1-yl]-7-iodo-2-methyl-heptan-2-ol (36)

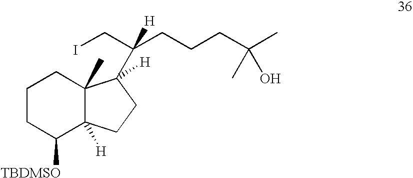

A stirred mixture of triphenylphosphine (0.333 g, 1.27 mmol) and imidazole (0.255 g, 3 mmol) in dichloromethane (3 mL) was cooled in an ice bath and iodine (0.305 g, 1.20 mmol) was added. This mixture was stirred for 10 min then a solution of 34 (0.4537 g, 1.10 mmol) in dichloromethane (3 mL) was added dropwise over a 10 min period. The mixture was stirred in the ice bath for 30 min then at ambient temperature for 2¾/h. TLC (1:1 ethyl acetate-hexane) confirmed absence of educt. A solution of sodium thiosulfate (0.1 g) in water (5 mL) was added, the mixture equilibrated and the organic phase washed with 0.1 N sulfuric acid (10 mL) containing a few drops of brine then with 1:1 water-brine (2×10 mL), once with brine (10 mL) then dried and evaporated. The residue was purified by flash chromatography using 1:9 ethyl acetate-hexane as mobile phase to furnish 36 as a colorless syrup, 0.5637 g, 98%: ¹H NMR δ −0.005 (3H, s), 0.010 (3H, s), 0.89 (9H, s), 0.92 (3H, s), 1.23 (6H, s), 1.1-1.6 (16H, m), 1.68 (1H, m), 1.79 (2H, m), 1.84 (1H, m), 3.37(1H, dd, J=4 and 10 Hz), 3.47 (1H, dd, J=3 and 10 Hz), 4.00 (1H, m); LR-EI(+) m/z 522 (M), 465 (M−C₄H₉), 477 (M−C₄H₉—H₂O); HR-EI(+): calcd for C₂₄H₄₇IO₂Si: 522.2390, found: 522.2394.

### [1R,3aR,4S,7aR]-6(S)-[4-(tert-Butyl-dimethyl-silanyloxy)-7a-methyl-octahydro-inden-1-yl]-2-methyl-non-8-yn-2-ol (37)

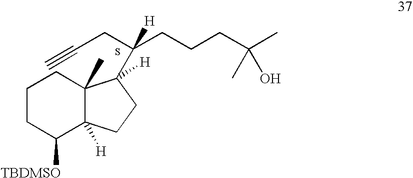

Lithium acetylide DMA complex (0.110 g, 1.19 mmol) was added to a solution of 36 (0.2018 g (0.386 mmol) in dimethyl sulfoxide (1.5 mL) and tetrahydrofuran (0.15 mL). The mixture was stirred overnight. TLC (1:4 ethyl acetate-hexane) showed a mixture of two spots traveling very close together (Rf 0.52 and 0.46). Fractions at the beginning of the eluted band contained pure alkenol, which is the elimination product of 36, and was produced as the major product. Fractions at the end of the elution band, however, were also homogeneous and gave the desired acetylene 37 upon evaporation. The NMR spectra of 37 and its 6-epimer which served for identification were previously reported.

### [1R,3aR,4S,7aR]-7-Benzenesulfonyl-6(S)-[4-(tert-butyl-dimethyl-silanyloxy)-7a-methyl-octahydro-inden-1-yl]-2-methyl-heptan-2-ol (38)

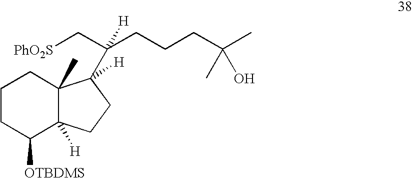

A mixture of 37b (0.94 g, 1.8 mmol), sodium benzenesulfinate (2.18 g, 13 mmol) and N,N-dimethylformamide (31.8 g) was stirred at room temperature for 12 h, then in a 40° C. bath for ca.6 h until all educt was converted as shown by TLC (1:4 ethyl acetate-hexane). The solution was equilibrated with 1:1 ethyl acetate-hexane (120 mL) and 1:1 brine-water (45 mL). The organic layer was washed with water (4×25 mL) brine (10 mL), then dried and evaporated to leave a colorless oil, 1.0317 g. This material was flash-chromatographed using a stepwise gradient (1:9, 1:6, 1:3 ethyl acetate-hexane) to give a colorless oil, 0.930 g, 96%: 300 MHz ¹H NMR 6-0.02 (3H, s), 0.00 (3H, s), 0.87 (9H, s), 0.88 (3H, s), 1.12 (1H, m), 1.20 (6H, s), 1.2-1.8 (18H, m), 1.81 (1H, m), 3.09 (2H, m), 3.97 (1H, brs), 7.59 (3H, m), 7.91 2H, m).

### [1R,3aR,4S,7aR]-1-(1(S)-Benzenesulfonylmethyl-5-methyl-5-trimethylsilanyloxy-hexyl)-4-(tert-butyldimethyl-silanyloxy)-7a-methyl-octahydro-indene (39)

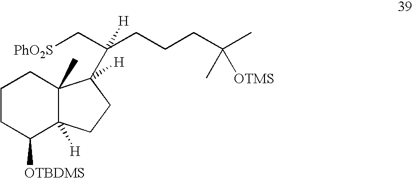

1-(Trimethylsilyl)imidazole (1 mL) was added to a solution of 38 (0.8 g) in cyclohexane (10 mL) and stirred overnight then flash-chromatographed using a stepwise gradient of hexane, 1:39 and 1:19 ethyl acetate-hexane. The elution was monitored by TLC (1:4 ethyl acetate-hexane) leading to 39 as a colorless syrup, 0.7915 g: 300 MHz ¹H NMR δ0.00 (3H, s), 0.02 (3H, s), 0.12 (9H, s), 0.90 (12H, s, t-butyl+7a-Me), 1.16 (1H, m), 1.20 (6H, s), 1.2-1.6 (15H, m), 1.66-1.86 (3H, m), 3.10 (2H, m), 4.00 (1H, brs), 7.56-7.70 (3H, m), 7.93 (2H, m).

### [1R,3aR,4S,7aR]-6(R)-[4-(tert-Butyl-dimethyl-silanyloxy)-7a-methyl-octahydro-inden-1-yl]-2,10-dimethyl-undecane-2,3(R),10-triol (40)

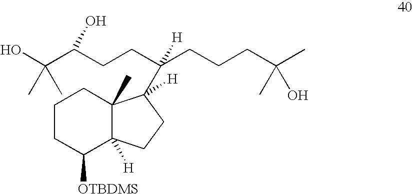

A solution of 39 (0.7513 g, 1.23 mmol) and diol (0.508 g, 1.85 mmol) in tetrahydrofuran (28 mL) was cooled to −35° C. then 2.5 M butyllithium in hexane (2.75 mL) was added dropwise. The temperature was allowed to rise to −20° C. and maintained at that temperature for 6 h or until the educt was consumed. Reaction progress was monitored by TLC (1:4 ethyl acetate-hexane) exhibiting the educt (Rf 0.71) and the two epimeric diols (Rf 0.09 and 0.12). Toward the end of the reaction period the temperature was increased briefly to 0° C., lowered again to −10, then saturated ammonium chloride (25 mL) was added followed by ethyl acetate (50 mL) and enough water to dissolve the precipitated salts. The resulting aqueous phase was extracted with ethyl acetate (15 mL). The combined extracts were washed with brine (15 mL), dried and evaporated. The resulting syrup was flash-chromatographed using a stepwise gradient of 1:9, 1:6, 1:4 and 1:1 ethyl acetate-hexane to give 39a as a colorless syrup, 0.8586 g. This material was dissolved in a mixture of tetrahydrofuran (30 mL) and methanol (18 mL), then 5% sodium amalgam (20 g) was added. The reductive de-sulfonylation was complete after stirring of the mixture for 14 h. Progress of the reaction was monitored by TLC (1:1 ethyl acetate-hexane) which showed the disappearance of the epimeric diols (Rf 0.63 and 0.74) and the generation of 40a (Rf 0.79) and the partially de-silylated analog 40 (Rf 0.16). The mixture was diluted with methanol (20 mL), stirred for 3 min, then ice (20 g) was added, stirred for 2 min and the supernatant decanted into a mixture containing saturated ammonium chloride (50 mL). The residue was repeatedly washed with small amounts of tetrahydrofuran that was also added to the salt solution, which was then equilibrated with ethyl acetate (80 mL). The aqueous layer was re-extracted once with ethyl acetate (20 mL), the combined extracts were washed with brine (10 mL) then dried and evaporated. The resulting colorless oil containing both 40a and 40 was dissolved in 10 mL of a 1 N oxalic acid solution in methanol (prepared from the dihydrate) effecting the selective hydrolysis of the trimethylsilyl ether within minutes. Calcium carbonate (1 g) was added and the suspension stirred overnight, then filtered. The solution was evaporated and the resulting residue flash-chromatographed using a stepwise gradient of 1:4, 1:2, 1:1 and 2:1 ethyl acetate-hexane giving a residue of the triol 40 that crystallized in very fine branching needles from acetonitrile, 0.45 g: mp 94-95° C., [α]D+44.1° (methanol, c 0.37); 400 MHz ¹H NMR δ −0.005 (3H, s), 0.007 (3H, s), 0.89 (9H, s), 0.92 (3H, s), 1.15 (1H, m), 1.16 (3H, s), 1.21 (9H, s), 1.2-1.6 (19H, m), 1.67 (1H, m), 1.79 (2H, m), 1.90 (2H, m), δ 2.06 (1H, m), 3.31 (1H, brd, J=10 Hz), 4.00 (1H, brs), LR-ES(−) m/z: 533 (M+Cl), 497 (M−H); HR-ES(+): Calcd for C₂₉H₅₈O₄Si+Na: 521.3996, found: 521.4003. Anal Calcd for C₂₉H₅₈O₄Si: C, 69.82, H, 11.72; found: C, 69.97; H, 11.65.

### [1R,3aR,4S,7aR]-6(R)-(4-Hydroxy-7a-methyl-octahydro-inden-1-yl)-2,10-dimethyl-undecane-2,3(R),10-triol (41)

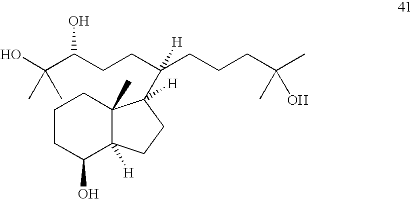

A stirred solution of the triol 40 (0.4626 g, 0.927 mmol) in acetonitrile (10 mL) and dioxane (0.7 mL) was cooled to 10° C. and a fluorosilicic acid solution (2 mL) was added dropwise. The cooling bath was removed, the 2-phase system further diluted with acetonitrile (2 mL) then stirred at room temperature for 3¼h. The disappearance of educt was monitored by TLC (ethyl acetate). The mixture was equilibrated with water (10 mL) and ethyl acetate (30 mL). The aqueous phase was re-extracted with ethyl acetate (2×20 mL), the combined extracts were washed with water (5 mL) and brine (10 mL), then 1:1 brine-saturated sodium hydrogen carbonate solution and dried. The residue was purified by flash-chromatography using a step-wise gradient from 1:1 to 2:1 ethyl acetate-hexane and neat ethyl acetate to give a residue that was taken up in 1:1 dichloromethane-hexane, filtered and evaporated to furnish amorphous solids, 0.3039 g (85%): [α]_{D}+42.60 (methanol, c 0.48); ¹H NMR (DMSO-d₆): δ 0.87 (3H, s), 0.97 (3H, s), 1.02 (3H, s), 1.04 (6H, s), 1.1-1.4 (18H, m), 1.5-1.8 (4H, m), 1.84 (1H, m), 2.99 (1H, dd, J=6 and 10 Hz), 3.87 (1H, brs), 4.02 (1H, s, OH), 4.05 (1H, s, OH), 4.16 (1H, d, OH, J=3.6 Hz), 4.20 (1H, d, OH, J=6.4 Hz); LR-ES(+): m/z 384 (M), 383 (M−H); HR-ES(+): Calcd for (M+Na) 407.3132, found: 407.3134.

### [1R,3aR,4S,7aR]-1-{5-Hydroxy-5-methyl-1 (R)-[2-(2,2,5,5-tetramethyl-[1,3]dioxolan-4(R)-yl)-ethyl]-hexyl}-7a-methyl-octahydro-inden-4-ol (42)

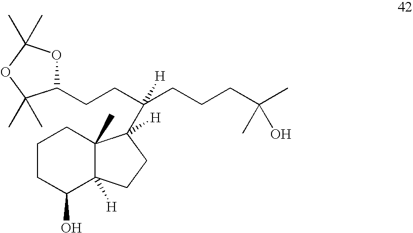

A solution of the tetraol 40 (0.2966 g, 0.771 mmol) and pyridinium tosylate (100 mg) in acetone (8 mL) and 2,2-dimethoxypropane (8 mL) was kept at room temperature for 12 h. TLC analysis (ethyl acetate) showed the absence of educt (Rf 0.21) and two new spots with Rf 0.82 and 0.71, the former the expected 42 and the latter assumed to be the methylacetal. The reaction mixture was diluted with water (5 mL) and stirred for 10 min. At that time only the spot with higher Rf value was observed. The mixture was neutralized with sodium hydrogen carbonate (0.5 g) then equilibrated with ethyl acetate (50 mL) and brine (5 mL). The organic layer was washed with water (5 mL) and brine (5 mL) then dried and evaporated to leave a sticky residue (0.324 g) that was used directly in the next step: 300 MHz ¹H NMR: δ 0.94 (3H, s), 1.10 (3H, s), 1.20 (1H, m), 1.22 (6H, s), 1.25 (3H, s), 1.34 (3H, s), 1.41 (3H, s), 1.2-1.65 (20H, m), 1.78-1.86 (3H, m), 1.93 (1H, m), 3.62 (1H, dd, J=4.6 and 8.3 Hz), 4.08 (1H, brs).

### [1R,3aR,4S,7aR]-Acetic acid 1-{5-hydroxy-5-methyl-1(R)-[2-(2,2,5,5-tetramethyl-[1,3]dioxolan-4(R)-yl)-ethyl]-hexyl}-7a-methyl-octahydro-inden-4-yl ester (43)

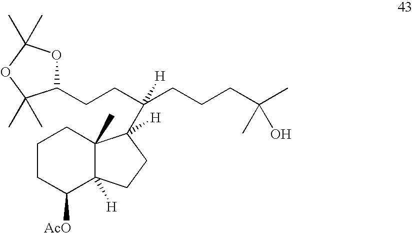

The residue obtained above was dissolved in pyridine (6.9 g) and further diluted with acetic anhydride (3.41 g). The mixture was allowed to stand at room temperature for 24 h, then in a 35° C. bath for ca. 10 h until the educt was no longer detectable (TLC, ethyl acetate). The mixture was diluted with toluene and evaporated. The residue was purified by flash chromatography (1:4 ethyl acetate-hexane) to give 43 as colorless syrup, 0.3452 g, 97%: ¹H NMR: δ 0.89 (3H, s), 1.10 (3H, s), 1.20 (1H, m), 1.22 (6H, s), 1.25 (3H, s), 1.33 (3H, s), 1.41 (3H, s), 1.25-1.6 (19H, m), 1.72 (1H, m), 1.82 (2H, m), 1.95 (1H, m), 2.05 (3H, s), 3.63 (1H, dd, J=4.4 and 8.4 Hz), 5.15 (1H, brs); LR-FAB(+) m/z 467 (M+H), 465 (M−H), 451 (M−Me).

### [1R,3aR,4S,7aR]-Acetic acid 1-[4(R),5-dihydroxy-1(R)-(4-hydroxy-4-methyl-pentyl)-5-methyl-hexyl]-7a-methyl-octahydro-inden-4-yl ester (44)

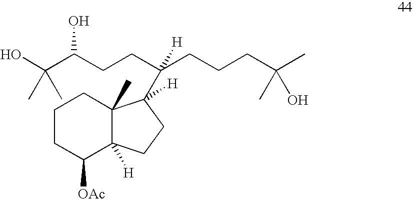

A solution of 43 (0.334 g, 0.716 mmol) in 80% acetic acid (2 mL) was kept in a 68° C. bath. TLC (ethyl acetate, Rf 0.33) monitored the progress of the hydrolysis. The educt was no longer detectable after 2.5 h. The mixture was evaporated then co-evaporated with a small amount of toluene to leave a colorless film (0.303 g) that was used directly in the next step: 300 MHz ¹H NMR: δ 0.89 (3H, s), 1.17 (3H, s), 1.22 (6H, s), 1.56 (3H, s), 1.1-1.6 (21H, m), 1.6-2.0 (5H, m), 2.04 (3H, s), 3.32 (1H, brd, J=10 Hz), 5.15 (1H, brs).

### [1R,3aR,4S,7aR]-Acetic acid 1-[4(R)-[dimethyl-(1,1,2-trimethyl-propyl)-silanyloxy]-5-hydroxy-1(R)-(4-hydroxy-4-methyl-pentyl)-5-methyl-hexyl]-7a-methyl-octahydro-inden-4-yl ester (45)

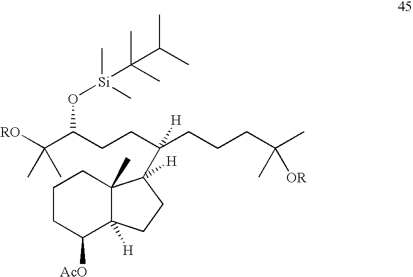

A solution of the triol 44 (0.30 g), imidazole (0.68 g, 10 mmol) and dimethylthexylsilyl chloride (1.34 g, 7.5 mmol) in N,N-dimethylformamide (6 g) was kept at room temperature. After 48 h 4-(N,N-dimethylamino)pyridine (15 mg) was added and the mixture stirred for an additional 24 h. Reaction progress was monitored by TLC (ethyl acetate; 24, Rf 0.83; 25a, Rf 0.38). The mixture was diluted with water (2 mL), stirred for 10 min then distributed between ethyl acetate (45 mL) and water (20 mL). The aqueous layer was extracted once with ethyl acetate (10 mL). The combined organic phases were washed with water (4×12 mL) and brine (8 mL) then dried and evaporated. The residual oil was purified by flash-chromatography using a stepwise gradient of 1:9 and 1:4 ethyl acetate-hexane to give 45 as colorless syrup. A small amount of unreacted educt (80 mg) was eluted with ethyl acetate. The syrupy 45 was used directly in the next step: 400 MHz ¹H NMR: δ 0.13 (3H, s), 0.14 (3H, s), 0.87 (6H, s), 0.91 (9H, m), 1.10 (1H, m), 1.14 (3H, s), 1.15 (3H, s), 1.21 (6H, s), 1.1-1.6 (19H, m), 1.6-1.9 (5H, m), 1.94 (1H, brd, J=12.8 Hz), 2.05 (3H, s), 3.38 (1H, brs), 5.15 (1H, brs).

### [1R,3aR,4S,7aR]-Acetic acid 1-[4(R-[dimethyl-(1,1,2-trimethyl-propyl)-silanyloxy]-5-methyl-1(R)-(4-methyl-4-trimethylsilanyloxy-pentyl)-5-trimethylsilanyloxy-hexyl]-7a-methyl-octahydro-indenyl ester (46)

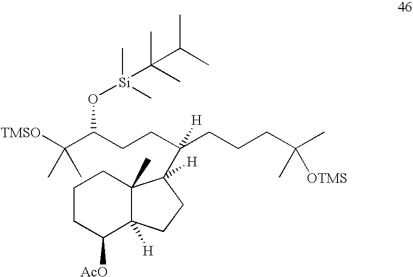

1-(Trimethylsilyl)imidazole (0.90 mL, 6.1 mmol) was added to a solution of 45 (0.2929 mg) in cyclohexane (6 mL) and stirred for 12 h, then flash-chromatographed (1:79 ethyl acetate-hexane) to yield 46 as colorless syrup (0.3372 g). The elution was monitored by TLC (1:4 ethyl acetate-hexane) leading to 46 as a colorless syrup, 0.7915 g: ¹H NMR δ: 0.074 (3H, s), 0.096 (3H, s), 0.103 (9H, s), 0.106 (9H, s), 0.82 (1H, m), 0.83 (6H, s), 0.88 (9H, m), 1.32 (3H, s), 1.20 (9H, s), 1.15-1.6 (17H, m), 1.6-1.9 (5H, m), 1.97 (1H, brd, J=12.8 Hz), 2.05 (3H, s), 3.27 (1H, m), 5.15 (1H, brs); LR-FAB(+) m/z: 712 (M), 711 (M−H), 697 (M−Me), 653 (M−AcO), 627 (M−C₆H₁₃).

### [1R,3aR,4S,7aR]-1-[4(R)-[Dimethyl-(1,1,2-trimethyl-propyl)-silanyloxy]-5-methyl-1(R)-(4-methyl-4-trimethylsilanyloxy-pentyl)-5-trimethylsilanyloxy-hexyl]-7a-methyl-octahydro-inden-4-ol (47)

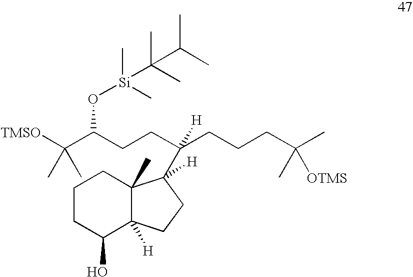

A stirred solution of 46 (0.335 mg, 0.47 mmol) in tetrahydrofuran (15 mL) was cooled in an ice-bath and a 1 M solution of lithium aluminum hydride in tetrahydrofuran (2 mL) was added dropwise. TLC (1:9 ethyl acetate-hexane) showed complete conversion 25b (Rf 0.61) to 26 (Rf 0.29) after 1.5 h. A 2 M sodium hydroxide solution (14 drops) was added, followed by water (0.5 mL) and ethyl acetate (30 mL). A small amount of Celite was added and, after stirring for 15 min, the liquid layer was filtered off. The solid residue was rinsed repeatedly with ethyl acetate and the combined liquid phases evaporated to leave a colorless syrup, that was taken up in hexane, filtered and evaporated to yield 26 (0.335 g) that was used without further purification: ¹H NMR δ: 0.075 (3H, s), 0.10 (21H, brs), 0.82 (1H, m), 0.84 (6H, s), 0.89 (6H,m), 0.93 (3H, s), 1.13 (3H, s), 1.20 (9H, s), 1.2-1.6 (16H, m), 1.6-1.7 (2H, m), 1.82 (3H, m), 1.95 (1H, brd, J=12.4 Hz), 3.27 (1H, m), 4.08 (1H, brs); LR-FAB(+) m/z: 585 (M−C₆H₁₃), 481 (M−TMSO); HR-ES(+) m/z: Calcd for C₃₇H₇₈O₄Si₃+Na: 693.5100 found: 693.5100.

### [1R,3aR,7aR]-1-[4(R)-[Dimethyl-(1,1,2-trimethyl-propyl)-silanyloxy]-5-methyl-1(R)-(4-methyl-4-trimethylsilanyloxy-pentyl)-5-trimethylsilanyloxy-hexyl]-7a-methyl-octahydro-inden-4-one (48)

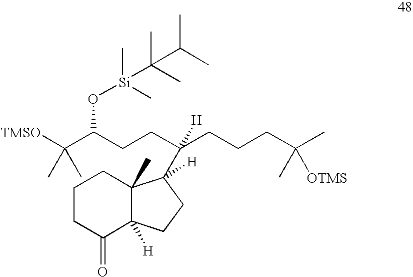

Celite (0.6 g) was added to a stirred solution of 47 (0.310 g, 0.462 mmol) in dichloromethane (14 mL) followed by pyridinium dichromate (0.700 g, 1.86 mmol). The conversion of 47 (Rf 0.54) to the ketone 27 (Rf 0.76) was followed by TLC (1:4 ethyl acetate-hexane). The mixture was diluted with cyclohexane after 4.5 h then filtered trough a layer of silica gel. Filtrate and ether washes were combined and evaporated. The residue was flash-chromatographed (1:39 ethyl acetate-hexane) to give 27 as a colorless syrup, 0.2988 g, 96.6%: ¹H NMR δ: 0.078 (3H, s), 0.097 (3H, s), 0.107 (18H, s), 0.64 (3H, s), 0.81 (1H, m), 0.84 (6H, s), 0.89 (6H,m), 1.134 (3H, s), 1.201 (3H, s), 1.207 (3H, s), 1.211 (3H, s), 1.3-1.6 (14H, m), 1.6-1.7 (3H, m), 1.88 (1H, m), 2.04 (2H, m), 2.2-2.32 (2H, m), 2.46 (1H, dd, J=7.5 and 11.5 Hz), 3.28 (1H, m); LR-FAB(+) m/z: 583 (M−C₆H₁₃), 479 (M−OTMS); HR-ES(+) m/z: Calcd for C₃₇H₇₆O₄Si₃+Na: 691.4943, found: 691.4949.

### [1R,3aR,7aR,4E]-4-{2(Z)-[3(S),5(R)-Bis-(tert-butyl-dimethyl-silanyloxy)-2-methylene-cyclohexylidene]-ethylidene}-7a-methyl-1-[5-methyl-1(R)-(4-methyl-4-trimethylsilanyloxy-pentyl)-4(R)-[dimethyl-(1,1,2-trimethyl-propyl)-silanyloxy]-5-trimethylsilanyloxy-hexyl]-octahydro-indene (49)

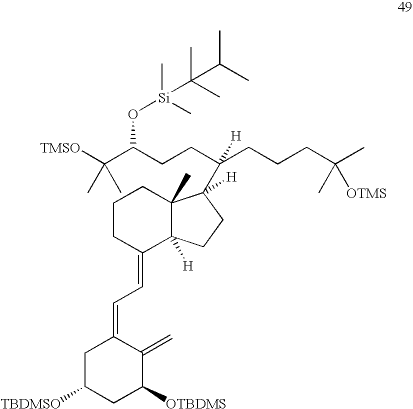

A solution of 2.5-M butyllithium in hexane (0.17 mL) was added to a solution of 28 in tetrahydrofuran (2 mL) at −70° C. to produce a deep cherry-red color of the ylied. After 10 min a solution of ketone 27 (0.1415 g, 0.211 mmol) in tetrahydrofuran (2 mL) was added dropwise over a 15 min period. The reaction was quenched after 4 h by the addition of pH 7 phosphate buffer (2 mL). The temperature was allowed to increase to 0° C. then hexane (30 mL) was added. The aqueous layer was re-extracted with hexane (15 mL). The combined extracts were washed with of brine (5 mL), dried and evaporated to give a colorless oil that was purified by flash-chromatography (1:100 ethyl acetate-hexane) to yield 49 as colorless syrup, 0.155 g, 71%: ¹H NMR δ: 0.068 (15H, m), 0.103 (12H, s), 0.107 (9H, s), 0.53 (3H, s), 0.82 (1H, m), 0.84 (6H, s), 0.88 (18H,m), 0.89 (6H, m), 1.14 (3H, m), 1.20 (9H, s), 12-1.9 (22H, m), 1.97 (2H, m), 2.22 (1H, dd, J=7.5 an 13 Hz), 2.45 (1H, brd, J=13 Hz), 2.83 (1H, brd, J=13 Hz), 3.28 (1H, m), 4.20 (1H, m), 4.38 (1H, m), 4.87 (1H, d, J=2 Hz), 5.18 (1H, d, J=2 Hz), 6.02 (1H, d, J=11.4 Hz, 6.24 (1H, d, J=11.4 Hz); LR-FAB(+) m/z 1033 (M+H), 1032 (M), 1031 (M−H), 901 (M−TBDMS).

### Synthesis of 1,25-Dihydroxy-21-(2R,3-dihydroxy-3-methyl-butyl)-20R-Cholecalciferol (33)

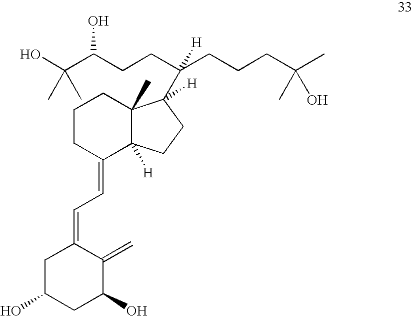

The residue of 49 (0.153 g, 0.148 mmol), as obtained in the previous experiment, was dissolved in a 1 M solution of tetrabutylammonium fluoride (3.5 mL). TLC (ethyl acetate) monitored reaction progress. Thus, the solution was diluted with brine (5 mL) after 24 h, stirred for 5 min then equilibrated with ethyl acetate (35 mL) and water (15 mL). The aqueous layer was re-extracted once with ethyl acetate (15 mL). The combined organic layers were washed with water (5×10 mL), once with brine (5 mL) then dried and evaporated. The residue was purified by flash chromatography using a stepwise gradient of ethyl acetate and 1:100 methanol-ethyl acetate furnishing 33 as colorless, microcrystalline material from methyl formate-pentane, 70 mg, 91%: [α]_{D}+34.3° (methanol, c 0.51); ¹H NMR (DMSO-d₆) δ: 0.051 (3H, s), 0.98 (3H, s), 1.03 (3H, s), 1.05 (6H, s), 1.0-1.6 (17H, m), 1.64 (3H, m), 1.80 (2H, m), 1.90 (1H,d, J=11.7 Hz), 1.97 (1H, dd, J=J=9.8 Hz), 2.16 (1H, dd, J=5.9 and J=13.7 Hz), 2.36 (1H, brd), 2.79 (1H, brd), 3.00 (1H, dd, J=5 and 10 Hz), 3.99 (1H, brs), 4.01 (1H, s, OH), 4.04 (1H, s, OH), 4.54 (1H, OH, d, J=3.9 Hz), 4.76 (1H, brs), 4.87 (1H, OH, d, J=4.9 Hz), 5.22 (1H, brs), 5.99 (1H, d, J=10.7 Hz), 6.19 (1H, d, J=10.7 Hz); LR-ES(+) m/z: 519 (M+H), 518 (M), 517 (M−H), 501 (M−OH); HR-ES(+) calcd for C₃₂H₅₄O₅+Na: 541.3863; found 541.3870; UVₘₐₓ (ε): 213 (13554), 241 sh (12801), 265 (16029) nm.

### Example 42

### Synthesis of 1,25-Dihydroxy-21(2R,3-dihydroxy-3-methyl-butyl)-20S-Cholecalciferol (50)

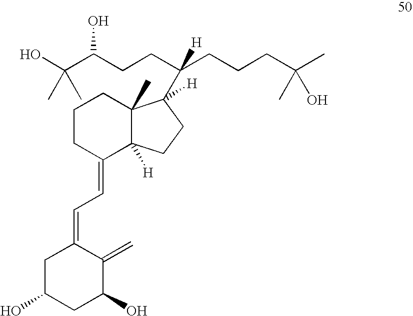

### [1R,3aR,4S,7aR]-7-Benzenesulfonyl-6(R)-[4-(tert-butyl-dimethyl-silanyloxy)-7a-ethyl-octahydro-inden-1-yl]-2-methyl-heptan-2-ol (51)

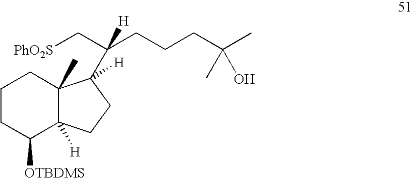

A solution of 36 and sodium benzenesulfinate (0.263 g, 1.6 mmol) in N,N-dimethyl formamide (5 mL) was stirred in a 77° C. bath for 3 h. The solution was equilibrated with 1:1 ethyl acetate-hexane (25 mL) and the organic layer washed with water (5×10 mL), dried and evaporated. The residue was flash-chromatographed with a stepwise gradient of 1:9, 1:4, and 1:3 ethyl acetate-hexane to furnish the sulfone as a colorless syrup: ¹H NMR δ −0.02 (3H, s), 0.005 (3H, s), 0.79 (3H, s), 0.87 (9H, s), 1.12 (1H, m), 1.19 (6H, s), 1.12 (1H, m), 1.20 (6H, s), 1.2-1.8 (18H, m), 2.08 (1H, m), 3.09 (1H, dd, J=9.3 and 14.5 Hz), 3.31 (1H, dd, J=3 and 14.5 Hz), 3.97 (1H, brs), 7.58 (31H, m), 7.66 (1H, m), 7.91 2H, m); LR-ES(+) m/z: 600 (M+Na+MeCN), 559 (M+Na); LR-ES(−) m/z: 536 (M), 535 (M−H); HR-ES(+): Calcd for C₃₀H₅₂O₄SSi+Na 559.3248; found 559.3253.

### [1R,4S,7aR]-1-(1(R)-Benzenesulfonylmethyl-5-methyl-5-trimethylsilanyloxy-hexyl)-4-(tert-butyldimethyl-silanyloxy)-7a-methyl-octahydroindene (52)

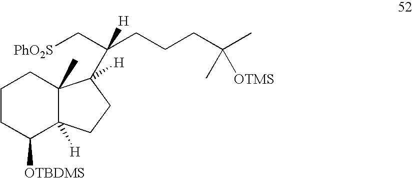

1-(Trimethylsilyl)imidazole (0.146 mL) was added to a solution of 51 (0.145 g, 0.27 mmol) in cyclohexane (2 mL). After 17 h the product was purified by flash chromatography using a stepwise gradient of 1:79 and 1:39 ethyl acetate-hexane to give 52 as colorless residue, 0.157 g 0.258 mmol, TLC (1:9 ethyl acetate-hexane) Rf 0.14. 300 MHz ¹H NMR: δ −0.02 (3H, s), 0.00 (3H, s), 0.87 (12H, s), 1.12 (1H, m), 1.17 (6H, s), 1.2-1.6 (15H, m), 1.6-1.9 (3H, m), 3.08 (2H, m), 3.97 (1H, brs), 7.53-7.70 (3H, m), 7.90 (2H, d, J=7 Hz).

### [1R,3aR,4S,7aR]-5(R,S)-Benzenesulfonyl-6(R)-[4-(tert-butyl-dimethyl-silanyloxy(7a-methyl-octahydro-inden-1-yl]-2,10-dimethyl-10-trimethylsilanyloxy-undecane-2,3(R)-diol (53)

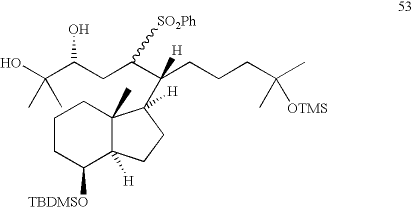

A solution of 152 (0.2589, 0.425 mmol) and diol (0.176 g, 0.638 mmol) in tetrahydrofuran (9 mL) was cooled to −25° C. and 1.6 M butyllithium in hexane (1.4 mL) was added. The temperature was raised to −20° C. and maintained for 3 h then at −10° C. for 2.5 h and 0° C. for 10 min. The mixture was cooled again to −10° C., saturated ammonium chloride solution (5 mL) was added, then equilibrated with ethyl acetate (50 mL) and enough water to dissolve precipitated salts. The aqueous layer was re-extracted with ethyl acetate (15 mL), the combined extracts were dried and evaporated and the residue purified by flash chromatography using a stepwise gradient of 1:6, 1:4, and 1:1 ethyl acetate-hexane to produce 53 as a colorless syrup, 0.212 g, 70%: 300 MHz ¹H NMR: δ 0.00 (3H, s), 0.017 (3H, s), 0.12 (9H, s), 0.81 (3H, s), 0.89 (9H, s), 1.16 (1H, m), 1.19 (12H, m), 1.1-1.6 (20H, m), 1.6-1.8 (2H, m), 3.10 (1H, dd, J=8.4 and 14.7 Hz), 3.30 (1H, m), 3.99 (1H, brs), 7.61 (2H, m), 7.67 (1H, m), 7.93 (2H, m).

### [1R,3aR,4S,7aR]-6(S)-[4-(tert-Butyl-dimethyl-silanyloxy)-7a-methyl-octahydro-inden-1-yl]-2,10-dimethyl-10-trimethylsilanyloxy-undecane-2,3(R)-diol (54)

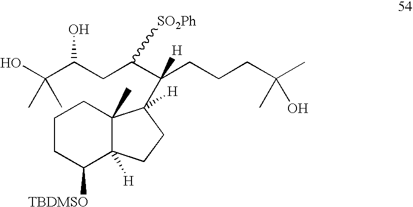

Compound 53 (0.186 mg, 0.262 mmol) was dissolved in 0.5 M oxalic acid dihydrate in methanol (2.5 mL). The solution was stirred for 15 min then calcium carbonate was added (0.5 g) and the suspension stirred overnight then filtered. The filtrate was evaporated to give 54 as a white foam, 0.188 g, 98%: TLC (1:1 ethyl acetate-hexane) Rf 0.06. This material was used in the next step without further purification.

### [1R,3aR,4S,7aR]-6(S[4-(tert-Butyl-dimethyl-silanyloxy)-7a-methyl-octahydro-inden-1-yl]-2,10-dimethyl-undecane-2,3(R),10-triol (triol 55)

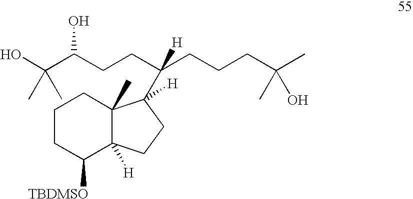

Sodium amalgam (5% sodium, 10.8 g) was added to a vigorously stirred solution of 54 (0.426 g, 0.667 mmol) in a mixture of tetrahydrofuran (15 mL) and methanol (9 mL). The suspension was stirred for 24 h and the reaction monitored by TLC (1:1 ethyl acetate-hexane0 to observe the production of 55 (Rf 0.17). The mixture was diluted with methanol (3 mL), stirred for 5 min then further diluted with water (10 mL), stirred for 2 min and decanted into saturated ammonium chloride solution (25 mL). The aqueous layer was extracted with ethyl acetate (2×20 mL). The combined extracts were washed with pH 7 phosphate buffer (5 mL) then brine (10 mL), dried and evaporated. The residue was purified by flash-chromatography using a stepwise gradient of 1:1 and 2:1 ethyl acetate-hexane to provide 55 as a colorless syrup, 0.244 g, 73%: ¹H NMR: δ−0.006 (3H, s), 0.006 (3H, s), 0.86 (9H, s), 0.92 (3H, s), 1.11 (1H, m), 1.15 (3H, s), 1.21 (9H, s), 1.2-1.75 (21H, m), 1.7-1.85 (3H, m), 1.90 (1H, m), 3.29 (1H, brd), 3.99 (1H, brs); LR-ES(+) m/z: 521 (M+Na), 481 (M−OH); LR-ES(−): m/z 544: (M+CH₂O₂), 543 (M−H+ CH₂O₂), 533 (M−Cl); HR-ES(+) m/z: Calcd for C₂₉H₅₈O₄Si+Na: 521.3996, found 521.3999.

### [1R,3aR,4S,7aR]-6(S)-(4-Hydroxy-7a-methyl-octahydro-inden-1-yl)-2,10-dimethyl-undecane-2,3(R),10-triol (56)

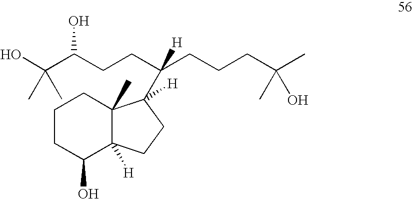

An aqueous fluorosilicic acid solution (3 mL) was added to a stirred solution of 55 (0.240 g, 0.481 mmol) in acetonitrile (12 mL). TLC (ethyl acetate) monitored the reaction. After 2.5 h compound 56 (Rf 0.37) was the predominating species, produced at the expense of less polar 55. The mixture was equilibrated with ethyl acetate and water (10 mL), the aqueous layer was re-extracted with water (2×10 mL) and the combined extracts were washed with water (6 mL) and brine (2×10 mL) then dried and evaporated. The colorless residue was flash-chromatographed using a stepwise gradient of 1:2, 1:1 and 2:1 ethyl acetate-hexane to elute some unreacted 55, followed by 56, obtained as colorless syrup, 0.147 g, 79%: ¹H NMR: 0.94 (3H, s), 1.12 (1H, m), 1.15 (3H, s), 1.21 (9H, s), 1.15-1.7 (20H, m), 1.7-1.9 (5H, m), 1.96 (1H, brd), 3.29 (1H, d, J=9.6 Hz), 4.08 (1H, brs); LR-ES(+): m/z 448: (M+Na+MeCN), 407 (M+Na); LR-ES(−): m/z 419 (M+Cl); HR-ES(+) m/z: Calcd for C₂₃E₄₄O₄+Na: 407.3132, found 407.3135.

### [1R,3aR,4S,7aR]-1-(5-Hydroxy-1(S)-{2-[2-(4-methoxy-phenyl-5,5-dimethyl-[1,3]dioxolan-4(R)-yl]-ethyl}-5-methyl-hexyl)-7a-methyl-octahydro-inden-4-ol (57)

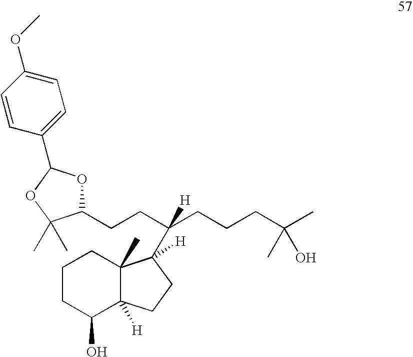

4-Methoxybenzaldehyde dimethyl acetal (60 μL, 0.35 mmol) was added to a solution of 56 (81.2 mg, 0.211 mmol) in dichloromethane (2 mL), followed by a solution (0.2 mL) containing pyridinium tosylate (200 mg) in dichloromethane (10 mL). Reaction progress was followed by TLC (1:2 ethyl acetate-hexane) which showed 4-methoxybenzaldehyde dimethyl acetal (Rf 0.80), 4-methoxybenzaldehyde (Rf 0.65), educt 56 (Rf 0.42) and product 57 (Rf 0.26). After 5¾ h the mixture was stirred for 15 min with saturated sodium hydrogencarbonate solution (5 mL) then equilibrated with ethyl acetate (25 mL). The organic layer was washed with brine (5 mL), dried and evaporated. The residue was flash-chromatographed using a stepwise gradient of 1:3 and 1:2 ethyl acetate-hexane to yield 57 as colorless syrup, 0.106 mg (100%): ¹H NMR: 0.94 (3H, s), 1.19, 1.21 (6H, s each, Me₂COH), 1.23, 1.35 and 1.24, 1.37 (6H, s each, major and minor 5,5-dimethyloxolane diastereomer), 1.1-1.7 (18H, m), 1.7-1.9 (5H, m), 1.9-2.0 (2H, m), 3.65 (1H, m), 3.81 (3H, s), 4.08 (1H, brs), 5.78 and 5.96 (1H, s each, major and minor acetal diastereomer), 6.89 (2H, m), 7.41 (2H, m).

### [1R,3aR,7aR]-1-(5-Hydroxy-[(S)-{2-[2-(4-methoxy-phenyl-5,5-dimethyl-[1,3]dioxolan-4(R)-yl]-ethyl}-5-methyl-hexyl)-7a-methyl-octahydro-inden-4-one (58)

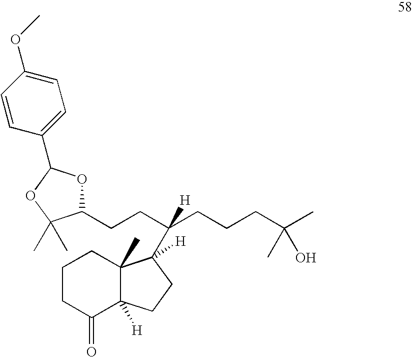

Pyridinium dichromate (230 mg, 0.61 mmol) was added to a stirred mixture containing 57 (0.0838, 0.167 mmol), Celite (185 mg), and dichloromethane (4 mL). The conversion of 57 (Rf 0.31) to 58 (Rf 0.42) was monitored by TLC (1:25 methanol-chloroform) The mixture was diluted with dichloromethane (10 mL) after 2.5 h, then filtered through a layer of silica gel. Filtrate and washings (1:1 dichloromethane-ethyl acetate) were evaporated and the residue chromatographed (1:4 ethyl acetate-hexane) to give ketone 58, 0.0763 g, 91%: ¹H NMR: 0.63 (3H, s), 1.19, 1.21 and 1.23 (6H, s each, Me₂COH), 1.25, 1.36, 1.38 (6H, m,s,s, 5,5-dimethyloxolane diastereomer), 1.1-1.9 (18H, m), 1.9-2.1 (3H, m), 2.1-2.4 (2H, m), 2.45 (1H, m), 3.66 (1H, m), 3.802 and 3.805 (3H, s each), 5.78 and 5.95 (1H, s each, major and minor acetal diastereomer), 6.89 (2H, m), 7.39 (2H, m).

### [1R,3aR,7aR]-1-[4(R),5-Dihydroxy-1(S)-(4-hydroxy-4-methyl-pentyl)-5-methyl-hexyl]-7a-methyl-octahydro-inden-4-one (59)

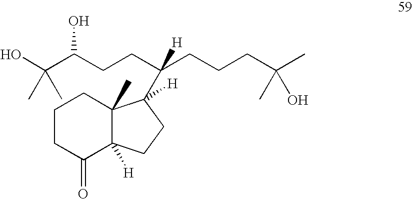

The ketone 58 was stirred in a 1 N oxalic acid solution in 90% methanol. The mixture became homogeneous after a few min. TLC (ethyl acetate) suggested complete reaction after 75 min (Rf 0.24 for 59). Thus, calcium carbonate (0.60 g) was added and the suspension stirred overnight, then filtered. The filtrate was evaporated and flash-chromatographed using a stepwise gradient of 4:1:5 dichloromethane-ethyl acetate-hexane, 1:1 ethyl acetate-hexane, and neat ethyl acetate produce 59 as a colorless residue, 0.060 mg, 94%: ¹H NMR: 0.5 (3H, s), 1.17 (3H, s), 1.22 (6H, s), 1.23 (3H, s), 1.2-1.21 (23H, m), 2.15-2.35 (2H, m), 2.45 (1H, dd, J=7 and 11 Hz), 3.30, 1H, brd).

### [1R,3aR,7aR]-7a-Methyl-1-[5-methyl-1(S)-(4-methyl-4-triethylsilanyloxy-pentyl)-4(R),5-bis-triethylsilanyloxy-hexyl]-octahydro-inden-4-one (60)

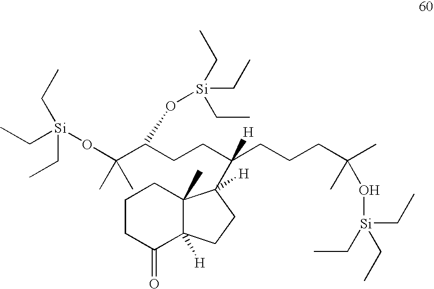

A mixture of 59 (0.055 g, 0.143 mmol), imidazole, (14.9 mg, 1.69 mmol), N,N-dimethylpyridine (6 mg), triethylchlorosilane (0.168 mL, 1 mmol) and N,N-dimethylformamide (1.5 mL) was stirred for 17 h. The reaction was followed by TLC (1:4 ethyl acetate-hexane) and showed rapid conversion to the disilyl intermediate (Rf 0.47). Further reaction proceeded smoothly overnight to give the fully silylated 60 (Rf 0.90). The solution was equilibrated with water (3 mL), equilibrated with ethyl acetate (20 mL), the ethyl acetate layer was washed with water (3×4 mL), dried and evaporated. The residue was flash-chromatographed using a stepwise gradient of hexane and 1:100 ethyl acetate-hexane to yield 60 as a colorless syrup, 0.0813 g, 78.4%: ¹H NMR δ 0.55-0.64 (21H, m), 0.92-0.97 (27H, m), 1.12 (3H, s), 1.18 (3H, s), 1.19 (3H, s), 1.21 (3H, s), 1.1-1.7 (18H, m), 1.9-2.15 (2H, m), 2.15-2.35 (2H, m), 2.43 (1H, dd, J=7.7 and 11 Hz), 3.30 (1H, dd, J=3 and 8.4 Hz).

### [1R,3aR,7aR,4E]-4-{2(Z)-[3(S),5(R)-Bis-(tert-butyl-dimethyl-silanyloxy)-2-methylene-cyclohexylidene]-ethylidene}-7a-methyl-1-[5-methyl-1(S)-(4-methyl-4-triethylsilanyloxy-pentyl)-4(R),5-bis-triethylsilanyloxy-hexyl]-octahydro-indene 61)

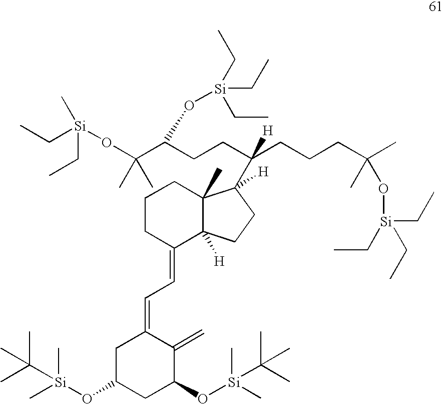

A solution of 1.6 M butyllithium in hexane (0.14 mL) was added to a solution of phosphine (0.1308 g, 0.224 mmol) in tetrahydrofuran (1.5 mL) at −70° C. After 10 min a solution of ketone 60 (0.0813 g, 0.112 mmol) in tetrahydrofuran (1.5 mL) was added dropwise over a 15 min period. The ylide color had faded after 3 h so that pH 7 phosphate buffer (2 mL) was added and the temperature allowed to increase to 0° C. The mixture was equilibrated with hexane (30 mL), the organic layer was washed with brine (5 mL), dried and evaporated to give a colorless oil that was purified by flash-chromatography (1:100 ethyl acetate-hexane). Only the band with Rf 0.33 (TLC 1:39 ethyl acetate-hexane) was collected. Evaporation of those fractions gave 61 as colorless syrup, 0.070 g, 57%: ¹H NMR δ 0.06 (12H, brs), 0.53-0.64 (21H, m), 0.88 (18H, s), 0.92-0.97 (27H, m), 1.11 (3H, s), 1.177 (3H, s), 1.184 (3H, s), 1.195 (3H, s), 1-1.9 (22H, m), 1.98 (2H, m), 2.22 (1H, m), 2.45 (1H, m), 2.83 (1H, brd, J=13 Hz, 3.27 (1H, d, J=6 Hz), 4.19 (1H, m), 4.38 (1H, m), 4.87 (1H, brs), 5.18 (1H, brs), 6.02 (1H, d, J=11 Hz), 6.24 (1H, d, J=11 Hz).

### Synthesis of 1,25-Dihydroxy-21(2R,3-dihydroxy-3-methyl-butyl)-20S-Cholecalciferol (50)

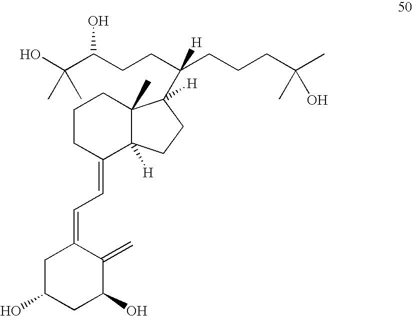

The deprotection reaction of 61 (0.068 g, 0.06238 mmol) in 1M solution of tetrabutylammonium fluoride in tetrahydrofuran, followed by TLC (ethyl acetate), gradually proceeded to give 50 (Rf 0.19). The mixture was diluted with brine (5 mL) after 25 h, stirred for 5 min the equilibrated with ethyl acetate (35 mL) and water (15 mL). The aqueous layer was re-extracted once with ethyl acetate (35 mL), the combined extracts were washed with water (5×10 mL) and brine (5 mL) then dried and evaporated. The residue was flash-chromatographed using a linear gradient of 1:1 and 2:1 ethyl acetate-hexane, and 2:98 methanol-ethyl acetate to give a residue that was taken up in methyl formate and evaporated to a white foam, 30 mg, 93%: [α]_{D}+29.3° (methanol, c 0.34); MHz ¹H NMR δ: 0.55 (3H, s), 1.16 (3H, s), 1.21 (9H, s), 1.1-1.75 (22H, m), 1.80 (2H, m), 1.9-2.1 (5H, m), 2.31 (1H, dd, J=7 and 13 Hz), 2.60 (1H, brd), 284 (1H, m), 3.29 (1H, d, J=9.5 Hz), 4.22 (1H, m), 4.43 (1H, m), 5.00 (1H, s), 5.33 (1H, s), 6.02 (1H, d, J=11 Hz), 6.02 (1H, d, J=11 Hz); LR-ES(−) m/z: 564 (M+H2CO2), 563 M-H+ H2CO2); HR-ES(+) calcd for C₃₂H₅₄O₅+Na: 541.3863; found 541.3854; UVₘₐₓ (ε): 211 (15017), 265 (15850), 204 sh (14127), 245 sh (13747) nm.

### Example 43

### Synthesis of 1,25-Dihydroxy-21-(2R,3-dihydroxy-3-methyl-butyl)-20S-19-nor-cholecalciferol (62)

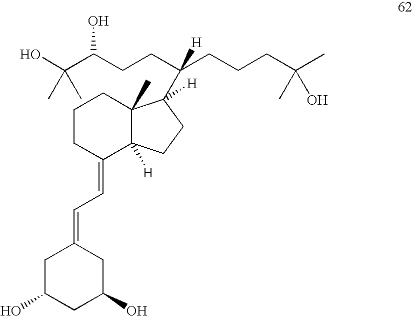

### [1R,3aR,7aR,4E]-4-{2(Z)-[3(S),5(R)-Bis-(tert-butyl-dimethyl-silanyloxy)-cyclohexylidene]-ethylidene}-7a-methyl-1-[5-methyl-1(S)-(4-methyl-4-triethylsilanyloxy-pentyl)-4(R),5-bis-triethylsilanyloxy-hexyl]-octahydro-indene (63)

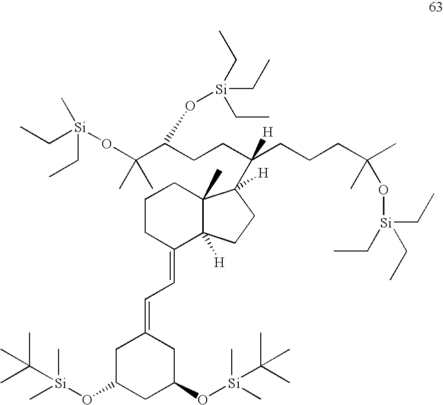

A solution of 1.6 M butyllithium in hexane was added to a solution of phosphine in tetrahydrofuran at −70° C. After 10 min a solution of ketone 60 from Example 2 in tetrahydrofuran was added dropwise over a 15 min period. After the ylide color had faded, pH 7 phosphate buffer was added and the temperature allowed to increase to 0° C. The mixture was equilibrated with hexane, the organic layer was washed with brine, dried and evaporated to give a colorless oil that was purified by flash-chromatography (1:100 ethyl acetate-hexane) that gave 63.

### 1,25-Dihydroxy-21-(2R,3-dihydroxy-3-methyl-butyl)-20S-19-nor-cholecalciferol (62)

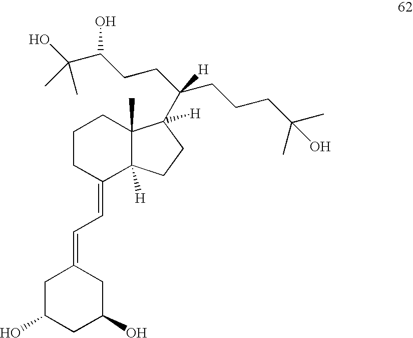

The deprotection reaction of 63 was carried out in 1M solution of tetrabutylammonium fluoride in tetrahydrofuran to give 62. The mixture was diluted with brine after 25 h, stirred for 5 min and then equilibrated with ethyl acetate and water. The aqueous layer was re-extracted once with ethyl acetate, the combined extracts were washed with water and brine, and then dried and evaporated. The residue was flash-chromatographed to give a residue that was taken up in methyl formate and evaporated to yield 62.

### Example 44

### Synthesis of 1,25-dihydroxy-20S-21(3-hydroxy-3-methyl-butyl)-24-keto-19-nor-cholecalciferol (64)

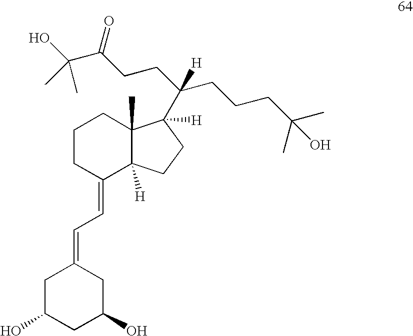

### (R)-6-[(1R,3aR,4S,7aR)-4-(tert-Butyl-dimethyl-silanyloxy)-7a-methyl-octahydro inden-1-yl]-2-methyl-7-phenylsulfanyl-heptan-2-ol (65)

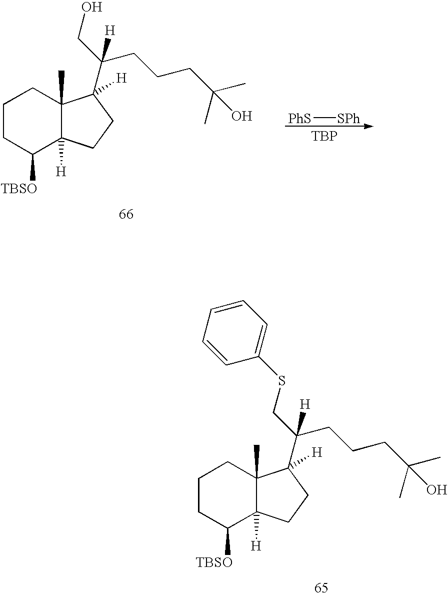

The reaction above was carried out as described in Tet. Lett. 1975, 17: 1409-12. Specifically, a 50 mL round-bottom flask was charged with 1.54 g (3.73 mmol) of (R)-2-[(1R,3aR,4S,7aR)-4-(tert-Butyldimethylsilanyloxy)-7a-methyloctahydroinden-1-yl]-6-methylheptane-1,6-diol (1) (Eur. J. Org. Chem. 2004, 1703-1713) and 2.45 g (11.2 mmol) of diphenylsulfide. The mixture was dissolved in 5 mL of pyridine and 2.27 g (11.2 mmol, 2.80 mL) of tributylphosphine was added. The mixture was stirred overnight and then diluted with 20 mL of toluene and evaporated. The residue was again taken up in toluene and evaporated, the remaining liquid chromatographed on silica gel using stepwise gradients of hexane, 1:39, 1:19 and 1:9 ethyl acetate-hexane to provide the title compound 65 as a syrup, 1.95 g.

### (R)-7-Benzenesulfonyl-6-[(1R,3aR,4S,7aR)-4-(tert-butyl-dimethyl-silanyloxy)-7a-methyl-octahydro-inden-1-yl]-2-methyl-heptan-2-ol (67) and (1R,3aR,4S,7aR)-1-((R)-1-Benzenesulfonylmethyl-5-methyl-5-triethylsilanyloxy-hexyl)-4-(tert-butyl-dimethyl-silanyloxy)-7a-methyl-octahydro-indene (68)

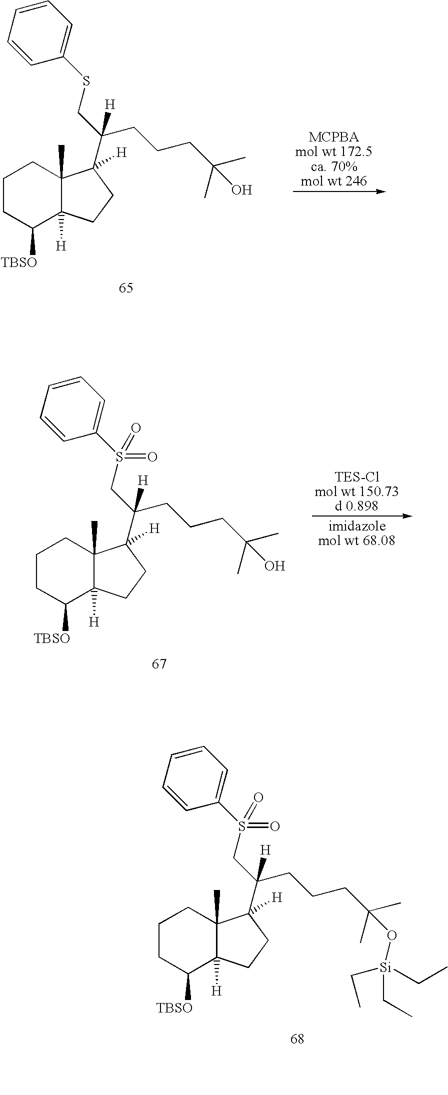

A 500-mL round-bottom flask containing 1.95 g (3.9 mmol) of the crude sulfide 65 was admixed with 84 g of dichloromethane (63 mL). The solution was stirred in an ice bath, then 2.77 g (11 mmol) of meta-chloroperbenzoic acid was added in one portion. The suspension was stirred in the ice bath for 40 min then at room temperature for 2 h. The reaction was monitored by TLC (1:19 methanol-dichloromethane). At the end of the reaction period, only one spot at Rf 0.45 observed. Then, 1.68 g (20 mmol) of solid sodium hydrogen carbonate was added to the suspension, the suspension was stirred for 10 min, then 30 mL of water was added in portions and vigorous stirring continued for 5 min to dissolve all solids. The mixture was further diluted with 40 mL of hexane, stirred for 30 min, transferred to a separatory funnel with 41.6 g of hexane. The lower layer was discarded and the upper one was washed with 25 mL of saturated sodium hydrogen carbonate solution, dried (sodium sulfate) and evaporated to give 3.48 g of 67. This material was triturated with hexane, filtered, and evaporated, to leave 67 as a cloudy syrup (2.81 g) that was used directly in the next step.

A 100-mL round bottom flask containing 2.81 g of 67 obtained above, was charged with 30 mL of N,N-dimethylformamide 1.43 g of (21 mmol) of imidazole and 1.75 mL of (10 mmol) of triethylsilyl chloride. The mixture was stirred for 17 h then diluted with 50 g of ice-water, stirred for 10 min, further diluted with 5 mL of brine and 60 mL of hexane. The aqueous layer was re-extracted with 20 mL of hexane, both extracts were combined, washed with 2×30 mL of water, dried, evaporated. This material contained a major spot with Rf 0.12 (1:39 ethyl acetate-hexane) and a minor spot with Rf 0.06. This material was chromatographed on silica gel using hexane, 1:100, 1:79, 1:39 and 1:19 ethyl acetate-hexane as stepwise gradients. The major band was eluted with 1:39 and 1:19 ethyl acetate-hexane to yield 1.83 g of 68.

### (R)-5-Benzenesulfonyl-6-[(1R,3R,4S,7aR)-4-(tert-butyl-dimethyl-silanyloxy)-7a-methyl-octahydro-inden-1-yl]-10-methyl-2-(R)-methyl-10-triethylsilanyloxy-undecane-2,3-diol (69)

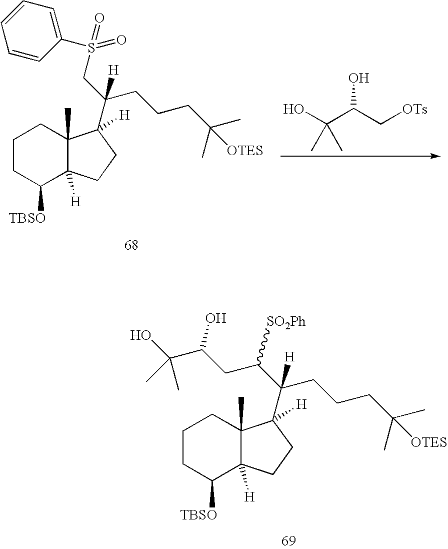

A 100-mL 3-neck round-bottom flask, equipped with magnetic stirrer, thermometer and Claisen adapter with rubber septum and nitrogen sweep, was charged with 1.7636 g of (2.708 mmol) of sulfone 68, 1.114 g of (4.062 mmol) tosylate, and 50 mL of tetrahydrofuran freshly distilled from benzophenone ketyl. This solution was cooled to −20° C. and 9.31 mL of a 1.6 M butyllithium solution in hexane was added dropwise at ≦−20° C. The temperature range between −10 and −20° C. was maintained for 5 h. The cooling bath was removed and 50 mL of saturated ammonium chloride solution added followed by 75 mL of ethyl acetate and enough water to dissolve all salts. The organic layer was washed with 15 mL of brine, dried, and evaporated to a colorless oil. This residue was chromatographed on silica gel using hexane, 1:9, 1:6, 1:4 and 1:3 ethyl acetate-hexane as stepwise gradients. The main band was eluted with 1:4 and 1:3 ethyl acetate-hexane to furnish 1.6872 g of compound 69 as colorless syrup.

### (S)-6-[(1R,3aR,4S,7aR)-4-(tert-Butyl-dimethyl-silanyloxy)-7a-methyl-octahydro-inden-1-yl]-10-methyl-2-(R-methyl-10-triethylsilanyloxy-undecane-2,3-diol (70)

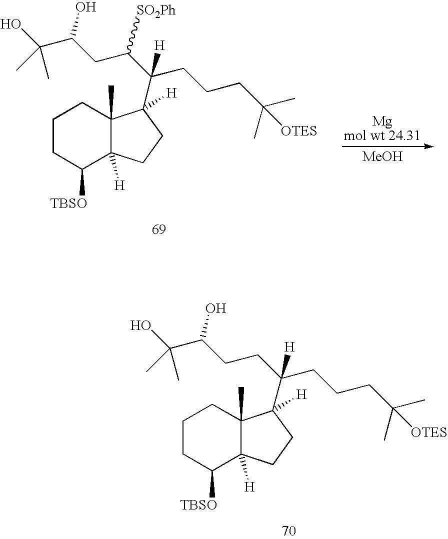

A 25-mL 2-neck round-bottom flask, equipped with magnetic stirrer, thermometer and Claisen adapter with rubber septum and nitrogen sweep, was charged with 1.6872 g (2.238 mmol) of sulfone 69 and 40 mL of methanol. Then 1.25 g (51.4 mmol) of magnesium was added to the stirred solution in two equal portions, in a 30 min time interval. The suspension was stirred for 70 min then another 0.17 g of magnesium and ca. 5 mL of methanol was added and stirring continued 1 h. The mixture was then diluted with 100 mL of hexane and 50 mL of 1 M sulfuric acid was added dropwise to give two liquid phases. The aqueous layer was neutral. The aqueous layer was re-extracted once with 25 mL of 1:1 dichloromethane-hexane. The organic layers were combined then washed once with 15 mL of brine, dried and evaporated. The resulting material was chromatographed on silica gel using hexane, 1:39, 1:19 and 1:9 ethyl acetate-hexane as stepwise gradients. The main band was eluted with 1:9 ethyl acetate-hexane to provide 1.2611 g of 70 as a colorless syrup.

### (S)-6-[1R,3aR,4S,7aR-4-(tert-Butyl-dimethyl-silanyloxy)-7a-methyl-octahydro-inden-1-yl]-2,10-dihydroxy-2,10-dimethyl-undecan-3-one (71)

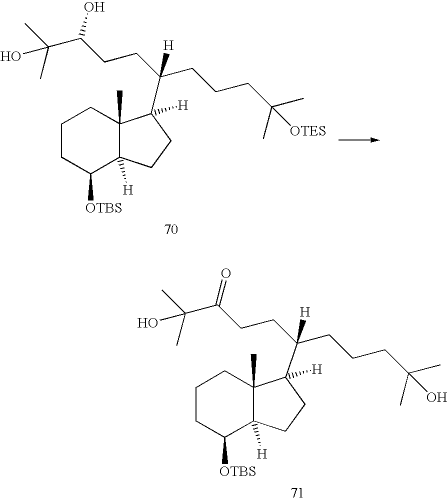

A 25-mL round-bottom flask, equipped with magnetic stirrer, thermometer, Claisen adapter with nitrogen sweep and rubber septum, was charged with 518 mg (3.88 mmol) of N-chlorosuccinamide and 11 mL of toluene. Stir for 5 min (not all dissolved), then cool to 0° C. and add 2.4 mL (4.8 mmol) of a 2M dimethyl sulfide solution in toluene. The mixture was stirred from 5 min then cooled to −30° C. and a solution of 0.7143 g (1.165 mmol) of the diol 70 in 4×1.5 mL of toluene was added dropwise at −30° C. Stirring was continued at this temperature for 1 h. The mixture was then allowed to warm to −10° C. during a 2 h time period then cooled to −17° C. and 3.20 mL (6.4 mmol) of 2 M triethylamine in toluene added dropwise. The mixture was stirred at −17 to −20° C. for 10 min then allowed to warm to room temperature slowly. The mixture was chromatographed on a silica gel column using hexane, 1:79, 1:39, 1:19, 1:9, 1:4, and 1:1 ethyl acetate-hexane as stepwise gradients. The major band was eluted with 1:1 ethyl acetate-hexane providing 0.3428 g of the compound 71 as solids.

### (S)-2,10-Dihydroxy-6-((1R,3aR,4S,7aR)-4-hydroxy-7a-methyl-octahydro-inden-1-yl)-2,10-dimethyl-undecan-3-one (72)

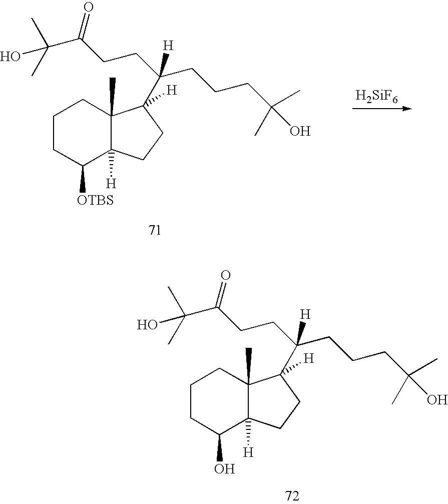

A 25-mL round-bottom flask, equipped with magnetic stirrer was charged with 0.3428 g (0.69 mmol) of the diol 71, was dissolved in 5 mL of acetonitrile then 1.25 mL of fluorosilicic acid solution. After 3 h, the mixture was distributed between 35 mL of ethyl acetate and 10 mL of water, the aqueous layer was re-extracted with 10 mL of ethyl acetate, the organic layers combined, washed with 2×5 mL of water, once with 5 mL of 1:1 brine-saturated sodium hydrogen carbonate solution, dried and evaporated. This material was chromatographed on silica gel using 1:4, 1:3, 1:2, and 1:1 as stepwise gradients furnishing 0.2085 g of the title compound 72.

### (1R,3aR,7aR)-1-[(S)-5Hydroxy-1-(4-hydroxy-4-methyl-pentyl)₅-methyl-4-oxo-hexyl]-7a-methyl-octahydro-inden-4-one (73)

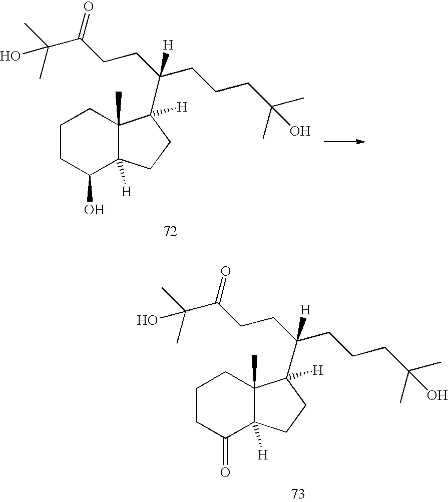

A 25-mL round bottom flask was charged with 0.2153 g (0.56 mmol) of 72, 5 mL of dichloromethane, and 0.20 g of Celite. To this stirred suspension was added, in on portion, 1.00 g (2.66 mmol) of pyridinium dichromate. The reaction stirred for 3 h and the progress was monitored by TLC (1:1 ethyl acetate-hexane). The reaction mixture was diluted with 5 mL of cyclohexane then filtered trough silica gel G. The column was eluted with dichloromethane followed by 1:1 ethyl acetate-hexane until no solute was detectable in the effluent. The effluent was evaporated and the colorless oil. This oil was then chromatographed on a silica gel using 1:4, 1:3, 1:2, 1:1 and 2:1 ethyl acetate-hexane as stepwise gradients to furnish 0.2077 g of the diketone 73.

### (1R,3aR,7)-7a-Methyl-1-[(S)-5-methyl-1-(4-methyl-4-trimethylsilanyloxy-pentyl)-4-oxo-5-trimethylsilanyloxy-hexyl]-octahydro-inden-4-one (74)

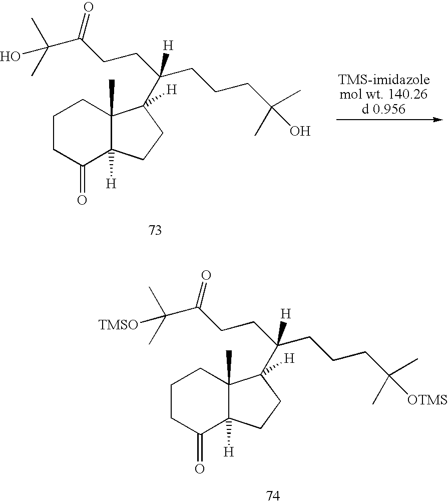

A 25-mL round bottom flask was charged with 0.2077 g (0.545 mmol) of the diketone 73. This material was dissolved in a mixture of 0.5 mL of tetrahydrofuran and 3 mL of cyclohexane. To the resulting mixture was added 0.30 mL (2.0 mmol) Of TMS-imidazole. The reaction mixture was diluted with 3 mL of hexane after 10 h then concentrated and chromatographed on silica gel using hexane, 1:79, 1:39, 1:19 and ethyl acetate-hexane as stepwise gradients to provide 0.2381 g of 74 as a colorless oil.

### (S)-6-((1R,3aS,7aR)-4-{2-[(R)-3-((R)-tert-Butyldimethylsilanyloxy)₅-(tert-butyidimethylsilanyloxy)-cyclohexylidene]-ethylidene}-7a-methyloctahydroinden-1-yl)-2,10-dimethyl-2,10-bis-trimethylsilanyloxyundecan-3-one (75)

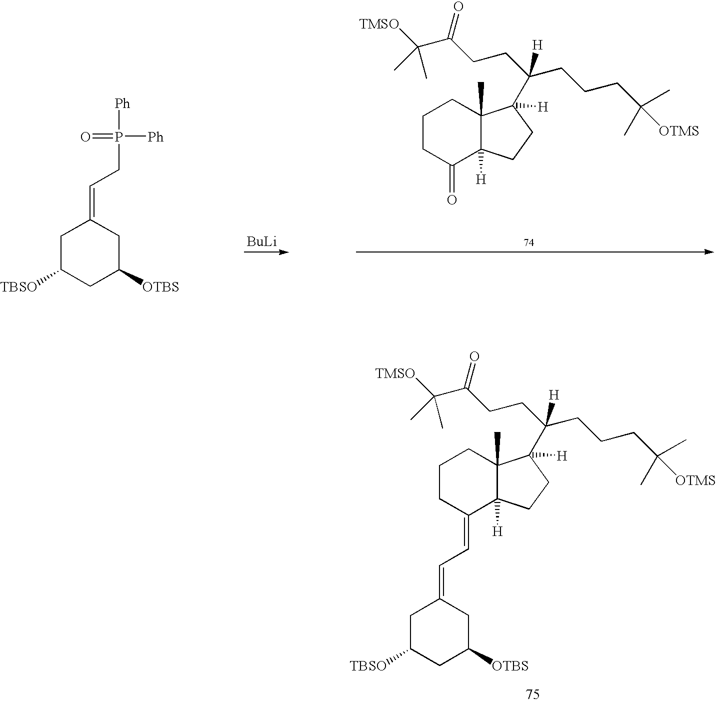

A 15-mL 3-neck pear-shaped flask, equipped with magnetic stirrer, thermometer and a Claisen adapter containing a nitrogen sweep and rubber septum, was charged with 0.2722 g (0.4768 mmol) of [2-[(3R,5R)-3,5-bis(tert-butyldimethylsilanyloxy) cyclohexylidene]ethyl]diphenylphosphine oxide and 2 mL of tetrahydrofuran. The solution was cooled to −70° C. and 0.30 mL of 1.6 M butyllithium in hexane was added. The deep red solution was stirred at that temperature for 10 min then 0.1261 g (0.240 mmol) of the diketone 74, dissolved in 2 mL of tetrahydrofuran was added, via syringe, dropwise over a 10 min period. After 3 h and 15 min, 5 mL of saturated ammonium chloride solution was added at −65° C., the mixture allowed to warm to 10° C. then distributed between 35 mL of hexane and 10 mL of water. The aqueous layer was re-extracted once with 10 mL of hexane, the combined layers washed with 5 ml of brine containing 2 mL of pH 7 buffer, then dried and evaporated. This material was chromatographed on a flash column, 15×150 mm using hexane and 1:100 ethyl acetate-hexane as stepwise gradients to yield 0.1572 g of the title compound 75 as a colorless syrup.

### 1,25-Dihydroxy-20S-21(3-hydroxy-3-methyl-butyl)-24-keto-19-nor-cholecalciferol (64)

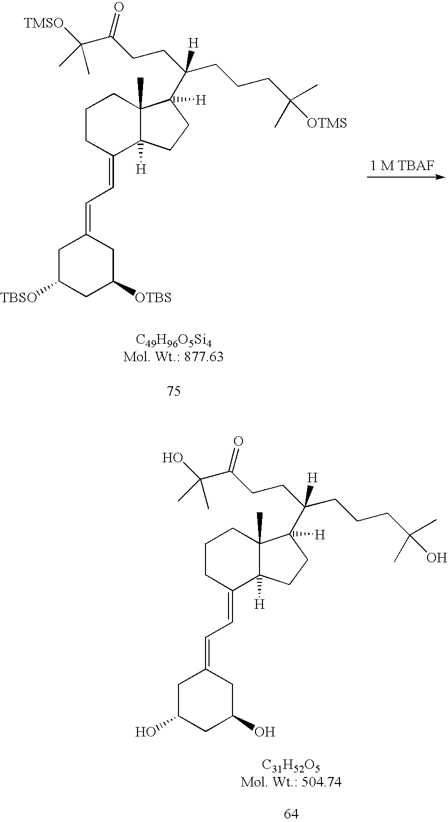

A 15-mL 3-neck round-bottom flask, equipped with magnetic stirrer, was charged with 155 mg (0.17 mmol) of tetrasilyl ether 75. This colorless residue was dissolved is 2 mL of a 1 M solution of tetrabutylammonium fluoride in tetrahydrofuran. After 43 h an additional 0.5 mL of 1 M solution of tetrabutylammonium fluoride solution was added and stirring continued for 5 h. The light-tan solution was the diluted with 5 mL of brine, stirred for 5 min and transferred to a separatory funnel with 50 mL of ethyl acetate and 5 mL of water then re-extraction with 5 mL of ethyl acetate. The organic layers were combined, washed with 5×10 mL of water, 10 mL of brine, dried and evaporated. The resulting residue was chromatographed on a 15×123 mm column using 2:3, 1:1, 2:1 ethyl acetate-hexane, and ethyl acetate as stepwise gradients to provide the 64 as a white solid (TLC, ethyl acetate, Rf 0.23) that was taken up in methyl formate, filtered and evaporated furnishing 0.0753 g of the title compound 64 as a solid substance.

### Example 45

### Synthesis of 1,25-dihydroxy-20S-21(3-hydroxy-3-methyl-butyl)-24-keto-cholecalciferol (76)

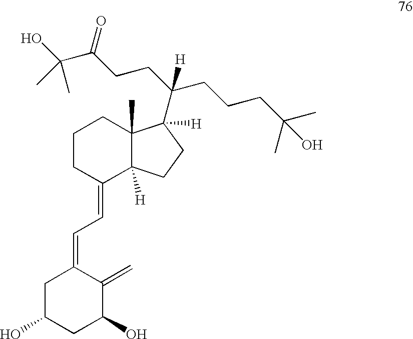

### (S)-6-{(1R,3aS,7aR)-4-[2-[(R)-3-(tert-Butyl-dimethyl-silanyloxy)-5-((S)-tert-butyl-dimethyl-silanyloxy)-2-methylene-cyclohexylidene]-eth-(E)-ylidene]-7a-methyl-octahydro-inden-1-yl}-2,10-dimethyl-2,10-bis-trimethylsilanyloxy-undecan-3-one (77)

Compound 77 was prepared as described for 75 in Example 4 but by reacting 74 with [(2Z)-2-[(3S,5R)-3,5-bis(tert-butyldimethylsilanyloxy) methylenecyclohexylidene]-ethyl]diphenylphosphine oxide.

### 1,25-Dihydroxy-20S-21(3-hydroxy-3-methyl-butyl)-24-keto-cholecalciferol (76)

Compound 76 was prepared from 77 by deprotecting 77 as described in Example 22 for 64.

### Example 46

### Synthesis of 1,3-O-Diacetyl-1,25-Dihydroxy-16-ene-24-Keto-19-nor-Cholecalciferol (78)

Referring to Scheme 1 below, compounds of formula I of the invention are prepared as shown in Scheme 1 below. Accordingly, compounds of formula I (wherein X₁ and X₂ are each independently H₂ or ═CH₂, provided X₁ and X₂ are not both ═CH₂; R₁ and R₂ are each independently, hydroxyl, OC(O)C₁-C₄ alkyl, OC(O)hydroxyalkyl or OC(O)fluoroalkyl, provided that R₁ and R₂ are not both hydroxyl; R₃ and R₄ are each independently hydrogen, C₁-C₄ alkyl, or R₃ and R₄ taken together with C₂₀ form C₃-C₆ cycloalkyl; R₅ and R₆ are each independently C₁-C₄ alkyl, hydroxyalkyl, or haloalkyl, e.g., fluoroalkyl, e.g., fluoromethyl and trifluoromethyl) are prepared by coupling compounds of formula II with compounds of formula III in tetrahydrofuran with n-butyllithium as a base to give compounds of formula IV. Subsequent removal of the protecting silyl groups (R₁=OSi(CH₃)₂t.Bu) affords the 1,3 dihydroxy vitamin D₃ compound of formula I (R₁=OH, R₂═OH). Acylation at the 1 and/or 3 positions is achieved using methods well-known in the art. For example, preparation of the 1,3 diacetoxy compounds of formula I (R₁═R₂=OAc) requires additional acetylation with acetic anhydride and pyridine, as shown in Scheme 2. 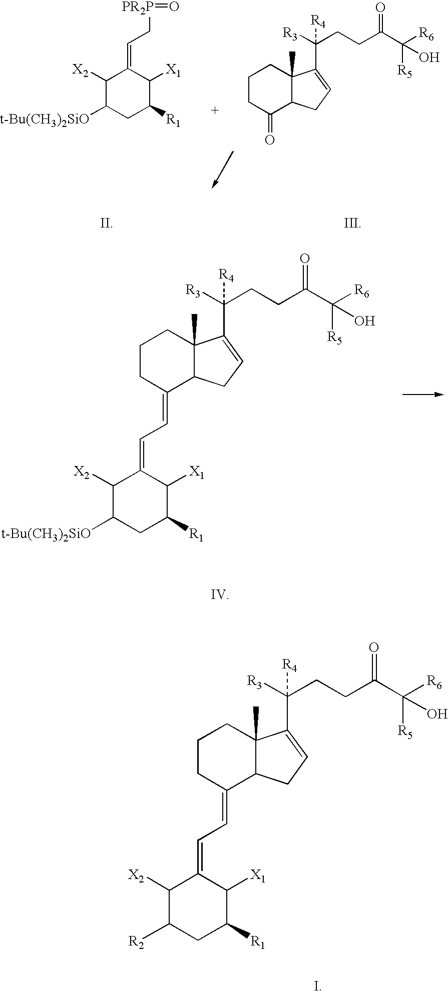
wherein X₁, X₂, R₃, R₄, R₅ and R₆ are as defined above.

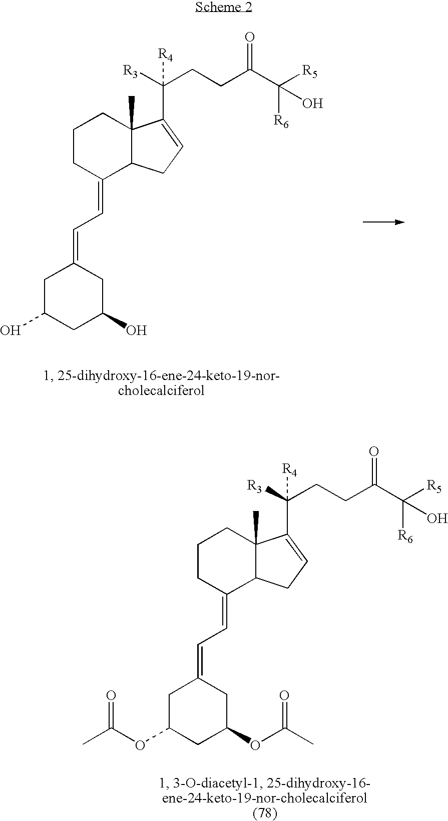

Referring to Schemes 1 and 3, compounds of formula II are known compounds, and are prepared starting from the known epoxy-ketone of formula V. The compound of formula V is converted to the epoxy-olefin of formula VII by a Wittig reaction. Reduction with LiAlH4 to the compound VIII and protection of the hydroxy group resulted in compound IX. Then, the ene reaction of forumula IX with the known hydroxy-conjugated ketone X (R₅=R₆=CH₃) in tetrahydrofuran, in the presence of Lewis acid (CH₃)₂ Al Cl, provides the compound XI featuring the C, D-rings and full side chain of the target vitamin D analogs. Finally, removal of the silyl group and oxidation provides the key intermediate, Ketone of formula III.

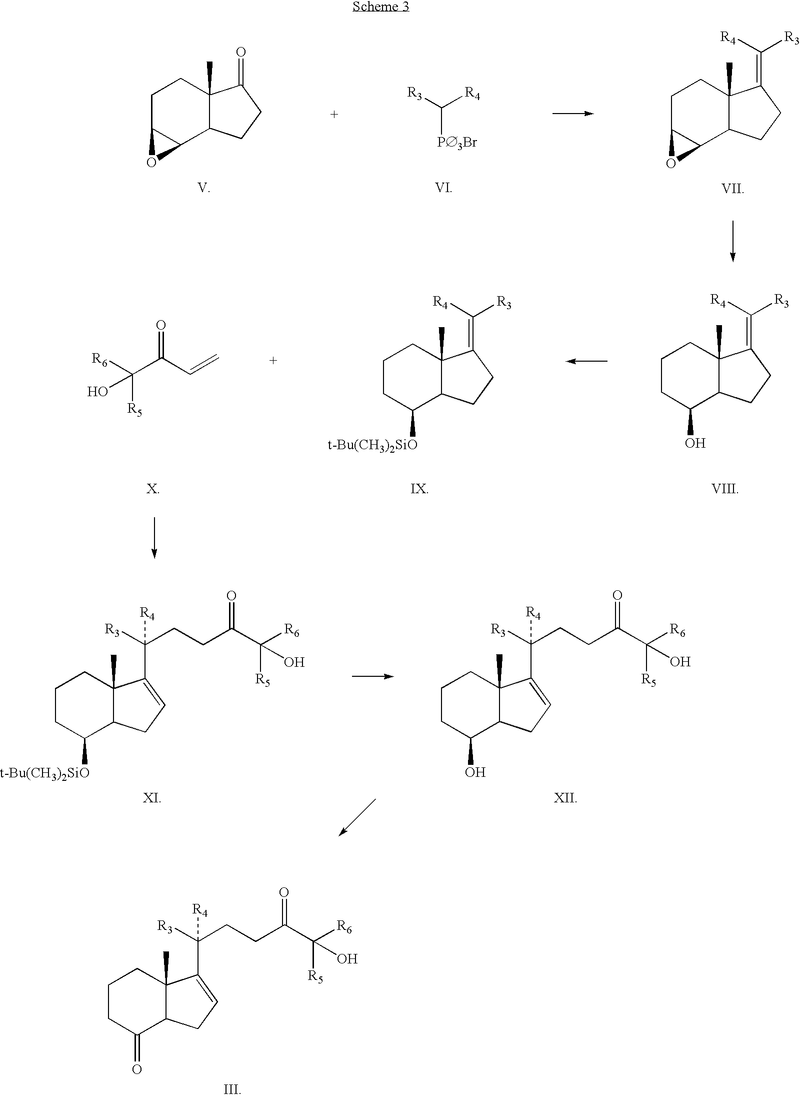

Referring to Scheme 2, 0.032 g of 1,25-dihydroxy-16-ene-24-keto-19-nor-cholecalciferol was dissolved in 0.8 ml pyridine, cooled in bath and treated with 0.2 ml acetic anhydride for 7 hours at room temperature and for 14 hours in a refrigerator. It was then diluted with 1 ml of water, stirred for 10 min in an ice bath, diluted with 5 ml water and 20 ml ethyl acetate. The organic layer was washed with 3×5 ml of water, then with 5 ml saturated sodium bicarbonate, then with brine, dried over sodium sulfate and evaporated. The oily residue was taken up in 1:6 ethyl acetate-hexane, then flash chromatographed on a 13.5×110 mm column using 1:6 ethyl acetate-hexane as mobile phase for fractions 1-5, 1:4 ethyl acetate-hexane for the remaining fractions. Fractions 11-14 were pooled and evaporated to give 0.0184 g of the title compound (2).

IV. Biological Examples

As described in the following examples, the Inventors' finding that calcitriol and Vitamin D₃ analogues can have an effect on the growth and function of bladder cells has been proven in in vitro models by culturing human stromal bladder cells and has been confirmed in a preclinical in vivo validated model.

### Example 47

### The activity of Calcitriol and Vitamin D₃ Analogues on the Growth and Function of Bladder Cells

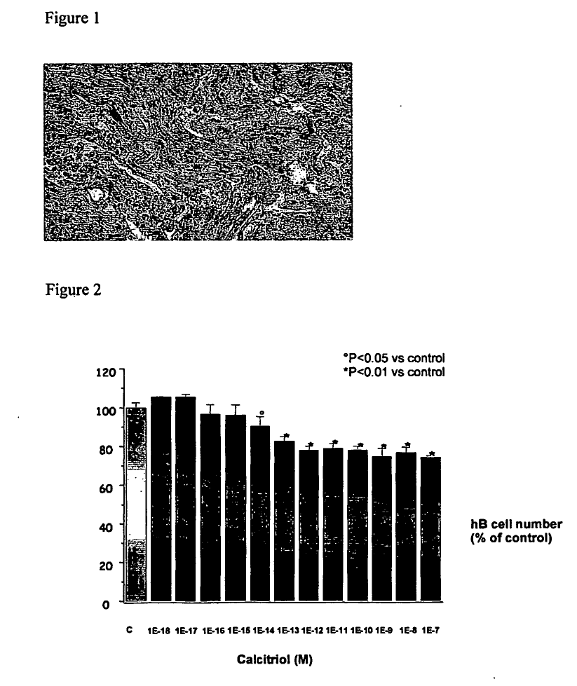

The Inventors' finding that calcitriol and Vitamin D₃ analogues can have an effect on the growth and function of bladder cells has been proven in in vitro models by culturing human stromal bladder cells The Inventors confirmed the presence of vitamin D receptors (VDRs), as previously reported in the literature, on these cells (see below in FIG. 1).

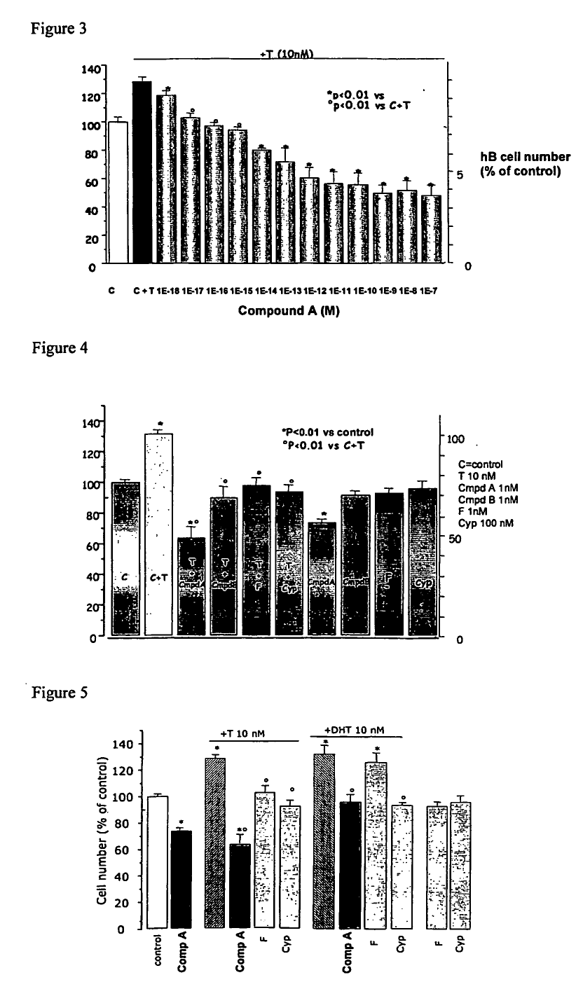

In these models, calcitriol (the activated form of vitamin D₃) and other vitamin D₃ analogues have been shown to be effective in inhibiting the basal (FIG. 2) and testosterone-stimulated (FIG. 3) growth of bladder cells. This activity, never reported before, is dose dependent with an IC₅₀ of 9.8±7×10⁻¹⁵ for calcitriol (1,25-dihydroxycholecalciferol) (on basal cells) and of 1.6±7×10⁻¹⁵ for 1-alpha-fluoro-25-hydroxy-16,23e-diene-26,27-bishomo-20-epi-cholecalciferol (“Compound A”/“Cmpd A” in the Figures) (on stimulated cells) (see FIG. 2 and FIG. 3).

This effect, demonstrated also with other vitamin D₃ analogues (e.g. 1,25-dihydroxy-16-ene-23-yne cholecalciferol described in U.S. Pat. No. 5,145,846 and referred to as “Compound B”/“Cmpd B” in these Examples and the Figures) was, in some cases, significantly greater than that of anti-androgens widely used in the treatment of uro-genital diseases, such as finasteride (FIG. 4).

A similar investigation was performed on a number of other vitamin D compounds and the results (expressed as −Log IC₅₀) are shown in the table below. Data in the table refers to inhibitors effect of the compound on basal human bladder cell growth in cells which are not stimulated with testosterone or (in one case) are stimulated. The maximum tolerated dose (MTD) in rats is also listed for each compound.

| | | MTD | |
|---|---|---|---|
| Compound | −Log IC₅₀ | (ug/kg) | |
| Compound A* | 11.2 ± 0.57 | 100 | 628 |
| 1,25-Dihydroxy-21-(2R,3- | 4.62 ± 2.2 | 30 | 2 |
| dihydroxy-3-methyl-butyl)-20R- | | | |
| cholecalciferol | | | |
| 1,3-Di-O-acetyl-1,25-dihydroxy- | 9.65 ± 0.36 | 1 | 10 |
| 16-ene-cholecalciferol* | | | |
| 1,3-Di-O-acetyl-1,25-dihydroxy- | 6.4 ± 1 | 30 | 35 |
| 20-cyclopropyl-cholecalciferol | | | |
| 1,3-Di-O-acetyl-1,25-dihydroxy- | >2 | 1 | 7 |
| 23-yne-cholecalciferol | | | |
| 1,3-Di-O-acetyl-1,25-dihydroxy- | 10.3 ± 0.26 | 10 | 8 |
| 16-ene-23-yne-cholecalciferol* | | | |
| 1,25-Dihydroxy-16,23Z-diene-20- | 7.1 ± 0.68 | 1 | 56 |
| cyclopropyl-26,27-hexafluoro- | | | |
| cholecalciferol | | | |
| 1,3-Di-O-acetyl-1,25-dihydroxy- | 7.4 ± 0.57 | 0.1 | 29 |
| 16,23Z-diene-26,27-hexafluoro- | | | |
| 19-nor-cholecalciferol | | | |
| 1,25-Dihydroxy-16,23E-diene-20- | 10.8 ± 0.34 | 0.3 | 51 |
| cyclopropyl-26,27-hexafluoro- | | | |
| cholecalciferol* | | | |
| 1,3,25-Tri-O-acetyl-1,25- | 7.4 ± 0.77 | 10 | 27 |
| dihydroxy-20-cyclopropyl-23-yne- | | | |
| 26,27-hexafluoro-19-nor- | | | |
| cholecalciferol | | | |
| 1,3-Di-O-acetyl-1,25-dihydroxy- | 8.92 ± 0.29 | 10 | 28 |
| 20-cyclopropyl-23-yne-26,27- | | | |
| hexafluoro-19-nor-cholecalciferol* | | | |
| 1,25-dihydroxy-21(3-hydroxy-3- | 1.5 ± 4.6 | 0.2 | 72 |
| trifluoromethyl-4-trifluoro- | | | |
| butynyl)-26,27-hexadeutero-19- | | | |
| nor-20S-cholecalciferol | | | |
| 1,3-Di-O-acetyl-1,25-dihydroxy- | 11.38 ± 0.39 | 3 | 9 |
| 16,23E-diene-cholecalciferol* | | | |
| 1,25-dihydroxy-16-ene-20- | 7.77 ± 0.44 | 1 | 62 |
| cyclopropyl-cholecalciferol | | | |
| 1,3-Di-O-acetyl-1,25-dihydroxy- | >2 | 30 | 30 |
| 20-cyclopropyl-23-yne- | | | |
| cholecalciferol | | | |
| 1,3-Di-O-acetyl-1,25-dihydroxy- | 6.21 ± 0.66 | 300 | 31 |
| 16-ene-24-keto-19-nor- | | | |
| cholecalciferol | | | |
| 1,3-Di-O-acetyl-1,25-dihydroxy- | 6.7 ± 0.36 | 10 | 33 |
| 20-cyclopropyl-23Z-ene-26,27- | | | |
| hexafluoro-19-nor-cholecalciferol | | | |
| 1,3-Di-O-acetyl-1,25-dihydroxy- | 8.7 ± 0.27 | 10 | 19 |
| 16-ene-23-yne-26,27-hexafluoro- | | | |
| cholecalciferol* | | | |
| 1,25-Dihydroxy-16-ene-20- | 2.45 ± 2.47 | 0.3 | 48 |
| cyclopropyl-23-yne-26,27- | | | |
| hexafluoro-cholecalciferol | | | |
| 1,3-Di-O-acetyl-1,25-dihydroxy- | 9.2 ± 0.5 | 3 | 24 |
| 16-ene-19-nor-cholecalciferol* | | | |
| 1,25-dihydroxy-21-(3-hydroxy-3- | 5.01 ± 2 | No Data | BLA2 |
| methylbutyl)-19-nor- | | | |
| cholecalciferol | | | |
| 1,25-dihydroxy-21-(3-hydroxy-3- | 13.42 ± 0.85 | No Data | BLA2 |
| methylbutyl)-19-nor- | | | |
| cholecalciferol^{a} | | | |
| 1,3-Di-O-acetyl-1,25-dihydroxy- | 3.73 ± 2.3 | 30 | 25 |
| 16-ene-23-yne-19-nor- | | | |
| cholecalciferol | | | |
| 1,3-Di-O-acetyl-1,25-dihydroxy- | 8.8 ± 0.4 | 0.3 | 32 |
| 20-cyclopropyl-23E-ene-26,27- | | | |
| hexafluoro-19-nor-cholecalciferol* | | | |

Compounds marked in the table with an asterisk (*) are those which are of particular interest in the context of the invention (these having the highest −LogIC₅₀ values for unstimulated cells).

The second entry in the table of 1,25-dihydroxy-21-(3-hydroxy-3-methylbutyl)-19-nor-cholecalciferol marked a indicates data derived from use of stimulated cells (all the other data in the table relates to use of unstimulated cells).

### Example 48

### The Effect of Vitamin D₃ Analogue Compound A on Basal and Stimulated Human Bladder Cell Proliferation and Survival and Apoptosis

In order to further investigate the effects of anti-androgens or Compound A on androgen-stimulated hBC growth, cells were incubated for 48 h with Compound A (1 nM) or anti-androgens (finasteride, F, 1 nM; cyproterone acetate, Cyp, 100 nM) in the presence or absence of testosterone, T (10 nM) or dihydrotestosterone, DHT (10 nM).

Results are expressed as percentage variation (mean±SEM) over their relative controls and derived from at least three different experiments obtained from three distinct hBC cell preparations. *P<0.05 (vs. control); °P<0.01 (vs. androgen-treated cells). Results are shown in FIG. 5. FIG. 5 shows some of the same data as FIG. 4 but also shows that Compound A inhibits hBC proliferation which is stimulated by the androgen DHT, unlike finasteride which had no significant effect.

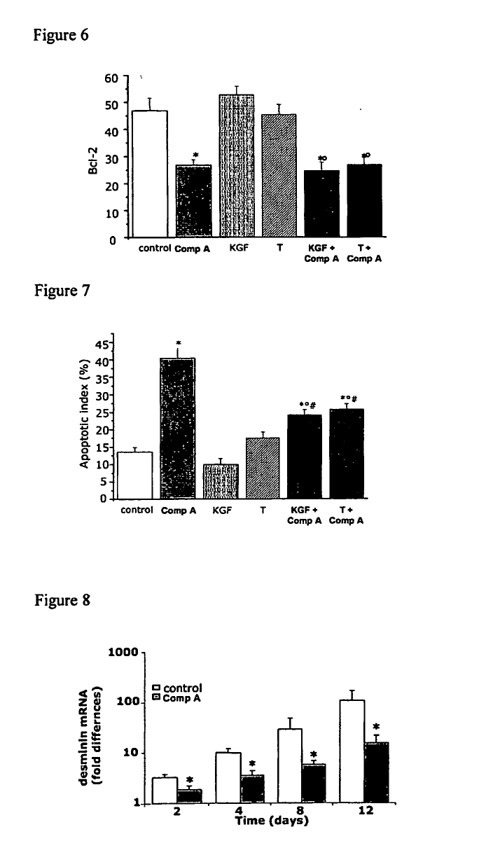

In order to further investigate the effect of Compound A (10 nM), KGF (10 ng/ml) and T (10 nM) on bcl-2 expression in hBC, Bcl-2 protein expression was evaluated by immunocytochemistry as previously described (Crescioli, C. et al. (2000) J. Clin. Endocrinol. Metab. 85:2576-83). After incubation with the indicated stimuli, slides were washed twice with PBS pH 7.4 and fixed in 3.7% paraformaldehyde in PBS for 15 min at room temperature, followed by permeabilization in 3.7% paraformaldehyde in PBS, containing 0.1% Triton X-100 for 15 min at room temperature. Anti-Bcl-2 mAb (1:40) diluted in PBS containing 2% BSA was added to the slides and incubated overnight at 4° C. Slides were washed three times (5 min) in PBS and incubated 45 min at room temperature with 2% BSA-PBS, containing the secondary antibody (dilution 1:1000). After three washes in PBS, the slides were examined with a phase contrast microscope (Nikon mocrophot-FX microscopes; Nikon, Kogaku, Tokyo, Japan). Slides lacking the primary antibody or stained with the corresponding non-immune serum served as controls. The percentage of bcl-2 stained cells was calculated by counting the number of immunopositive cells divided by the total cell number in each of at least five separate fields per slide. Data are derived from three different experiments obtained from three separate hBC preparations. *P<0.05 (vs. control); °P<0.05 (vs. KGF or T-treated cells). Results are shown in FIG. 6. FIG. 6 shows that Compound A significantly inhibits bcl-2 expression alone and also in the presence of KGF or testosterone.

In order to investigate the effect of Compound A (10 nM), KGF (10 ng/ml) and T (10 nM) on DNA fragmentation in hBC, the apoptotic index was obtained from in situ end labelling (ISEL) experiments (see Crescioli et al (2004) Eur J Endocrinol. 150:591-603.) and represents the number of stained nuclei divided by the total cell number in each of at least five separate fields per slide. Results are expressed as mean±SEM) and obtained from three different experiments derived from three distinct hBC preparations. *P<0.05 (vs. control); °P<0.05 (vs. Compound A-treated cells); #P<0.05 (vs. KGF- or T-treated cells) and shown in FIG. 7. FIG. 7 shows that Compound A significantly increases the apoptotic index alone and also in the presence of KGF or testosterone.

Taken together the results shown in FIGS. 6 and 7 demonstrate the significant effect that Compound A has on inducing apoptosis in stimulated and unstimulated hBC.

### Example 49

### Effect of Compound A on Desmin Gene and Protein Expression in hBC

The initial stages of bladder hypertrophy are characterised by a tension-induced up-regulation of contractile and cytoskeleton proteins with a net increase in the desmin/actin ratio (Berggren, T. et al. (1996) Urol. Res. 24:135-40). Desmin is a smooth-muscle specific filament which is associated with smooth muscle alpha-actin but still with unknown function and regulation.

To detect desmin both at gene or protein level hBC cells were seeded in their growth medium onto 10 mm diameter culture dishes or onto sterile glass slides (about 10⁴ cells/ml), for mRNA or immunocytochemical analysis, respectively. hBC cells at about 30% confluency, after overnight starvation in serum-free medium were incubated in phenol red- and serum-free medium containing 0.1% BSA with or without Compound A (10⁻⁸ M) for 2, 4, 8 and 12 days, and the medium was changed every 2 days. Cells were harvested for mRNA or protein analysis by Taqman or Western blot analysis, respectively, and the slides were processed for specific protein immunocytochemical detection Quantitative analysis using real-time RT-PCR of desmin mRNA expression in serum-starved hBC treated with Compound A (10 nM, grey columns) was examined at different time points (2-12 days). Results are derived from five different experiments from three distinct hBC preparations and are expressed as fold increase compared to time zero. *P≦0.01 or °P=0.04 vs. control, open columns and are shown in FIG. 8.

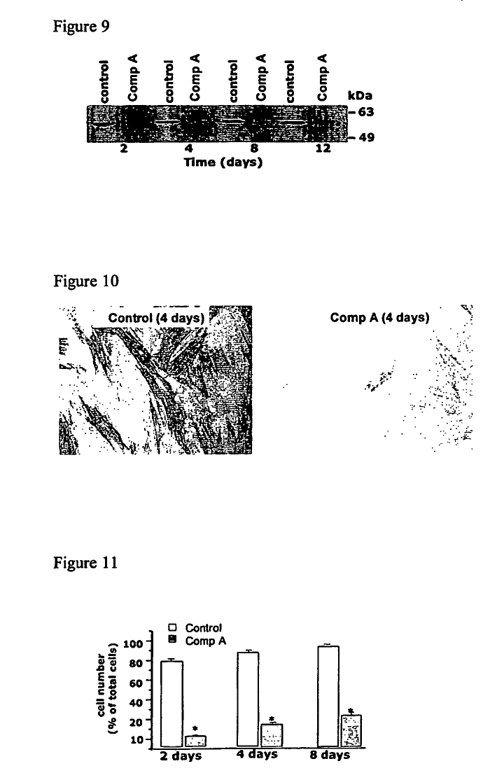

Western blot detection of desmin in hBC was conducted as follows: thirty μg of proteins were separated by 10% SDS-PAGE, transferred onto nitrocellulose membrane, and probed with anti-desmin antibody (1:1000). Results are shown in FIG. 9. A band of about 58 kDa was detected in each sample of hBC. Compound A (10 nM) decreased desmin protein expression at any time point tested. Molecular weight markers (kDa) are indicated at the right of the blot. Results are representative of three independent experiments performed using separate hBC preparations. Immunocytochemical detection of desmin in hBC was conducted as follows: Cells were seeded onto sterile glasses, treated with Compound A (10 nM) and processed at the indicated time points with an anti-desmin antibody (1:1000). Results are shown in FIGS. 10 and 11. The microphotographs reported in FIG. 10 shows results obtained after a 4 day incubation with Compound A (10 nM, right microphotograph, magnification×150) or vehicle (left microphotograph, magnification×150).

Quantification of three separate experiments from three distinct preparations of hBC is shown in FIG. 11 (control, open columns; Compound A, grey columns). The percentage of desmin-positive cells was calculated by counting the number of stained cells divided by the total cell number in each of at least five separate fields per slide. *P<0.01 vs their relative control. In summary: in hBC, prolonged serum starvation induced a progressive increase in smooth muscle specific intermedate filament (desmin) expression which, as shown in FIGS. 8-11, was almost completely counteracted by Compound A. Desmin overexpression in hBC may be expected to cause or exacerbate bladder dysfunction which may therefore be expected to be treated by Compound A.

### Example 50

### Effect of Compound A on Vimentin Gene and Protein Expression in hBC

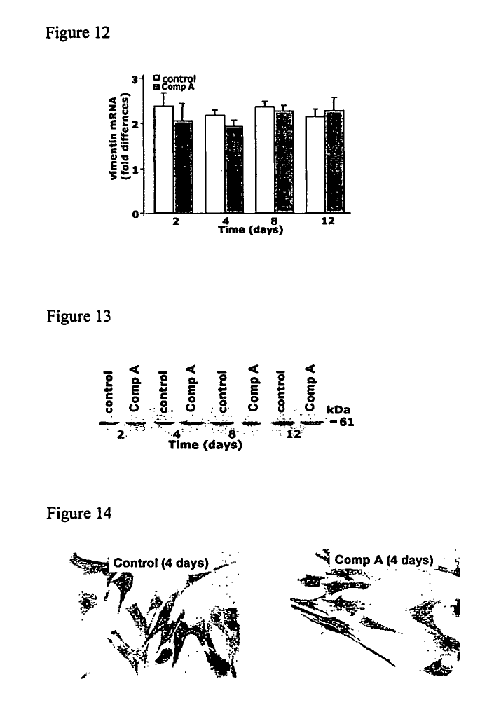

Vimentin was detected (mRNA and protein) as per the method for desmin described in Example 1B. Vimentin is a fibroblastic cell marker. Quantitative analysis using real-time RT-PCR of vimentin mRNA expression in serum-starved hBC treated with Compound A (10 nM) was examined at different time points (2-12 days). Results are shown in FIG. 12. Results are derived from five different experiments from three distinct hBC preparations and are expressed as fold increase compared to time zero. Control, open columns; Compound A, grey columns.

Western blot detection of vimentin in hBC was performed as follows: Thirty ug of proteins were separated by 10% SDS-PAGE, transferred onto nitrocellulose membrane, and probed with anti-vimentin antibody (1:1000). Results are shown in FIG. 13. A band of about 61 kDa was detected in each sample of hBC. Compound A (10 nM) failed to affect vimentin protein expression at any time point tested. Results are representative of three independent experiments performed using separate hBC preparations.

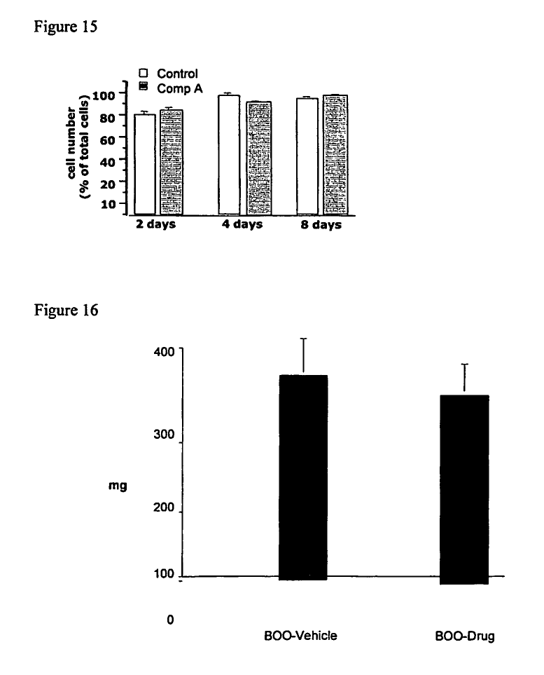

Immunocytochemical detection of vimentin in hBC was conducted as follows: Cells were seeded onto sterile glasses, treated with Compound A (10 nM) and processed at the indicated time points with an anti-vimentin antibody (1:1000). Results are shown in FIGS. 14 and 15. The microphotographs reported in FIG. 14 shows results obtained after a 4 day incubation with Compound A (10 nM, right microphotograph, magnification×150) or vehicle (left microphotograph, magnification ×150). Quantification of three separate experiments from three distinct preparations of hBC is shown in FIG. 15 (control, open columns; Compound A, grey columns). The percentage of vimentin positive cells was calculated by counting the number of stained cells divided by the total cell number in each of at least five separate fields per slide. The failure of Compound A to inhibit the fibroblastic cell marker vimentin provides confirmatory evidence that the effect on desmin described in Example 1B is a specific and useful effect.

### Example 51

### The Effect of Vitamin D₃ Analogues on Bladder Dysfunction in a Bladder Outlet Obstruction Model

Experimental

1. Materials

1.1. Animals:

Female Sprague-Dawley rats, weighing 200-250 g

1.2. Grouping

Group A: BOO rats, treated with the vitamin D analogue over 2 weeks, beginning at day 1 after creation of the obstruction (n=12)

Group B: BOO rats, treated with vehicle over 2 weeks, beginning at day 1 after creation of the obstruction (n=12)

Group C: Sham operated rats, treated with the vitamin D analogue over 2 weeks, beginning at day 1 after surgery (n=12)

1.3. Studies:

a) Cystometry (˜18 hours after last administration of the drug/vehicle, 12 hours after removal of the obstructing ligature) under conscious conditions.

b) Measurements of bladder weight.

c) In vitro investigations.

2. Methods

2.1. BOO:

The bladder and urethrovesical junction were exposed through a lower abdominal midline incision. A 0.9 mm metal rod was placed alongside the proximal urethra and a 3-0 silk ligature was tied tightly around the urethra and the rod, which was consequently removed. Sham surgery was performed accordingly, without placing the ligature. After 13 days the ligature was removed and a catheter was inserted into the bladder dome and tunneled subcutaneously.

2.2. Cystometry:

The following morning after insertion of the catheter, the cystometric investigation was performed without any anesthesia or restraint in a metabolic cage. The amount of voided urine was measured by means of a fluid collector, connected to a force displacement transducer. The bladder was continuously filled with saline at room temperature. The catheter was also connected to a pressure transducer. After a stabilization period of 30-60 minutes, when reproducible voiding patterns are achieved, the following parameters were recorded over a period of 30 min: Basal bladder pressure, micturition pressure, threshold pressure, micturition interval and volume, and non-voiding contractions. The amount of residual urine was investigated manually 3 times, at the end of the cystometry. Bladder capacity was calculated based on the measured values.

2.3. In Vitro Investigations

2.3.1. Preparations:

After completion of the cystometries, the rats were sacrified by carbon monoxide asphyxiation followed by exsanguination. The abdomen was accessed through a lower midline incision whereafter the symphysis was opened. The bladder was carefully dissected free, and immediately placed in chilled Krebs solution, and strip preparations were dissected.

2.3.2 Recording of Mechanical Activity:

The bladder and urethra were separated at the level of the bladder neck, and semicircular strips were prepared from the middle third of the detrusor (1×2×5 mm). All preparations were used immediately after removal.

The strips were transferred to 5 ml tissue baths containing Krebs solution. The Krebs solution was maintained at 37° C. and bubbled continuously with a mixture of 95% O₂ and 5% CO₂, resulting in a pH of 7.4. The strips were suspended between two L-shaped hooks by means of silk ligatures. One hook was connected to a movable unit allowing adjustment of passive tension, and the other to a Grass FT03C (Grass Instruments Co, MA, USA) force transducer. Isometric tension was recorded using a Grass polygraph (7D). After mounting, the strips were stretched to a passive tension of 4 mN (the same tension for all preparations) and allowed to equilibrate for 45-60 min before further experiments were performed.

2.3.3. Electrical Field Stimulation

Electrical field stimulation (EFS) was accomplished by means of two platinum electrodes placed on either side of the preparations, and was performed using a Grass S48 or S88 stimulator, delivering single square wave pulses at selected frequencies. The train duration was 5 s, the pulse duration 0.8 ms, and the stimulation interval 2 min. The polarity of the electrodes was shifted after each pulse by means of a polarity changing unit.

2.3.4 Procedure:

Each experiment was started by exposing the preparations to a high K⁺ (124 mM) Krebs solution until two reproducible contractions are obtained. Then the following experiments were carried out:

a) Electrical stimulation of nerves was performed and frequency-response relations obtained, in the presence and absence of atropine.

b) Concentration-response curves were constructed for carbachol and ATP

Results

The validated bladder outlet obstruction rat model described above was used to test the ability of vitamin D₃ analogues to control and treat bladder dysfunction. The objective was to evaluate whether a vitamin D₃ analogue (1-alpha-fluoro-25-hydroxy-16,23e-diene-26,27-bishomo-20-epi-cholecalciferol-Compound “A”) at the dose of 150 ug/kg/daily can prevent bladder hypertrophy and bladder dysfunction such as bladder overactivity.

In this model a ligature was surgically placed around the outlet of the catheterized bladder, so that when the catheter was removed, the bladder experienced increased urethral resistance. The rats underwent continuous cystometry to evaluate bladder function. In addition the contractile properties of isolated bladder preparation in response to nerve stimulation and exogenous stimuli in vitro were investigated under electrical field stimulation (EFS).

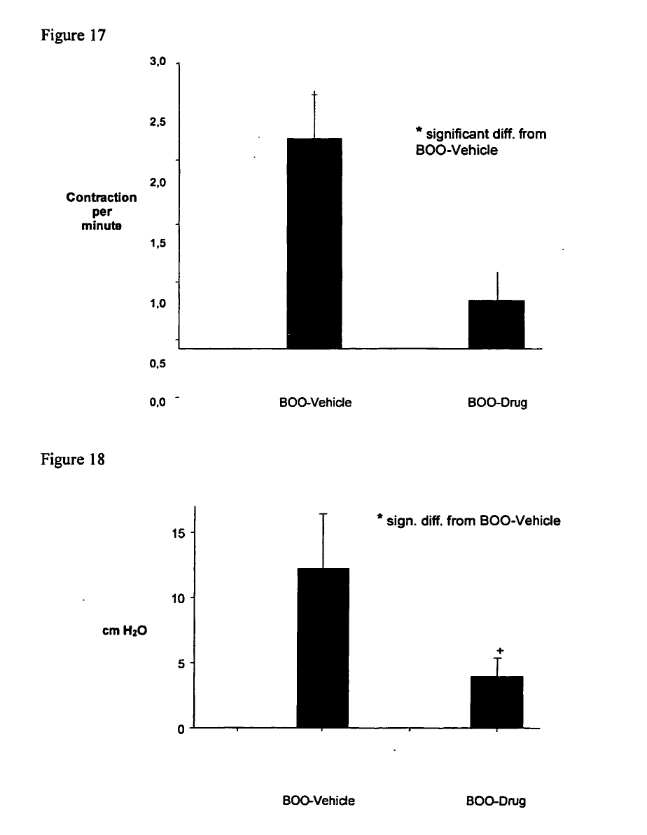

The following cystometric parameters were investigated (see FIGS. 16-20):

micturition pressure (the maximum bladder pressure during micturition)

bladder capacity (residual volume after voiding plus the volume of saline infused to induce the void)

micturition volume (volume of the expelled urine)

residual urine (bladder capacity minus micturition volume)

and

frequency and amplitude of spontaneously occurring changes intravesical pressure (non-voiding contractions).

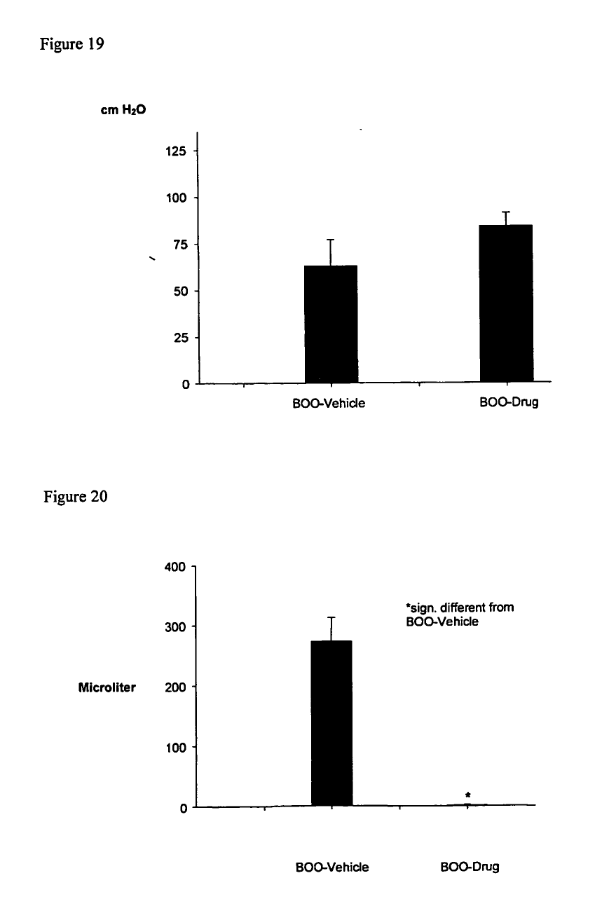

In this model the vitamin D₃ analogue under evaluation had a beneficial effect on bladder function. This effect was evident in the normal bladder and is maintained in bladder outlet obstruction. In particular significant differences versus vehicle were observed in:
spontaneous non-voiding contraction frequency and amplitude (FIGS. 15 and 16);
residual urine (absent with the active compound, FIG. 20);
micturition pressure (FIG. 19).

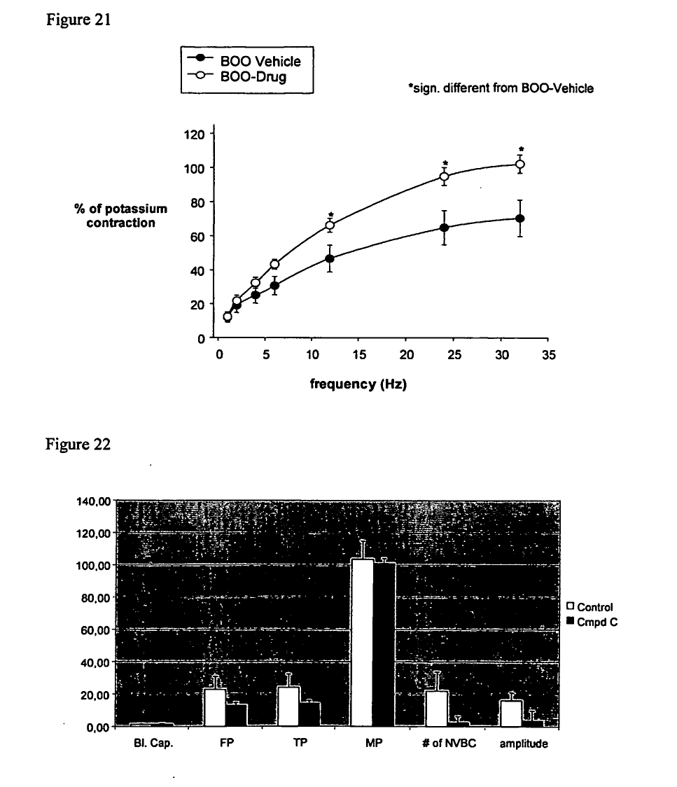

In addition a beneficial effect on bladder function has been confirmed in the in vitro tests:
K response;
response to EFS (FIG. 21);
response to carbachol.

Finally a slight decrease in bladder weight was observed with the vitamin D₃ analogue tested (FIG. 16).

These data demonstrate the use of vitamin D analogues (in the dose range from 50 ug to 300 ug-equivalent to approximately 0.725 to 5 ug/kg of body mass in humans) in the prevention and treatment of bladder dysfunction, such as overactive bladder.

### Example 52

### Soft Gelatin Capsule Formulation I

| Item | Ingredients | mg/Capsule |
|---|---|---|
| 1 | 1-alpha-fluoro-25-hydroxy-16,23E- | 10.001-0.02 |
| | diene-26,27-bishomo-20-epi-cholecalciferol | |
| 2 | Butylated Hydroxytoluene (BHT) | 0.016 |
| 3 | Butylated Hydroxyanisole (BHA) | 0.016 |
| 4 | Miglyol 812 qs. | 160.0 |

Manufacturing Procedure:
1. BHT and BHA is suspended in Miglyol 812 and warmed to about 50° C. with stirring, until dissolved.
2. 1-alpha-fluoro-25-hydroxy-16,23E-diene-26,27-bishomo-20-epi-cholecalciferol is dissolved in the solution from step 1 at 50° C.
3. The solution from Step 2 is cooled at room temperature.
4. The solution from Step 3 is filled into soft gelatin capsules.
Note:
All manufacturing steps are performed under a nitrogen atmosphere and protected from light.

### Example 53

### Oral Dosage Form Soft Gelatin Capsule

A capsule for oral administration is formulated under nitrogen in amber light: 150 ug of Compound A in 150 mg of fractionated coconut oil (Miglyol 812), with 0.015 mg butylated hydroxytoluene (BHT) and 0.015 mg butylated hydroxyanisole (BHA), filled in a soft gelatin capsule.

### Example 54

### Oral Dosage Form Soft Gelatin Capsule

A capsule for oral administration is formulated under nitrogen in amber light: 75 ug of Compound A in 150 mg of fractionated coconut oil (Miglyol 812), with 0.015 mg butylated hydroxytoluene (BHT) and 0.015 mg butylated hydroxyanisole (BHA), filled in a soft gelatin capsule.

### Example 55

### Soft Gelatin Capsule Formulation II

| Item | Ingredients | mg/Capsule |
|---|---|---|
| 1 | 1-alpha-fluoro-25-hydroxy-16,23E- | 10.001-0.02 |
| | diene-26,27-bishomo-20-epi-cholecalciferol | |
| 2 | di-.alpha.-Tocopherol | 0.016 |
| 3 | Miglyol 812 qs. | 160.0 |

Manufacturing Procedure:
1. Di-alpha-Tocopherol is suspended in Miglyol 812 and warmed to about 50° C. with stirring, until dissolved.
2. 1-alpha-fluoro-25-hydroxy-16,23E-diene-26,27-bishomo-20-epi-cholecalciferol is dissolved in the solution from step 1 at 50° C.
3. The solution from Step 2 is cooled at room temperature.
4. The solution from Step 3 is filled into soft gelatin capsules.

Example 5: Evaluation of the effect of Vitamin D₃ analogues on bladder function in an in vivo model-cyclophosphamide (CYP) induced chronic IC in rats

The rat model of chemical cystitis induced by intraperitoneal injection of CYP has been well accepted. CYP is used in clinical practice in the treatment of a number of malignant tumors. One of its metabolites, acrolein, is excreted in urine in large concentrations causing hemorrhagic cystitis associated with symptoms of urinary frequency, urgency and pelvic pain. The inflammatory process is characterized by changes in gross histology of bladder, increase in number and distribution of inflammatory cell infiltrates (mast cells, macrophage, PMNs), cyclo-oxygenase-2 expression and prostaglandin production, growth factor and cytokine production. The rat model of chemical cystitis closely resembles interstitial cystitis, a chronic, painful urinary bladder syndrome and has been used for the testing of therapeutic agents in the past.

This model was used to test the effects of 1,25-dihydroxyvitamin D₃ analogue in rats with CYP-induced cystitis. The effects of the treatment on the cystometric parameters in a conscious freely moving rat with CYP-induced cystitis were monitored. The following cystometric parameters were recorded in each animal:

bladder capacity

filling pressure (pressure at the beginning of the bladder filling)

threshold pressure (bladder pressure immediately prior to micturition)

micturition pressure (the maximal bladder pressure during micturition)

presence or absence of non-voiding bladder contractions (increases in bladder pressure of at least 10 cm H₂O without release of urine) amplitude of non-voiding bladder contraction.

Animals: Wistar rats weighing 125-175 g were used. Two groups of animals had a tube implanted into the urinary bladder for intravesical pressure recording. Following recovery all animals received three intraperitoneal injections of CYP and subsequently were divided into the treatment and sham control groups.

Treatment group: Rats treated with oral 1,25-dihydroxyvitamin D₃ analogue 1,3-di-O-acetyl-1,25-dihydroxy-16,23Z-diene-26,27-hexafluoro-19-nor-cholecalciferol (“Compound C”) for 14 days (daily dose of 0.1 μg/kg)

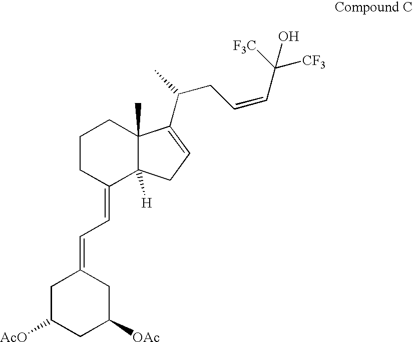

Control group: Rats treated with oral vehiculum (miglyol) in the dose identical to that delivered in the treatment group

Cystometry was performed 24 hours following the last dose of the drug or vehiculum on awake freely moving animals.

Number of animals per group:

| | |
|---|---|
| Sham control animals | 4 |
| Treated animals | 3 |

Methods
Implantation of the Polyethylene Tubing into the Urinary Bladder:

A lower midline abdominal incision was performed under general inhalation anesthesia (isoflurine with O₂) and polyethylene tubing (PE-50, Clay Adams, Parsippany, N.J.) with the end flared by heat was inserted into the dome of the bladder and secured in place with a 6-0 prolene purse string suture. The distal end of the tubing was heat-sealed, tunneled subcutaneously and externalized at the back of the neck, out of the animal's reach. Abdominal and neck incisions were closed with 4-0 nylon sutures.

Intraperitoneal Injection of Cyclophosphamide:

Following recovery (5 days) subject animals underwent three intraperitoneal injections of CYP (Sigma Chemical, St. Louis, Mo.; 75 mg/kg each, intraperitoneal) over the period of nine days. On the tenth day following the first CYP injection the sham control animals received the vehicle only, whereas the experimental group were treated with the 1,25-dihydroxyvitamin D₃ analogue 1,3-di-O-acetyl-1,25-dihydroxy-16,23Z-diene-26,27-hexafluoro-19-nor-cholecalciferol “Compound C” (delivered using gavage). Two weeks following the initiation of the treatment animals underwent a conscious cystometrogram to assess the function of the urinary bladder.

Cystometrogram:

An animal was placed unrestrained in a cage and the catheter was connected via a T-tube to a pressure transducer (Grass® Model PT300, West Warwick, R.I.) and microinjection pump (Harvard Apparatus 22, South Natick, Mass.). A 0.9% saline solution was infused at room temperature into the bladder at a rate of 10 ml/h. Intravesical pressure was recorded continuously using a Neurodata Acquisition System (Grass® Model 15, Astro-Med, Inc, West Warwick, R.I.). At least three reproducible micturition cycles were recorded after the initial stabilization period of 25-30 min.

Timeline of an Experiment:

| Procedure | Days |
|---|---|
| Acclimation period | 1-5 |
| Tube implantation + recovery period | 6-10 |
| CYP treatment (three doses of 75 mg/kg i.p. every three days) | 11-17 |
| Treatment (sham or active) | 18-31 |
| Cystometric evaluation | 32 |

Results

The data analysis is summarized in Tables 1 and 2 and FIG. 22 in which:

Bl. Cap=bladder capacity (ml)

FP=filling pressure (cmH₂O)

TP=threshold pressure (cmH₂O)

MP=micturition pressure (cmH₂O)

# of NVBC=number of non-voiding bladder contractions

amplitude of NVBC=amplitude of non-voiding bladder contraction

TABLE 1
cystometric parameters for the control group.

| | | | | | # of | Amplitude of |
|---|---|---|---|---|---|---|
| Rat | Bl. Cap. | FP | TP | MP | NVBC | NVBC |
| RB 8 | 1.2 | 15 | 15 | 100 | 22 | 15 |
| | 1.2 | 13 | 18 | 100 | 14 | 14 |
| | 1.1 | 16 | 15 | 82 | 12 | 11 |
| RB10 | 0.7 | 30 | 40 | 110 | 26 | 25 |
| | 0.9 | 32 | 26 | 94 | 32 | 28 |
| | 0.6 | 26 | 26 | 108 | 35 | 16 |
| RB12 | 1.7 | 35 | 40 | 115 | 40 | 17 |
| | 1.7 | 25 | 30 | 125 | 35 | 14 |
| | 1.9 | 30 | 25 | 118 | 22 | 17 |
| RB14 | 1.3 | 16 | 16 | 104 | 10 | 10 |
| | 1.2 | 17 | 17 | 95 | 4 | 8 |
| | 1.1 | 19 | 21 | 92 | 9 | 18 |

TABLE 2
cystometric parameters for the treatment group

| | | | | | # of | amplitude of |
|---|---|---|---|---|---|---|
| Rat | Bl. Cap. | FP | TP | MP | NVBC | NVBC |
| RB7 | 0.7 | 13 | 14 | 98 | 0 | 0 |
| | 0.7 | 14 | 14 | 97 | 0 | 0 |
| | 0.8 | 13 | 14 | 101 | 0 | 0 |
| RB13 | 1.4 | 14 | 15 | 104 | 8 | 11 |
| | 1.9 | 15 | 16 | 105 | 4 | 10 |
| | 1.3 | 14 | 17 | 97 | 8 | 11 |
| RB15 | 2.5 | 12 | 14 | 90 | 0 | 0 |
| | 1.3 | 11 | 12 | 100 | 0 | 0 |
| | 1.5 | 10 | 11 | 108 | 0 | 0 |

Changes were noted in a number of cystometric parameters. Dramatic reductions in both the number and amplitude of non-voiding bladder contractions were observed in the drug treated animals. Less pronounced but still statistically significant reductions in the filling and threshold pressures were also recorded. The treatment did not result in a change of the bladder capacity.

Bladder overactivity associated with chronic cystitis manifests itself in frequent contractions of the bladder wall associated with irritative often painful urinary symptoms. The fact that non-voiding bladder contractions were reduced both in their frequency and amplitude strongly suggest that if the effects on the bladder function in patients with interstitial cystitis will be similar, treatment (e.g., oral treatment) with vitamin D₃ analogues has a potential to relieve these debilitating symptoms. Reduction in filling and threshold pressures is significant from a clinical standpoint because the increased intravesical pressure associated with interstitial cystitis is a condition potentially jeopardizing the upper urinary tract.

This example provides a further demonstration that a vitamin D₃ analogue, 1,3-di-O-acetyl-1,25-dihydroxy-16,23Z-diene-26,27-hexafluoro-19-nor-cholecalciferol (Compound C), has the ability to treat bladder dysfunction.

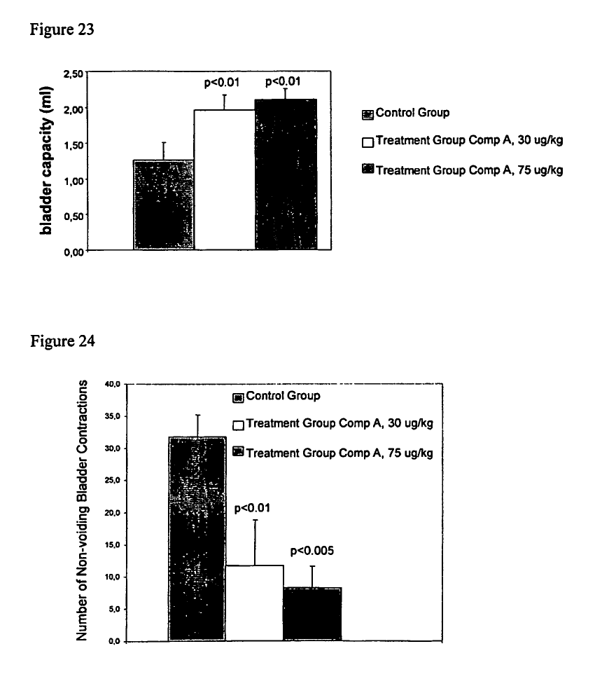

Similar experiments were performed using Compound A as the test compound (30 and 75 ug/kg). The results are shown in FIGS. 23 and 24. These figures show that Compound A also has the ability to treat bladder dysfunction as shown by the increase in bladder capacity and the decrease in non-voiding bladder contractions in this model.

All references including patent and patent applications referred to in this application are incorporated herein by reference to the fullest extent possible. Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

Abbreviations

T testosterone

DHT dihydrotestosterone

GF growth factor

BPH benign prostatic hyperplasia

BOO Bladder Outlet Obstruction

AR Androgen receptors

PSA Prostate Specific Antigen

VDR Vitamin D receptor

hBC human bladder cells

KGF keratinocyte growth factor

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

1. (canceled)

2. A method of prevention or treatment of bladder dysfunction in a patient by administering to a patient in need thereof an effective amount of a Vitamin D compound thereby to prevent or treat bladder dysfunction in said patient.

3. A method according claim 2 which further comprises the step of obtaining or synthesising the Vitamin D compound.

4. A method according to claim 3 wherein the Vitamin D compound is formulated in a pharmaceutical composition together with a pharmaceutically acceptable diluent or carrier.

5. (canceled)

6. (canceled)

7. A kit containing a Vitamin D compound together with instructions directing administration of the Vitamin D compound to a patient in need of prevention or treatment of bladder dysfunction thereby to prevent or treat bladder dysfunction in said patient.

8. A kit according to claim 7 wherein the Vitamin D compound is formulated in a pharmaceutical composition together with a pharmaceutically acceptable diluent or carrier.

9. The method according to claim 2, wherein said Vitamin D compound is a Vitamin D receptor agonist.

10. The method according to claim 9 wherein said Vitamin D receptor agonist is Vitamin D₃ or an analogue thereof.

11. The method according to claim 2, wherein said bladder dysfunction is characterized by the presence of bladder hypertrophy.

12. The method according to claim 2, wherein said bladder dysfunction is overactive bladder.

13. The method according to claim 2 wherein said patient is a male.

14. The method according to claim 13 wherein said male is concurrently suffering from BPH.

15. The method according to claim 2 wherein said patient is a female.

16. The method according to claim 2, wherein the patient is a human.

17. The method according to claim 2, wherein said vitamin D compound is a compound of the formula 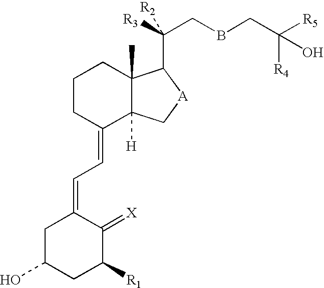
wherein
X is H₂ or CH₂
R₁ is hydrogen, hydroxy or fluorine
R₂ is hydrogen or methyl
R₃ is hydrogen or methyl. When R₂ or R₃ is methyl, R₃ or R₂ must be hydrogen.
R₄ is methyl, ethyl or trifluoromethyl
R₅ is methyl, ethyl or trifluoromethyl
A is a single or double bond
B is a single, E-double, Z-double or triple bond

18. The method according to claim 17, wherein each of R₄ and R₅ is methyl or ethyl.

19. The method according to claim 18 wherein said Vitamin D compound is 1-alpha-fluoro-25-hydroxy-16,23E-diene-26,27-bishomo-20-epi-cholecalciferol, having the formula:

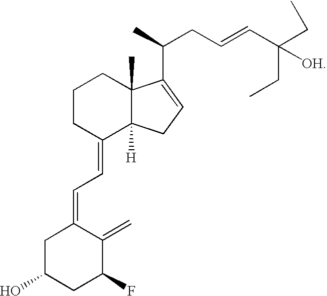

20. The method according to claim 2 wherein said Vitamin D compound is 1,25-dihydroxy-16-ene-23-yne cholecalciferol.

21. The method according to claim 2, wherein said vitamin D compound is 1,3-di-O-acetyl-1,25-dihydroxy-16,23Z-diene-26,27-hexafluoro-19-nor-cholecalciferol, having the formula:

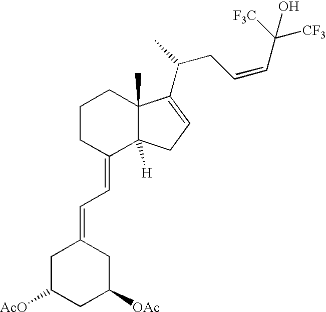

22. The method according to claim 2, wherein said vitamin D compound is calcitriol.
